(19) United States
(12) Patent Application Publication
(10) Pub. No.: US 2014/0045779 A1
(43) Pub. Date: Feb. 13, 2014

(54) COMPOUNDS CONTAINING AN ALICYCLIE STRUCTURE AND ANTI-TUMOR APPLICATION

(71) Applicant: Lifeng Xu, San Diego, CA (US)

(72) Inventor: Lifeng Xu, San Diego, CA (US)

(21) Appl. No.: 14/111,177

(22) PCT Filed: Aug. 29, 2011
(86) PCT No.: PCT/CN2011/079053

§ 371 Date: Oct. 11, 2013

(30) Foreign Application Priority Data
Apr. 26, 2011 (CN) 201110104357.0

(51) Int. Cl. A61K 31/7034; A61K 31/519; C07D 209/48; A61K 31/4035; C07D 491/18; A61K 31/407; C07D 453/02; A61K 31/439; C07D 233/61; A61K 31/4164; C07C 233/08; A61K 31/16; C07H 15/24; A61K 31/69; C07F 5/02; C07D 487/18; A61K 31/53; A61K 45/06; C07D 487/04

(52) U.S. Cl. CPC A61K 31/7034; C07D 487/04; A61K 31/519; C07D 209/48; A61K 31/4035; C07D 491/18; A61K 31/407; C07D 453/02; A61K 31/439; C07D 233/61; A61K 31/4164; C07C 233/08; A61K 31/16; C07H 15/24; A61K 31/69; C07F 5/027; C07D 487/18; A61K 31/53; A61K 45/06
USPC 514/35; 544/281; 514/259.31; 548/474; 514/417; 548/430; 514/411; 546/133; 514/305; 548/338.1; 514/399; 564/196; 514/626; 536/17.9; 514/64; 568/1; 544/219; 514/246

(57) ABSTRACT

This invention relates with anti-tumor activities of new compounds containing an adamantyl group or analogs thereof. The invention also relates with the medication applications of anti-tumor and other diseases by this kind of compounds with the combination of S, P, T structures containing adamantyl group and the formation of stereoisomer, tautomers, prodrug, pharmaceutically acceptable salts, complex salts or solvates to their anticancer application and anticancer agents, which have the following general formula:

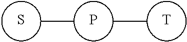

### FIELD OF THE INVENTION

This invention relates with anti-tumor activities of new compounds containing an adamantyl group or analogs thereof. The invention also relates with the medication applications of anti-tumor and other diseases by this kind of compounds.

The literatures showed that only two anticancer patents of compounds containing adamantyl group: U.S. Pat. No. 7,365,231B2, memantine with anti-proliferation activity of glial cells of glial cell proliferation of the brain, neck and glioma cancer; US 2006/0079463, hexamethylene-tetramine with anti-proliferation activity. The rest of literatures are anti-viral applications, U.S. Pat. No. 4,230,725 and U.S. Pat. No. 5,576,355; treatment of Parkinson's disease US 2004/0242493A1, U.S. Pat. No. 4,122,193, U.S. Pat. No. 4,122,193, U.S. Pat. No. 4,064,285; treatment of diabetes, obesity, US2008/0103183A1, US 2008/0312214A1, US 2008/0103183A1, US 2008/0096869A1, U.S. Pat. No. 7,435,833; treatment of multiple sclerosis US 2009/0081259A1; treatment of neurodegenerative diseases, US 2006/0270742A1, U.S. Pat. No. 6,444,702 B1, U.S. Pat. No. 7,326,730 B2, US2008/0009546A1; treatment of measles U.S. Pat. No. 4,386,105; treatment of cerebral ischemia U.S. Pat. No. 5,061,703; treatment of trypanosomiasis, U.S. Pat. No. 6,602,862; treatment of senile dementia, US 20050222271A1; the treatment of nervous system diseases, US 2004/0242493A1, U.S. Pat. No. 4,122,193, U.S. Pat. No. 4,122,193, U.S. Pat. No. 4,064,285; treatment of Alzheimer's disease US2005/0222271 and U.S. Pat. No. 6,384,083, as well as anti-inflammatory, US 20080153850.

Based on previous research Adapalene, Memantine, Tromantadine with anti-tumor activity in our patent application 200910146141.3, 201010561132.3 and 201010561122.X. We found that compounds with adamantyl ring group had anti-tumor activity.

### SUMMARY OF THE INVENTION

The purpose of the present invention is to provide an chain compounds by the combination of S, P, T structures containing adamantyl group and the formation of a stereoisomer, tautomer, prod-rug, pharmaceutically acceptable salt or a complex salt and solvate to their anticancer application and anticancer agents, to pharmaceutical compositions containing these compounds, which have the following general formula

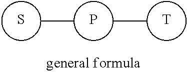

or stereoisomers, tautomers, prodrugs, pharmaceutically acceptable salts, complex salts or solvates thereof, wherein:

S, P, T can be combined independently either in three or in two conponents to form SPT, STP, TSP, PT or ST structures, which is independently an optionally substituted with the carbon-carbon bond or carbon-hetero bond to form ethers, esters, amides, alcohols, aldehydes, ketones, amines, acetal, ketal, oxime and/or hydrazonyl; where S is independently an optionally substituted cyclic group; P is an optionally substituted independently between S and T; T is independently an optionally substituted alkyl and/or adamantyl groups;

S is independently and optionally substituted or fused, saturated or unsaturated, monocyclic, bicyclic, tricycli, teteracyclic, polycyclic, bridged cyclic group, a macrocyclic, midcyclic and/or small cyclic group to form C₃₋₃₀ arylcyclic, aliphatic cyclic, aliphatic heterocyclic group and/or aryl heterocyclic group as the structural formula I, II, III, IV, wherein:

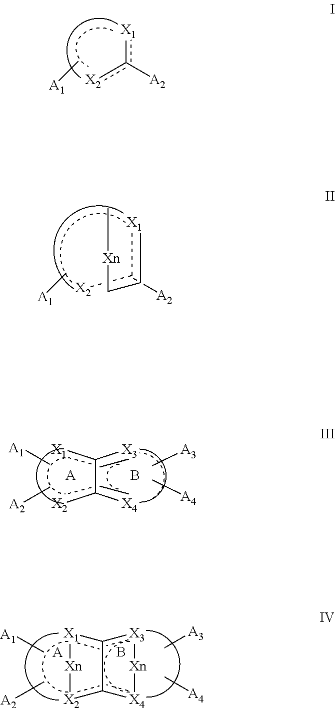

the ring A is optionally substituted independently C₃₋₁₈ alicyclic, arylcyclic, heterocyclic, aliphatic and/or heteroarylcyclic group; ring B is optionally substituted independently C₃₋₁₈ alicyclic, arylcyclic, heterocyclic, aliphatic and/or heteroarylcyclic group; licyclic, arylcyclic, and the aliphatic heterocyclic group or heteroarylcyclic group; A ring was fused with B ring directly or fused to form a bridged ring; P is an optionally substituted independently C₀₋₁₂ alkyl, C₀₋₁₈ aliphatic, C₀₋₁₈ cyclic, arylcyclic, aliphatic heterocyclic, aryl heterocyclic group between S and T to form an independent optionally substituted ether, ester, amide, alcohol, aldehyde, ketone, amine, acetal, ketal, oxime and/or hydrazone group by a formation of the carbon-carbon bond or a carbon-hetero bond with a certain interval of C₀₋₁₂ alkyl, C₀₋₁₈ linear or cyclic aliphatic, arylcyclic, aliphatic and arylheterocyclic, or a heterocyclic group between S and T;

T is an optionally substituted independently adamantyl or adamantyl analog group which contains a C₃₋₃₀ monocyclic, bicyclic, polycycloalkyl, bridged cyclic, cage cyclic, fused cyclic or diamond group containing oxygen, sulfur, nitrogen, phosphine to form optionally substituted mono-adamantane, bi-adamantane, tri-adamantane, polyadamantane or adamantane caged analog with formula V, VI, VII, VIII, IX, X;

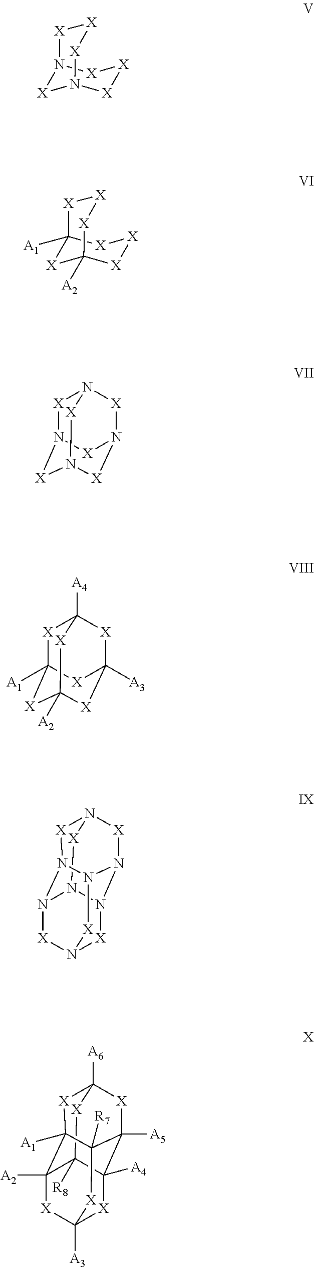

According the formula I, II, III, IV, the dotted lines are optionally substituted single bonds, optionally substituted double bond or a optionally substituted heterocyclic group containing carbon, oxygen, sulfur or nitrogen element;

X₁, X₂, X₃, X₄ are, independently at each occurrence, C═O, C═S, C═NH, C═Rb—Ra, CHOH, CHORb, CHRb or substituent, where Rb contains, independently at each occurrence, one or combination of C, N or P element; Ra is H, H₂, optionally substituted straight-alkyl, optionally substituted branched-alkyl, C₁₋₁₀ optionally substituted saturated alkyl, optionally substituted 1-4 double bond, optionally substituted 1-4 triple bond, optionally substituted unsaturated alkyl, optionally substituted saturated or unsaturated alicyclic, optionally substituted arylcyclic, optionally substituted aryl or optionally substituted heterocyclic, optionally substituted arylheterocyclic, fused heterocyclic group where contains hydroxyl, halogen, oxygen, nitrogen, sulfur, phosphorus element or selenium element;

A₁, A₂, A₃, A₄, A₅, A₆, A₇ or A₈ is, independently at each occurrence, optionally substituted independently cyclic, alkyl, aryl, alicyclic, heterocyclic, aliphatic, aromatic heterocyclic, heterocyclic, glycosyl, multi-hydroxyl, amino acid, phosphate, acyloxyl, phosphoric, sulfonyloxyl, alkoxyl, aryloxyl, heterocyclic oxyl, aryl cyclic, aliphatic heterocyclic oxyl or aryl heterocyclic oxyl group, which containing hydrogen, halougen, oxygen, sulfur, nitrogen or phosphorus element, hydrogen bond, carbon-carbon bond, carbon-hetero bond, and one or a combination;

wherein said sugar or a glycoside bond with carbon-carbon and carbon-hetero atom linkage; substituted oxygen-containing group, oxygen, sulfur, nitrogen or phosphorus, and substituents; including optionally substituted 1-8 separate and independent sugar group or an optionally substituted glycosyl, the sugar group is independently an optionally substituted three-carbon sugar, tetroses, pentoses, hexoses, heptoses, monosaccharides, disaccharides, trisaccharides and polysaccharides or a group; the substituted group is independently an optionally substituted acyloxy, 1-4 phosphono group, alkoxyl, aryloxyl or a heterocyclic; said substituent containing oxygen, sulfur, nitrogen or phosphorus atom, independently an optionally substituted unsaturated or saturated C₁₋₁₀ alkyl, 1-4 double bond or triple bond of the unsaturated aliphatic hydrocarbon group, saturated or unsaturated C₃₋₁₀ alkyl, aryl, alicyclic, heterocyclic, aryl heterocyclic, polycyclic group and or one of combination; glycosyl, multihydroxyl, amino acid, phosphate, acyloxyl, phosphoric, sulfonyloxyl, alkoxyl, aryloxyl, heterocyclic oxyl, aryl cyclic, aliphatic heterocyclic oxyl or aryl heterocyclic oxyl group, non-alicyclic group, an aryl group or a heterocyclic group and, and the introduction of oxygen, sulfur, nitrogen or phosphorus atom independently 3-10 the carbon chain optionally substituted hydrocarbon group, an aromatic ring, polycyclic, aliphatic heterocyclic ring, fused aromatic heterocyclic ring or a heterocyclic cycle and one or a combination.

The compound is selected from the exemplified examples or stereoisomers, tautomers, pharmaceutically acceptable salts, inorganic acid salt, organic acid salt, organic basic salt, organic basic salt, complex salt, prodrugs or solvates thereof in association with a pharmaceutically acceptable excipient or carrier.

The compound for treating, preventing or slowing the progression of neoplasia and cancer, and infection diseases comprises the examples, isomers, stereoisomers, prodrugs, pharmaceutically acceptable salts, complex salts, solvates of the compounds or pharmaceutical formulations and carriers.

The compound for treating, preventing or slowing the progression of neoplasia and cancer, and infection diseases by virus, bacterial or fungi, including bacterial infections and fungal infections of the drug application, which comprises administration together with at least one known chemotherapeutic agent selected from the group consisting of antibacterial and antifungal drugs to a patient in need of such treatment.

The method for treating cancer, comprising: administration to a compound of the claim 1 in the range of 0.001 mg/kg-250 mg/kg, a pharmaceutically acceptable salt or prodrug from thereof; a cancer is selected from the lung cancer, stomach cancer, colon cancer, small cell lung cancer, thyroid cancer, esophageal cancer, pancreatic cancer, endometrial cancer, adrenal cortical carcinoma, head and neck cancer, osteogenic sarcoma, breast cancer, ovarian cancer, Vail Williams tumors, cervical tumors, testicular cancer, genitourinary cancer, skin cancer, renal cell carcinoma, bladder cancer, primary brain cancer, prostate cancer, soft tissue sarcoma, neuroblastoma, rhabdomyosarcoma, Kaposi sarcoma, malignant melanoma, malignant pancreatic islet tumors, non-Hodgkin's lymphoma, malignant melanoma, multiple myeloma, neuroblastoma, malignant carcinoid cancer, choriocarcinoma, acute and chronic lymphocytic leukemia, primary macroglobulinemia, chronic my eloid leukemia, chronic lymphocytic leukemia, acute myeloid leukemia, hairy cell leukemia, mycosis fungoides, malignant hypercalcemia, cervical hyperplasia, or Hodgkin's disease.

The method according to claims 7, wherein said compounds is administered together with at least one known cancer, chemotherapeutic and immune agent selected from cyclophosphamide, vincristine, busulfan, vinblastine, cisplatin, carboplatin, mitomycin C, doxorubicin, colchicine, etoposide, paclitaxel, docetaxel, camptothecin, topotecan, arsenic trioxide, 5-azacytidine, 5-fluorouracil, methotrexate, 5-fluoro-2-deoxyuridine, hydroxyurea, thioguanine, melphalan, chlorambucil, ifosfamide, mitoguazone, epirubicin, aclarubicin, bleomycin, mitoxantrone, elliptinium acetate, fludarabine, octreotide, retinoic acid, tamoxifen, doxazosin, terazosin tamsulosin, tamsulosin, fluorine pyridinoline, lovastatin, simvastatin, pravastatin, fluvastatin, atorvastatin, atorvastatin, amprenavir, abacavir, flavonoids pyridinoline, ritonavir, saquinavir, rofecoxib, alanosine, retinal, tretinoin tocoferil, 13-cis-retinoic acid, 9-cis-retinoic acid, α-difluoro-methyl ornithine, fenretinide, N-4-carboxyphenyl retinamide, genistein, ara-C, CB-64D, CB-184, ILX23-7553, lactacystin, MG-132, PS-341, Glcevec, ZD1839 (IRessa), SH268, Herceptin, Rituxan, Gamcitabine, ABT-378, AG1776, BMS-232, 632, CEP2563, SU6668, EMD121974, R115777, SCH-66336, L-778, 123, BAL9611, TAN-1813, UCN-01, Roscovitine, Olonoucine, Valecoxib.

The administration may be by oral route, parenteral, subcutaneous, intravenous, intramuscular, intra-peritoneal, transdermal, buccal, intrathecal, intracranial, intranasal or topical routes.

### BRIEF DESCRIPTION OF THE DRAWINGS

FIG. 1. shows the inhibition growth of sarcoma S₁₈₀ anatomy (Kunming mice inoculated with S₁₈₀ administered 7 days) of 10 compounds.

### SYNTHESIS AND PREPARATION

### Preparation Example Synthesis Example

The following Examples illustrate the present invention, if not mentioned, the chemical preparation of the reaction at room temperature and IR (KBr, cm⁻¹):

### General Method A (Carboxyl Esterification)

To a mixture of acidic compound 3.20 mmol, 1-ethyl-3-(3-dimethyl-aminopropyl)carbodiimide (EDCI) 1.23 mg (6.40 mmol), and 4-dimethylaminopyridine (DMAP) 0.78 g (6.40 mmol) in 45 ml THF were added dropwise methanol 1.02 g. The mixture was stirred until the reaction was complete according to thin layer chromatography. Subsequently, the reaction mixture was poured in to 100 ml of saline water and extracted with ethyl acetate (3×). The organic phase was evaporated under vacuum and title compound was obtained from the residue by means of flash chromatography (SiO₂).

### General Method B (Carboxyl Amidation)

To a mixture of acidic compound 360 mg (0.58 mmol), EDCI 221 mg (1.16 mmol), DMAP 141 mg (1.16 mmol) and HOBT 78 mg (0.58 mmol) in 5 ml DMF, were added dropwise amine compound 1.16 mmol. The mixture was stirred until the reaction was complete according to thin layer chromatography. Subsequently, the reaction mixture was poured into 50 ml of saline water and extracted with ethyl acetate (3×). The organic phase was evaporated under vacuum and title compound was obtained from the residue by means of flash chromatography (SiO₂).

### General Method C (Hydrogenation)

To a mixture of Pd/C 1.0 g and methanol 20 ml were added nitro-compound 5 mmol. The mixture was hydrogened under stiffing for 7 h. until the reaction was complete according to thin layer chromatogramphy. Subsequently, the organic phase was evaporated under vacuum and then ether was added into the mixture. The solid was filtered and title compound was obtained.

All structures of examples list in Table 1

### Example 1

7-hydroxy-5-[4-(trifluoromethyl)phenyl]pyrazolo[1,5-a]pyrimidino-3-carbonitrile (4.00 g, 14.98 mmol) was added to a solution of trichloroacetic phosphorus oxychloride 13 ml, refluxed 1 h. The reaction mixture was poured into ice water, and filtered to give chlorides; the chlorides (1.60 g, 4.93 mmol), amantadine (1.04 g, 6.90 mmol) and potassium carbonate (2.04 g, 14.78 mmol), DMSO 6 ml, 60° C. reaction 3 h. The title compound was separated by column chromatography; IR: 3460, 2918, 1623, 1593, 1455, 1266, 1161, 1066; ¹H-NMR (300 MHz, DMSO-d6): δ 8.69 (s, 1H), 8.43 (d, J=8.4 Hz, 2H), 7.89 (d, J=8.4 Hz, 2H), 7.11 (s, 1H), 7.02 (s, 1H), 2.18 (s, 9H), 1.80 (d, J=12 Hz, 3H), 1.70 (d, J=12 Hz, 3H).

### Example 2

to 100 ml eggplant-shaped flask and 2-(5-nitro-1,3-dioxoisoindol-2-yl)acetic acid 7.72 g, DMF 60 ml, amino acid 3.30 g, 150° C., stirred for 5 h. The title compound was separated by column chromatography; IR: 3248, 2918, 2836, 1773, 1691, 1638, 1623, 1560, 1515, 1430, 1412, 1354, 1281, 1251, 1233, 1155, 1114, 958, 777.

### Example 3

the title compound was synthesized by general method C with starting meteriel, N-(adamant-1-yl)-2-(5-nitro-1,3-dioxo-isoindol-2-yl)acetamide; IR: 3443, 3239, 2908, 1766, 1688, 1642, 1619, 1505, 1420, 1291, 1268, 1243, 1160, 1104; ¹H-NMR (300 MHz, DMSO-d₆): δ 7.65 (s, 1H), 7.47 (d, J=7.8 Hz, 1H), 6.90 (d, J=1.8 Hz, 1H), 6.79 (dd, J=1.8 Hz, J=8.4 Hz, 1H), 6.47 (s, J=7.8 Hz, 2H), 4.01 (s, 2H), 1.98 (s, 3H), 1.88 (br, 6H), 1.58 (m, 6H,).

### Example 4

to a mixture of hexahydro-4,7-epoxy-isobenzofuran-1,3-one 0.25 g (1.5 mmol), triethylamine 0.3 g (3.0 mmol) and toluene 12 ml was added N-(adamant-1-yl)-2-aminopropionamide 0.5 g (1.5 mmol), refluxed 2 h. The title compound was separated by column chromatography;

IR: 3396, 2908, 2852, 1776, 1709, 1666; ¹H-NMR (300 Hz, DMSO-d6): δ 6.68 (s, 1H), 4.68 (s, 2H), 4.39 (q, J=3.6 Hz, 1H), 3.01 (s, 2H), 1.97 (s, 3H), 1.85 (s, 6H), 1.63 (s, 4H), 1.58 (s, 6H), 1.32 (d, J=3.6 Hz, 3H).

### Example 5

to a mixture of hexahydro-4,7-epoxy-isoindole-1,3-dione 0.25 g (1.5 mmol), triethylamine 0.3 g (3.0 mmol) and toluene 12 ml was added N-(1-azaadamantane-4-yl)-2-(2,2,2-trifluoroacetyl)propionamide 0.48 g (1.5 mmol), refluxed for 2 h. The title compound was separated by column chromatography; IR: 3342, 2968, 1786, 1719, 1662; ¹H-NMR (300 Hz, DMSO-d6): δ 6.78 (s, 1H), 4.54 (s, 2H), 4.32 (q, J=3.6 Hz, 1H), 3.03 (s, 2H), 2.25 (m, 6H), 1.94 (s, 2H), 1.62 (s, 4H), 1.46 (s, 6H), 1.21 (d, J=3.6 Hz, 3H).

### Example 6

to a mixture of 3a,4,7,7a-tetrahydro-4,7-epoxy-isobenzofuran-1,3-dione 0.25 g (1.5 mmol), glycine 0.13 g (1.5 mmol) and DMF 12 ml was added 1-azaadamantyl-3-amine 0.23 g (1.5 mmol), refluxed for 2 h. The title compound was separated by column chromatography; IR: 3432, 2978, 2896, 1795, 1721, 1656.

### Example 7

to a mixture of trifluoroacetic acid and 10 ml ethyl ether 20 ml, potassium hydro-oxide solution, 10 ml (40%) was added N-(1-azaadamantyl-3-yl)-2-(1,3-dioxo-3a,4,7,7a-tetrahydro-1H-4,7-epoxy-isoindole-2(3H)-yl)acetamide 3.57 g (10 mmol), stirred for 3 h. The title compound was separated by column chromatography; IR: 3452, 2905, 2886, 1772, 1735, 1622, 1534, 1452, 1366; ¹H-NMR (300 Hz, DMSO-d6): δ 6.66 (s, 1H), 4.45 (s, 2H), 4.44 (q, J=3.6 Hz, 1H), 3.58 (br, 1H), 3.23 (s, 2H), 2.32 (m, 6H), 1.84 (s, 2H), 1.80 (m, 4H), 1.45 (s, 6H).

### Example 8

The title compound was synthesized by general method B with starting meteriel, N-(adamant-1-yl)-2-(5-nitro-1,3-dioxo-isoindol-2-yl)acetamide; IR: 3288, 2928, 1768, 1699, 1648, 1622, 1555, 1432, 1359, 1289, 1234.

### Example 9

the title compound was synthesized by general method C with starting meterial N-(1-azaadamantyl-3-yl)-2-(5-nitro-1,3-dioxo-isoindol-2-yl)acetamide; IR: 3433, 3269, 2928, 1769, 1698, 1644, 1620, 1543, 1420, 1287, 1233, 1150, 1103; ¹H-NMR (300 Hz, DMSO-d6): δ 8.03 (s, 1H), 7.97 (d, J=7.8 Hz, 1H), 7.27 (s, 1H), 6.79 (d, J=7.8, 1H), 6.59 (s, 2H), 4.81 (m, 3H), 2.33 (m, 2H), 2.20 (br, 4H), 2.03 (m, 2H), 1.56 (m, 2H), 1.41 (m, 1H), 1.18 (m, 2H).

### Example 10

to a solution of phosphorus oxychloride 4 ml was added 3-(2,4-dichlorophenyl)-2-methyl-6-phenyl-pyrazolo[1,5-a]pyrimidine-5,7-diol (1.00 g, 2.59 mmol), refluxed for 8 h. The title compound was separated by column chromatography; IR: 3426, 2961, 1615, 1551, 1384, 198, 1133; to a mixture of amantadine (2.15 g, 1.41 mmol), potassium carbonate (0.50 g, 3.54 mmol) and 10 Ml DMSO was added the above product (0.50 g, 1.18 mmol) 60° C. stirred 15 h. The title compound was separated by column chromatography; IR: 3435, 3302, 2910, 1615, 1517, 1456, 1406, 1357, 1300, 1261, 1186, 1072; ¹H-NMR (300 Hz, DMSO-d6): δ 7.52 (d, J=2.4 Hz, 1H), 7.49 (t, J=7.2 Hz, 2H), 7.45 (t, J=7.2 Hz, 1H), 7.39 (dd, J=7.2 Hz, J=1.2 Hz, 2H), 7.36 (d, J=8.4 Hz, 1H), 7.32 (dd, J=7.2 Hz, 2.4 Hz, 1H), 5.61 (s, 1H), 2.40 (s, 3H), 1.97 (s, 3H), 1.76 (s, 6H), 1.56 (d, J=12H z, 3H), 1.49 (d, J=12 Hz, 3H).

### Example 11

to a mixture of anhydrous potassium carbonate (0.56 g, 4.09 mmol), KI (0.23 g, 1.36 mmol), N-adamantyl-2-chloroacetamide (0.30 g, 1.36 mmol) and 3 ml DMF was added 4,5-diphenyl-1H-imidazole 0.30 g (1.36 mmol) 50° C. stirred for 10 h The title compound was separated by column chromatography; IR: 3435, 2909, 1672, 1553; ¹H-NMR (300 Hz, DMSO-d6): δ 7.71 (s, 1H), 7.46 (s, 1H), 7.45 (s, 3H), 7.35 (d, 2H), 7.26 (m, 2H), 7.17 (t, 2H), 7.10 (t, 1H), 4.37 (s, 2H), 1.97 (s, 3H), 1.81 (s, 6H), 1.58 (q, 6H).

### Example 12

the title compound was synthesized by general method A with starting meterials, 2-(2-(4,5-diphenyl-1H-imidazol-1-yl))-N-(dihydroxyethyl)acetamide and adamantan-1-carboxylic acid; IR: 3449, 2920, 1712, 1660; ¹H-NMR (300 Hz, DMSO-d6): δ 8.05 (br, 1H), 7.74 (s, 1H), 7.44 (t, J=3 Hz, 3H), 7.35 (d, J=7.8 Hz, 2H), 7.26 (m, 2H), 7.17 (t, J=7.8 Hz, 2H), 7.10 (t, J=7.2 Hz, 1H), 4.45 (s, 2H), 3.90 (t, J=5.4 Hz, 2H), 3.23 (q, J=5.4 Hz, 2H), 1.93 (s, 3H), 1.76 (s, 6H), 1.62 (dd, J=12 Hz, J=24 Hz, 6H).

### Example 13

the title compound was synthesized by general method A with starting meterials 4,5-diphenyl-1H-imidazol-2-yl amine and adamantan-1-carboxylic acid; IR: 2905, 1654, 1384; ¹H-NMR (300 Hz, DMSO-d6): δ 11.67 (s, 1H), 10.59 (s, 1H), 7.45 (d, J=7.2 Hz, 2H), 7.40 (d, J=7.2 Hz, 2H), 7.36 (t, J=7.8 Hz, 2H), 7.28 (d, J=7.2 Hz, 1H), 7.25 (t, J=7.8 Hz, 2H), 7.18 (t, J=7.8 Hz, 1H), 2.00 (s, 3H), 2.19 (s, 6H), 1.69 (s, 6H).

### Example 14

the title compound was synthesized by general method B with starting meterial, 3-(2,4-dichlorophenyl)-2,7-dimethyl-pyrazolo[1,5-a]pyrimidine-6-carboxylic acid and adamantan-1-amine; IR: 3447, 3079, 2908, 2847, 1633, 1593, 1511, 1529, 1497, 1455, 1383; ¹H-NMR (300 Hz, DMSO-d6): δ 8.39 (s, 1H), 8.06 (s, 1H), 7.77 (d, J=2.4 Hz, 1H), 7.52 (dd, J=7.8 Hz, J=2.4 Hz, 1H,), 7.46 (d, J=7.8 Hz, 1H), 2.80 (s, 3H), 2.36 (s, 3H), 2.06 (s, 10H), 1.65 (s, 6H).

### Example 15

the title compound was synthesized by general method B with starting meterial, 7-amino-3-(2,4-dichlorophenyl)-2-methyl-pyrazolo[1,5-a]pyrimidine-6-methyl cyanide and adamantan-1-acid; IR: 3326, 2980, 2851, 2220, 1735, 1618, 1596, 1532, 1492, 1463, 1380, 1364, 1298, 1207, 1173, 1100, 1064; ¹H-NMR (300 Hz, DMSO-d6): δ 10.91 (s, 1H), 8.71 (s, 1H), 7.80 (d, J=1.2 Hz, 1H), 7.55 (m, 1H), 7.51 (m, 1H), 2.34 (s, 3H), 2.07 (br, 3H), 2.02 (br, 6H), 1.73 (br, 6H).

### Example 16

the title compound was synthesized by general method B with starting meterials, 5-(2-aminoethylamino)-8H-phthalazino[1,2-b]quinazolin-8-one and adamantan-1-acid; IR: 3329, 3068, 2901, 2849, 1675, 1630, 1530, 1485, 1467, 1449, 1384, 1342, 1316, 1278, 1237, 1178, 1139, 1126, 1036; ¹H-NMR (300 Hz, DMSO-d6): δ 8.89 (dd, 1H, J=1.2 Hz, J=7.8 Hz), 8.25 (dd, J=1.2 Hz, J=7.8 Hz, 1H), 8.20 (d, J=8.4 Hz, 1H), 7.97 (m, 2H), 7.84 (m, 1H), 7.81 (d, J=7.80 Hz, 1H), 7.64 (m, 2H), 7.51 (m, 1H), 3.57 (m, 2H), 3.45 (m, 2H), 1.87 (br, 3H), 1.73 (br, 6H), 1.58 (m, 6H).

### Example 17

to a mixture of amantadine 0.54 g (3.60 mmol) potassium carbonate 0.83 g (6.00 mmol) and THF20 ml was added 7-chloro-3-(2,4-dichlorophenyl)-2,6-dimethyl-5-(4-trifluoro-methylphenyl)-pyrazolo[1,5-a]pyrimidine 1.41 g (3.00 mmol), 65° C. for 20 h. The reaction mixture was poured into ice water and filtered to give target product; IR: 3315, 2912, 1616, 1527, 1493, 1459, 1404, 1376, 1356, 1321, 1272, 1259, 1186, 1166, 1126, 1081; ¹H-NMR (300 Hz, DMSO-d6): δ 7.72 (d, J=8.4 Hz, 2H), 7.68 (d, J=8.4 Hz, 2H), 7.52 (d, J=2.4 Hz, 1H,), 7.36 (d, J=7.8 Hz, 1H), 7.28 (dd, J=1.8 Hz, J=7.8 Hz, 1H), 5.45 (s, 1H), 2.42 (s, 3H), 2.32 (s, 3H), 2.19 (br, 3H), 2.10 (br, 6H), 1.72 (br, 6H).

### Example 18

to a mixture of sodium hydride 0.42 g (60%), THF10 ml, dimethyl ethanolamine 0.81 g was added N-1-adamantyl alkyl-2-chloroacetamide 1.60 g (7.00 mmol) in THF 40 ml, refluxed for 3 h. The reaction mixture was poured into ice water and filtered to give the title product; IR: 3414, 3214, 3026, 2908, 2851, 1671, 1535, 1513, 1454, 1384, 1360, 1344, 1297, 1251, 1224, 1129, 1116, 1098, 1051, 996, 970, 859, 815; ¹H-NMR (300 Hz, DMSO-d6): δ 10.74 (br, 1H), 7.33 (s, 1H), 3.83 (s, 2H), 3.72 (m, 2H), 3.24 (m, 2H), 2.76 (s, 6H), 1.95 (m, 9H), 1.59 (br, 6H).

### Example 19

the title compound was synthesized by general method B and D with starting meterials 4-((3,4,5-acetoxy-6-(acetoxymethyl)-tetrahydro-2H-pyran-2-yl)oxy)benzoic acid and adamantan-1-amine; IR: 3429, 2914, 2850, 1637, 1608, 1500; ¹H-NMR (300 Hz, DMSO-d6): δ7.76 (d, J=8.7 Hz, 2H), 7.44 (s, 1H), 7.01 (d, J=8.7 Hz, 2H), 5.75 (s, 1H), 4.98 (d, J=3.6 Hz, 1H), 4.67 (d, J=7.5 Hz, 1H), 4.50 (t, J=5.4 Hz, 1H), 3.93 (d, J=3.0 Hz, 1H), 3.68 (m, 2H), 2.06 (s, 9H), 1.65 (s, 6H), 2.05-1.06 (br, 4H).

### Example 20

to a mixture of Boc-L-alanine 6.3 g (33 mmol), EDCI 9.5 g (49 mmol), DMAP 4 g (32 mmol), HOBT 4.4 g (32 mmol) and DMF 120 ml was added amantadine hydrochloride 12.4 g (21 mmol), reacted for 3 h. The reaction mixture was poured into ice water, and filtered to give the title product; IR: 3445, 3100, 2915, 1670.

### Example 21

to a mixture of hexahydro-4,7-epoxyisoindole-1,3-one 0.25 g (1.5 mmol), triethylamine 0.3 g (3.0 mmol) and toluene 12 ml was added N-(adamant-1-yl)-2-aminopropionamide 0.5 g (1.5 mmol), refluxed 2 h. The title compound was separated by column chromatography; IR: 3396, 2908, 2852, 1776, 1709; ¹H-NMR (300 Hz, DMSO-d6): δ 6.68 (s, 1H), 4.68 (s, 2H), 4.39 (q, J=3.6 Hz, 1H), 3.01 (s, 2H), 1.97 (s, 3H), 1.85 (s, 6H), 1.63 (s, 4H), 1.58 (s, 6H), 1.32 (d, J=3.6 Hz, 3H).

### Example 22

to a mixture of 5,6-dibromohexahydro-4,7-epoxyisoindole-1,3-dione 0.48 g (1.5 mmol), triethylamine 0.3 g (3.0 mmol) and toluene 10 ml was added N-(adamant-1-yl)-2-aminopropionamide 0.5 g (1.5 mmol), refluxed for 3 h. The title compound was separated by column chromatography; IR: 3406, 2996, 2909, 2850, 1782, 1709, 1678; ¹H-NMR (300 Hz, DMSO-d6): δ 5.32 (s, 1H), 4.99 (t, J=5.4 Hz, 1H), 4.92 (d, J=10.5 Hz, 1H), 4.62 (q, J=7.2 Hz, 1H), 4.39 (m, 1H), 3.99 (t, J=3.0 Hz, 1H), 3.82 (t, J=7.5 Hz, 1H), 3.12 (t, J=6.6 Hz, 1H), 2.07 (s, 3H), 1.95 (m, 6H), 1.70-1.66 (m, 8H), 1.52 (d, J=7.2 Hz, 3H).

### Example 23

to a mixture of phthalic anhydride, 0.12 g (0.8 mmol), triethylamine 0.16 g (1.6 mmol) and toluene 10 ml was added N-(adamant-1-yl)-2-aminopropionamide 0.27 g (0.8 mmol), refluxed for 3 h. The title compound was separated by column chromatography; IR: 3304, 3082, 2907, 2859, 1779, 1714, 1656, 1613, 1555; ¹H-NMR (300 Hz, DMSO-d6): δ 7.84 (m, 4H), 7.32 (s, 1H), 4.64 (q, J=3.6 Hz, 1H), 1.97 (s, 3H), 1.89 (s, 6H), 1.56 (s, 6H), 1.54 (d, J=3.9 Hz, 3H).

### Example 24

to a mixture of 4-nitrophthalic anhydride 0.3 g (1.5 mmol), triethylamine 0.3 g (3.0 mmol) and toluene 10 ml was added N-(adamant-1-yl)-2-aminopropionamide 0.5 g (1.5 mmol), refluxed for 3 h. The reaction solution was concentrated and title compound was purified by column; IR: 3318, 3080, 2908, 2849, 1781, 1722, 1641, 1530, 1452, 1346; ¹H-NMR (300 Hz, DMSO-d6): δ 8.62 (dd, J=7.2 Hz, 1H), 8.49 (d, J=1.8 Hz, 1H), 8.12 (d, J=8.4 Hz, 1H), 4.68 (q, J=7.2 Hz, 1H), 1.97 (s, 3H), 1.89 (s, 6H), 1.60 (s, 6H), 1.54 (d, J=7.2 Hz, 3H).

### Example 25

the title compound was synthesized by general method C with starting materials nitro compound; IR: 3440, 2907, 2857, 1755, 1698; ¹H-NMR (300 Hz, DMSO-d6): δ 7.58 (d, J=7.8 Hz, 1H), 7.00 (s, 1H), 6.82 (d, J=7.8 Hz, 1H), 5.68 (s, 1H), 4.75 (t, J=6.6 Hz, 1H), 4.49 (s, 2H), 2.06 (s, 3H), 1.99 (s, 6H), 1.63 (m, 9H).

### Example 26

to a solution of acetone 50 ml was added 1,3-dibromoadamantane 5.0 g (17 mmol), refluxed for 3 h, the filtrate was allowed to stand overnight to obtain the white crystal target product; IR: 3220, 2934, 2851, 1028.

### Example 27

to a mixture of 5-chloro-4-nitrobenzoic acid 1.5 g (7.5 mmol), EDCI 2.3 g (12.0 mmol) and DMAP 0.7 g (5.7 mmol) was added adamantanediol 0.5 g (3.0 mmol), stirred for 2 h. The reaction solution was concentrated and the title compound was purified by column chromatography; IR: 3559, 3272, 3056, 2920, 2851, 1731, 1714, 1600, 1528; ¹H-NMR (300 Hz, DMSO-d6): δ 8.57 (d, J=2.4 Hz, 1H), 8.23 (q, J=2.4 Hz, 1H), 2.42-1.55 (m, 15H).

### Example 28

to a mixture of lithium aluminum hydride 0.02 mmol in THF was added 1-azaadamantane-4-one 0.02 mmol, stirred for 30 min and the title compound was separated by column chromatography; ¹H-NMR (300 Hz, DMSO-d6): δ3.15 (m, 1H), 2.23 (m, 6H), 1.75 (m, 3H), 1.49 (m, 2H), 124 (m, 2H).

### Example 29

to a mixture of triphenylphosphonium bromide 2.00 g (5.6 mmol) in 8 ml of THF and butyl lithium 3 ml (7.5 mmol) was added 1-aza-adamantyl alkyl-4-one 500 mg (3.3 mmol) in 15 ml of THF, reacted for 2 h. The title product was obtained by filtration of the residue; ¹H-NMR (300 Hz, CDCl₃): δ 4.49 (s, 2H), 3.00-3.21 (m, 4H), 3.12 (s, 2H), 2.27 (s, 2H), 1.85-2.04 (m, 4H), 1.63 (s, 1H).

### Example 30

to a mixture of p-toluenesulfonyl methyl isocyanide 6.38 g (32.3 mmol), potassium tert-butoxide 6.70 g (59.7 mmol), 1,2-ethylene glycol dimethyl ether 87 ml and ethanol 3.2 ml was added 1-azaadamantane-4-one 3.76 g (24.9 mmol), 40° C. stirred for 0.5 h. The title compound was separated by column chromatography; ¹H-NMR (300 MHz, methanol-d4): δ2.05-2.46 (m, 6H), 1.21-1.72 (m, 7H).

### Example 31

to a mixture of concentrated hydrochloric acid and acetic acid mixture of 4 ml (1:1) was added 1-aza-adamantane-4-acetonitrile 136 mg (0.84 mmol), 110° C. stirred for 14 h. The title compound was separated by column chromatography; IR: 3347, 2989, 2961, 2850, 1679, 1560, 1433, 1249, 1035; ¹H-NMR (300 MHz, DMSO-d6): δ 10.32 (s, 1H), 1.22-2.36 (m, 14H).

### Example 32

to a mixture of lithium aluminum hydride (3.80 mmol) in THF 3.8 ml was added of 1-azaadamantane-4-acetonitrile 0.41 g (2.53 mmol) in THF 6 ml, refluxed 2 h, cooled and then water 144 ml and sodium hydroxide 144 ml (15%) were added. The precipitated aluminum salts was removed by filtration and the filtrate was concentrated to give the title product; IR: 3421, 2926, 2884, 1524, 1431; ¹H-NMR (300 MHz, DMSO-d6): δ 5.31 (br, 2H), 2.64 (d, J=3.6 Hz, 2H), 2.205-2.36 (m, 6H), 1.05-1.62 (m, 8H).

### Example 33

to a mixture of dicyclohexyl carbodiimide 230 mg (1.2 mmol) and DMAP 147 mg (1.2 mmol) in ethanol 3 ml was added 1-aza-adamantane carboxylic acid 181 mg (1 mmol), stirred for 3 h. The filtrate was concentrated to give the title product; IR: 3386, 2988, 1734, 1438, 1356, 1229, 1059; ¹H-NMR (300 MHz, DMSO-d6): δ 3.86 (q, J=3.6 Hz, 2H), 1.13-2.43 (m, 17H).

### Example 34

to a mixture of lithium aluminum hydride (1 mmol) in THF was added dropwise a suspension of 1-aza-adamantane-ethyl 209 mg (1 mmol) in THF, stirred for 1 h and the reaction mixture was added water and 5% hydrogen aqueous solution of sodium oxide. The reaction solution was concentrated under reduced pressure to obtain the title product; IR: 3434, 2938, 1434, 1268; ¹H-NMR (300 MHz, DMSO-d6): δ 3.49 (d, J=3.6 Hz, 2H), 2.05-2.36 (m, 7H), 1.35-1.66 (m, 8H).

### Example 35

to a mixture of sodium borohydride 9.08 g (240 mmol) and water 45 ml was added the 2,4,6-trinitrophenol 12.0 g (52 mmol) and sodium hydroxide 300 ml (1.5%), stirred 20 min. The concentrated phosphoric acid solution was added to adjust PH=5.0, the precipitate was filtered to obtain 1,3,5-trinitro-1,3,5-(hydroxymethyl)cyclohexyl alkyl and then water 400 ml was added and stirred for 1 h, and the precipitate was filtered to obtain the target product Mp; IR: 1540, 1345; ¹H-NMR (300 MHz, DMSO-d6): δ 3.42 (s, 6H), 3.00 (d, J=13 Hz, 3H) 2.80 (d, J=13 Hz, 3H).

### Example 36

to a mixture of isopropanol 100 ml and Swiss mud Nickel 0.1 g was added 3,5,7-triamino-1-azaadamantane 5.0 g (27.4 mmol), stirred for 4 h. The title compound was separated by column chromatography; IR: 3350-3010; ¹H-NMR (300 MHz, DMSO-d6): δ 2.65 (s, 6H), 1.59 (s, 6H), 2.24 ppm (s, 2H).

### Example 37

to a solution of acetic anhydride 5.0 ml was added 3,5,7-tribromo-1-aza-adamantane 0.55 g (3 mmol), refluxed for 3 h. The title compound was separated by column chromatography; IR: 3240, 3040, 1740, 1640; ¹H-NMR (300 MHz, DMSO-d6): δ 2.54-2.81 (m, 6H), 2.12-2.40 (m, 6H).

### Example 38

to a mixture of phloroglucinol compound 2.46 g (10 mmol) and methanol 30 ml was added hexamethylenetetramine 1.40 g (10 mmol), refluxed for 15 h. The reaction solution was concentrated to give a white solid title product; IR: 3421, 2908, 1656, 1632, 1508, 1321; ¹H-NMR (300 MHz, DMSO-d6): δ 5.32-5.65 (m, 9H), 2.88 (s, 6H), 2.52 (s, 6H).

### Example 39

to a mixture of methenamine 1.40 g (10 mmol) and methanol 30 ml was added 2,4,6-trimethylcyclohexane-1,3-dione 1.54 g (10 mmol), refluxed for 5 h. The reaction solution was concentrated and recrystallized from ethanol to give a white title product; IR: 2970, 1720, 1685, 1455, 1375, 1330, 1205, 1125; ¹H-NMR (300 Hz, CDCl₃): δ 3.45-2.75 (m, 6H), 1.70 (s, 2H), 1.16 (s, 3H), 1.05 (s, 6H).

### Example 40

to a solution of borane in THF 14.80 ml (14.8 mmol) was added 3-allyl-7-(methoxymethyl)-3-boronbicyclo[3.3.1]non-6-ene 2.86 g (14.8 mmol), refluxed for 1 h. The reaction solution was concentrated to give a white title product; 1H-NMR (300 Hz, CDCl₃): δ 3.34 (t, J) 6.8 (4H), 2.74 (br s, 3H), 2.00 (m, 6H), 1.01 (m, 10H).

### Example 41

to a mixture of potassium tertbutoxide 0.56 g (5 mmol) in THF 20 ml and hydrazine (0.32 g, 10 mmol) in THF 10 ml was added 1-azaadamantane-4-one 1.51 g (10 mmol), stirred 1.5 h. The title compound was separated by column chromatography; IR: 2986, 1642, 1358, 1264; ¹H-NMR (300 MHz, DMSO-d6): δ 2.12-2.56 (m, 6H), 1.32-1.58 (m, 7H). To a mixture of methanol 30 ml and hydrogen peroxide 10 ml (35%) was added 1-azaadamantane 1.37 g (10 mmol), stirred for 6 h. The title compound was separated by column chromatography; IR: 2988, 1686, 1324, 1258, 1105; ¹H-NMR (300 MHz, DMSO-d6): δ 3.58 (t, J=3.6 Hz, 1H), 2.82 (m, 1H), 1.21-2.3 (m, 10H).

### Example 42

to a mixture of pyridine 1 ml and sulfur phosphide 14 mg (0.03 mmol) was added 1-azaadamantane-1-oxide 30 mg (0.2 mmol), refluxed for 14 h. The title compound was separated by column chromatography; ¹H-NMR (300 Hz, CDCl₃): δ1.84 (bs, 1H), 2.16-2.35 (m, 4H), 2.58 (s, 2H), 3.25-3.56 (m, 6H).

### Example 43

to a mixture of acetone 50 ml and chromic acid solution containing sulfuric acid (8 N) was added 2-oxaadamantane-4-ol 4.0 g (25.9 mmol), stoned for 2 h. The title compound was separated by column chromatography; ¹H NMR (300 Hz, CDCl₃): δ 4.10-3.99 (m, 2H), 2.75-1.74 (m, 10H).

### Example 44

to a mixture of lithium aluminum hydrogenation 1.0 g (26 mmol) in diethyl ether solution of 100 ml was added bicyclo[3.3.1]non-6-en-3-one 5 g (36.7 mmol), refluxed for 8 h. The title compound was separated by column chromatography; IR: 3369, 2980, 2887, 1631, 1356, 1294; ¹H-NMR (300 MHz, DMSO-d6): δ 3.58 (m, 1H), 2.78 (m, 1H), 2.30 (m, 1H), 2.0 (m, 2H), 1.42-1.75 (m, 7H).

### Example 45

to a mixture of methanol 30 ml and sodium borohydride 38 mg (1 mmol) was added a protected glycol bicyclo[3.3.1]nonane-3,7,9-trione 210 mg (1 mmol), stirred for 3 h and added with 1 ml HCl (6 N) in 1,4-dioxane to obtain the title product; IR: 3431, 2988, 1664, 1356, 1201; ¹H-NMR (300 MHz, DMSO-d6): δ 3.21 (m, 1H), 1.54-2.36 (m, 11H).

### Example 46

to a mixture of ammonia solution in methanol 7 N and 5% palladium on carbon was added 1-hydroxy-2-adamantyl-oxa-6-one 1M, stirred for 12 h and the reaction solution was filtered to give the title product by evaporation; IR: 3432, 3421, 3358, 2864, 1357; ¹H-NMR (300M Hz, DMSO-d₆): δ 5.11 (s, 2H), 3.52 (s, 1H), 2.58 (t, J=2.1 Hz, 1H), 1.36-2.02 (m, 11H).

### Example 47

to a mixture of sodium borohydride 42 mg (1.1 mmol) and methanol solution of 50 ml was added bicyclo[3.3.1]nonane-3,7-dione 132 mg (1.0 mmol), refluxed for 3 h. The reaction solution was filtered to obtain the title product: IR: 3345, 2986, 1432, 1350, 1114; ¹H-NMR (300 MHz, DMSO-d6): δ 3.45 (s, 1H), 2.21 (br, 1H), 1.12-1.64 (m, 12H).

### Example 48

to a mixture of methenamine 155 mg (1.1 mmol) and methanol 200 ml was added was added trimethyl phloroglucinol 168 mg (1 mmol), refluxed for 48 h. The reaction solution was filtered to obtain the title product: IR: 2987, 2975, 2934, 1736, 1688; ¹H-NMR (300 MHz, CDCl₃): δ 1H 1.25 (m, 9H), 3.41 (s, 6H).

### Example 49

to a mixture of peroxide 85%, m-chloroperbenzoic acid 4.04 g (0.02 mol), methylene chloride 40 ml was added N-(bicyclo[3.3.1]non-6-en-3-yl)benzamide 4.8 g (0.02 mol) in dichloromethane 40 ml, stirred for 18 h. The title compound was separated by column chromatography; IR: 3320, 2930, 2850, 1590, 1570, 1445, 1375, 1080, 1025, 970, 920, 790, 735, 700; ¹H-NMR (CDCl₃): δ 1.18-2.54 (m, 10H), 3.45 (s, 1H), 3.80 (m, 2H), 4.75 (m, 1H), 7.34 (s, 5H).

### Example 50

to a mixture of diborane THF solution 20 ml (1 M) was added 2-hydroxyl adamantamine benzoylamide 2.57 g (10 mmol) in THF 25 ml, refluxed for 3 h. The title compound was separated by column chromatography; IR: 3340, 2930, 2850, 1500, 1455, 1360, 1150, 1080, 1035, 1000, 740, 700; ¹H-NMR (CDCl₃): δ 1.18-2.33 (m, 11H), 2.67 (m, 2H), 3.81 (s, 2H), 4.00 (m, 1H), 7.24 (br, s, 5H).

### Example 51

to a mixture of palladium on carbon catalyst 100 mg of 5% in 50 ml of ethanol was added N-benzyl-2-hydroxyl adamamine 0.73 g (0.003 mol). After the hydrogenation reaction was completed and then the precipitate was filtered to give title product; IR: 3500-3100, 2900, 2850, 1640, 1580, 1460, 1060, 1025.

### Example 52

to a mixture of benzenesulfonyl chloride 1.91 g (0.01 mol) and pyridine 20 ml was added 4-hydroxy-2-azaadamantyl-2-yl)benzophenone 2.57 g (0.01 mol), stirred for 14 days. The title compound was separated by column chromatography; IR: 3010, 2940, 2880, 1640, 1595, 1460, 1420, 1375, 1360, 1290, 1185, 1170; ¹H-NMR (CDCl₃): δ 1.40-2.40 (m, 10H), 2.47 (s, 3H), 3.90 (m, 1H), 4.68 (m, 2H), 710-800 (m, 9H).

### Example 53

to a mixture of chromium trioxide 1.2 g (0.012 mol), (N-benzoyl-2-azaadamantane-4-alcohol) p-toluenesulfonate 0.514 g (0.002 mol) and dichloromethane 40 ml was added pyridine 1.9 g (0.024 mol), stirred for 15 min. The reaction solution was concentrated to obtain pale yellow oily product; IR: 3050, 2925, 2860, 1730, 1620, 1575, 1450, 1410, 1345, 1310, 1245, 1095, 1075, 1055, 1030, 975, 790, 720, 700; ¹H-NMR (CDCl₃): δ 1.77-2.50 (m, 10H), 2.75 (m, 1H), 4.50 (br, 1H), 7.40 (s, 5H).

### Example 54

to a mixture of paraformaldehyde 7.0 g and solution of sulfuric acid 1 L (2%) was added 1,4-dioxaspiro[4,5]aoi-8-yl amine 8.55 g (0.05 mol) in ethanol 20 ml, refluxed for 24 h. The organic phase was separated by column chromatography to obtain the title product; IR: 3332, 2977, 1657, 1408, 1321; ¹H-NMR (300 MHz, DMSO-d6): δ2.38-2.52 (m, 5H), 1.72-2.33 (m, 8H).

### Example 55

to a mixture of 1-N-4-adamantanone 17.1 g (0.113 mol) in ethanol 280 ml and hydroxylamine hydrochloride 11.0 g was added pyridine 9.17 ml (0.113 mol), refluxed for 17 h. The title compound was separated by column chromatography; IR: 3190, 3065, 1662, 1447, 932; ¹H-NMR (300 MHz, CDCl₃): δ 9.32 (s, 1H), 3.47 (s, 1H), 3.36-.3.11 (m, 6H), 2.47 (s, 1H), 2.16 (m, 2H), 2.04 (m, 2H), 1.79 (s, 1H).

### Example 56

to a solution of lithium aluminum hydride in THF 128 ml (0.128 mol) was added dropwise Z-1-azaadamantano cyclic-4-one hydroxylamine 17.7 g (0.107 mol) in THF 300 ml, refluxed for 15 h. Water 20 ml was added dropwise and the reaction solvent was evaporated under reduced pressure to obtain pale yellow titled product; IR: 3445, 3432, 2986, 1432; ¹H-NMR (300 MH z, DMSO-d₆): δ 5.12 (br, 2H), 2.55 (t, J=2.7 Hz, 1H), 2.30 (d, J=3.0 Hz, 6H), 1.36-1.64 (m, 7H).

### Example 57

to a mixture of bicyclo[3.3.1]nonane-3,7-dione 3.0 g (20 mmol) and ammonium acetate 15.4 g (0.199 mol) and methanol 90 ml was added sodium boron 0.868 g (13.8 mol), stirred for 2 days. The title compound was separated by column chromatography; IR: 3345, 3209, 2920, 1432, 1230, 1120; ¹H-NMR (300 MHz, DMSO-d6): δ 3.78 (br, 1H), 2.57 (m, 1H), 1.75 (m, 4H), 1.55 (m, 5H), 1.36 (m, 3H).

### Example 58

to a mixture of thionyl chloride 12.5 ml was added 1-hydroxy-2-adamantyl amine 1.159 g (7.575 mmol), refluxed for 1 h. Methylene chloride 65 ml and sodium hydroxide 50% were added The title compound was separated by column chromatography; IR: 3329, 2923, 2845, 1440, 1342, 1201, 1105; ¹H-NMR (300 MHz, DMSO-d6): δ 2.53 (m, 1H), 1.85 (m, 2H), 60 (m, 4H), 1.49 (m, 3H), 1.36 (m, 3H).

### Example 59

to a ethylene glycol dimethyl ether solution of lithium aluminum hydride containing 0.536 g (14.1 mol) was added 1-chloro-2-adamantylamine 1.495 g (8.717 mmol), refluxed for 2 days and diethyl ether 0.54 ml and aqueous sodium hydroxide solution 0.54 ml 15% were added. The reaction solvent was removed from the extract under reduced pressure to give a brown title product; ¹H-NMR (300 MHz, CDCl₃): δ 1.5-2.2 (m, 12H), 2.45-2.75 (br, 1H), 2.90-3.25 (br, 2H).

### Example 60

to a mixture of ethylene oxide 0.200 g (4.5 mmol) was added 2-N-adamantane 0.551 g (4.02 mmol) in methanol dropwise, stirred for 24 h. The title compound was separated by column chromatography; IR: 3370, 2998, 1060; ¹H-NMR (300 MHz, CDCl₃): δ 1.35-2.30 (m, 12H), 2.65-3.05 (m, 4H), 3.45 (t, J=5 Hz, 2H).

### Example 61

to a solution of thionyl chloride 5.187 g (43.6 mmol) was added dropwise 2-(azaadamantan-2-yl)ethanol 350 mg (1.90 mol) and refluxed for 2.5 h. The title compound was separated by column chromatography; IR: 2560, 2485, 1100; ¹H-NMR (300 MHz, CDCl₃): δ 1.50-3.15 (m, 13H), 3.35-3.82 (m, 4H), 4.19 (t, J=5.5 Hz, 2H).

### Example 62

to a mixture of acetone 1.9 g (0.005 mol), paraformaldehyde 0.94 g (0.025 mol), ammonium acetate 0.77 g (0.01 mol) and ethanol solution 5 ml was added 1,3-bis(3,4,5-trimethoxyphenyl), refluxed 5 h. The reaction mixture was precipitated to give the title product; IR: 2960, 2860, 1710, 1624, 1543, 1456, 1384, 1286, 1211, 1102; ¹H-NMR (300 MHz, CDCl₃): δ 6.50 (s, 4H), 3.83 (s, 18H), 3.43 (s, 2H), 2.84 (m, 8H).

### Example 63

to a mixture of paraformaldehyde 0.94 g (0.025 mol), ammonium acetate 0.77 g (0.01 mol) and ethanol 5 ml was added 1,3-bis(4-nitro-3-hydroxyphenyl)propan-2-one 1.66 g (0.005 mol), refluxed for 5 h. The reaction mixture was precipitated to obtain the tilted product; ¹H-NMR (300 MHz, CDCl₃): δ 8.02 (s, 2H), 7.52 (d, J=7.5 Hz, 2H), 7.13 (d, J=7.5 Hz, 4H), 4.73 (m, 2H), 2.87 (m, 8H).

### Example 64

to a mixture of paraformaldehyde 0.94 g (0.025 mole), acetic acid bromide 0.77 g (0.01 mole) and ethanol 5 ml was added 1,3-bis(4-(4-hydroxy-3-nitrophenoxy)phenyl)propan-2-one 2.58 g (0.005 mole), refluxed for 5 h. The reaction mixture was precipitated to obtain the title product; ¹H-NMR (300 MHz, CDCl₃): δ8.05 (d, J=7.5 Hz, 2H), 7.32 (d, J=7.5 Hz, 4H), 7.26 (d, J=7.5 Hz, 4H), 6.95 (s, 2H), 6.77 (d, J=7.5 Hz, 2H), 4.73 (m, 2H), 2.87 (m, 8H).

### Example 65

to a mixture of paraformaldehyde 0.94 g (0.025 mole), ammonium acetate 0.77 g (0.01 mole) and ethanol 5 ml was added 1,3-bis(4-nitro-3-hydroxyphenyl)propan-2-one 1.66 g (0.005 mole) and refluxed for 5 h. The reaction mixture was precipitate to obtain the objective product; ¹H-NMR (300 MHz, CDCl₃): δ8.02 (s, 2H), 7.52 (d, J=7.5 Hz, 2H), 7.13 (d, J=7.5 Hz, 4H), 4.73 (m, 2H), 2.87 (m, 8H).

### Example 66

to a mixture of ammonium acetate 129 g (1.67 mol) in ethanol 200 ml, nitromethane 33.3 g (0.544 mol) was added paraformaldehyde 111 g (3.70 mol), refluxed for 1 h. The resulting white crystals cold was washed with ethanol to obtain the title product; ¹H-NMR (300 MHz, DMSO-d6): δ 2.74-2.91 (br, 6H), 3.44 (m, 6H).

### Example 67

the title compound was synthesized by general method C with starting meterial 7-nitro-1,3,5-triaza adamantane 46.1 g (0.25 mol); IR: 2920, 1519, 1453, 1370, 1336, 1306, 1237, 1078, 997 ¹H-NMR (300 MHz, DMSO-d6): δ 5.11 (br, 2H), 3.44 (m, 6H), 2.67 (m, 3H), 2.42 (m, 3H).

### Example 68

the title compound was made by method C with starting meterial 7-amino-1,3,5-trinitroadamantane 30.8 g (0.2 mol) and amyl aldehyde 17.2 g (0.2 mol); IR: 3300; ¹H-NMR (300M Hz, CDCl₃): δ4.44, 4.08 (J=12 Hz, 6H), 3.29 (s, 6H), 2.56 (m, 2H), 1.33 (m, 7H), 0.90 (m, 3H).

### Example 69

to a mixture of acetic acid 1.38 g (5.53 mmol), DMAP 0.61 g (4.90 mmol), EDCI 1.92 g (10.00 mmol), HOBT 0.68 g (5.00 mmol) and 1,3,5-triazaadamantane-7-amine 0.785 g (5.10 mmol) in THF 25 ml was added 2-(5-nitro-1,3-dioxoisoindol-2-yl), stirred for 5 h. The solution was extracted with ethyl acetate to give the crude product for the next step; the title compound was synthesized by general method C with starting meterial of above product 1.20 g; IR: 3443, 3239, 2908, 1766, 1688, 1642, 1547, 1402, 1268; ¹H-NMR (300 MHz, DMSO-d6): δ 8.0 (b, 1H), 5.12 (d, J=7.8 Hz, 2H), 4.69 (d, J=1.8 Hz, 1H), 4.09 (dd, J=1.8 Hz, J=8.4 Hz, 2H), 3.43 (m, J=7.8 Hz, 6H), 3.01 (m, 1H), 2.89 (m, 1H), 2.67 (m, 1H), 2.54 (m, 4H), 2.42 (m, 1H), 1.92 (m, 1H), 1.67 (m, 1H).

### Example 70

the title compound was synthesized by general method C with starting 7-amino-1,3,5-triaza adamantane; ¹H-NMR (300 MHz, CDCl₃): δ 4.44, 4.06 (J=12 Hz, 6H), 3.44 (s, 6H), 2.60 (m, 4H), 1.49 (m, 6H), δ 4.44, 4.08 (J=12 Hz, 6H), 3.29 (s, 6H).

### Example 71

to a solution of perchloric acid 0.25 ml (70%) was added spiro[bicyclo[3.3.1]nonano-3,2-oxiranyl]-7-one 1.01 g (6.1 mmol) in water 25 ml, stirred for 3 h. The reaction solution was concentrated to give the title product; IR: 3350 (s), 3220, 2930, 2910, 2870, 1370, 1340, 1140, 1075, 1045; ¹H-NMR (300 MHz, CDCl₃): δ 1.38 (d, J=12.3 Hz, 2H), 1.72-1.81 (m, 8H), 2.36 (br s, 2H), 2.89 (br, 1H), 3.41 (s, 2H), 3.89 (br, 1H).

### Example 72

to a mixture of cerium trichloride heptahydrate 27.4 g (73.5 mmol) in THF 365 ml, methyl magnesium bromide 19.5 ml (58.5 mmol) was added spiro[bicyclo[3.3.1]nonane-3,7,9-trione 5.00 g (23.8 mmol) in THF, stirred for 2 h and The title compound was separated by column chromatography; ¹H-NMR (300 MHz, CDCl₃): δ 1.21 (s, 3H), 1.61 (b, d, J≈12.8 Hz, 2H), 1.66 (d, J=12.2 Hz, 2H), 1.83 (d, J=12.8 Hz, 2H), 2.07 (s, 2H), 2.13 (d, J=12.2 Hz, 2H), 3.82 (s, 1H), 3.96-3.99 (m, 4H);

### Example 73

to a solution of 2N HCl 145 ml was added ketaloxoadamantane 5.12 g, (22.7 mmol) in dioxane solution 500 ml, refluxed overnight. The reaction solution was concentrated under reduced pressure title product; ¹H-NMR (300M Hz, CDCl₃): δ 1.32 (s, 3H), 1.95 (d, J=13.2 Hz, 2H), 2.01 (d, J=13.2 Hz, 2H), 2.05 (d, J=12.4 Hz, 2H), 2.22 (d, J=12.4 Hz, 2H), 2.73 (b, 2H), 4.28 (s, 1H).

### Example 74

to a mixture of hydroxylamine HCl 5.04 g (72.5 mmol), sodium carbonate, 61.8 mmol, potassium carbonate 47.5 mmol was added adamantanone 2.58 g (14.2 mmol) in 1,4-dioxane 85 ml, refluxed 12 h. The filtrate was removed to give a white solid; IR: 3364, 1664; ¹H-NMR (300 MHz, CDCl₃): δ 1.19 (s, 3H), 1.64 (ddd, J=13.0 Hz, J=3.5 Hz, J=1.0 Hz, 1H), 1.67 (dd, J=13.0 Hz, J=3.5 Hz, J=1.5 Hz, 1H), 1.74 (ddd, J=13.0 Hz, J=J=3.0 Hz, 1H), 1.76-1.84 (m, 4H), 1.87 (dddd, J=12.0 Hz, J=J=3.0 Hz, J=1.0 Hz, 1H), 2.78 (m, 1H), 3.80 (m, 1H), 4.84 (s, 1H).

### Example 75

to a mixture of nickel dichloride hexahydrate 494 mg (2.08 mmol) in methanol 40 ml, sodium borohydride 236 mg (6.24 mmol) and sodium borohydride 552 mg (14.6 mmol) was added adamantane oxo oxime 820 mg (4.16 mmol) in methanol 10 ml, stirred for 1 h, The title compound was separated by column chromatography; IR: 3600-2400; ¹H-NMR (300 MHz, CDCl₃): δ2.61-2.92 (m, 1H), 1.64-1.72 (m, 2H), 1.50-1.64 (m, 3H), 1.30 (s, 3H), 1.10-1.35 (m, 5H).

### Example 76

to a solution of concentrated nitric acid 2.5 ml was added 2-oxo-adamantane 270 mg (2 mmole), stirred for 1.5 h, nitric acid was removed by evaporation under reduced pressure, water 1 ml and concentrated sulfuric acid (96%) 0.4 ml were added and was stirred for 1 h, 100° C. The title compound was separated by column chromatography; IR: 3220-35, 1075, 1020; ¹H-NMR (300 MHz, CDCl₃): δ3.32-3.68 (m, 2H), 1.06-1.85 (m, 11H).

### Example 77

to a mixture of acetic acid 4.2 ml and lead tetraacetate 1.3 g (2.9 mmol) was added 2-oxoadamantane 300 mg (2.2 mmol), refluxed for 20 h, The title compound was separated by column chromatography; IR: 1745, 1250, 1075, 1020; ¹H-NMR (300 MHz, CDCl₃): δ3.35-3.62 (m, 2H), 2.21 (s, 6H), 1.12-1.80 (m, 10H).

### Example 78

to a mixture of diacetoxy oxo-adamantane 130 mg in ethanol 10 ml was added 6-fold amount of 60% aqueous solution of potassium hydroxide, refluxed for 2 h to give the product; IR: 3200-3500, 1075, 1020; ¹H-NMR (300 MHz, CDCl₃): δ 4.40-5.22 (br, 2H), 3.32-3.64 (m, 2H), 1.12-1.80 (m, 10H).

### Example 79

to a mixture of bromine 3 ml and aluminum tribromide, 300 mg was added 2-oxo-adamantane 200 mg (1.5 mmol), stirred for 80 h, at 60° C., the reaction mixture was precipitate and the filtrate was purified by column chromatography to give the title product; IR: 1050, 1020; ¹H-NMR (300 MHz, CDCl₃): δ 6.02 (m, 1H), 3.32-3.64 (m, 2H), 1.10-1.70 (m, 10H).

### Example 80

to benzyl amine 4.29 g (40.0 mmol) was added bicyclo[3.3.1]nonane-3,7-dione 6.00 g (39.4 mmol) in THF 200 ml, refluxed for 30 min, LiAlH₄ (3.00 g, 79.0 mmol) in diethyl ether 80 ml was added, stirred for 6 h. and the precipitate was recrystallized to obtain the title product IR: 2927, 2712, 2408, 2377, 1569, 1323, 1206, 1194, 1126, 1093, 1008; ¹H-NMR (300 MHz, CDCL₃): δ1.78 (d, J=12.5 Hz, 2H), 1.90 (dquint, J=14.0 Hz, J=2.5 Hz, 1H), 1.97 (dtt, J=14.0 Hz, J=2.5 Hz, J00=1.5 Hz, 1H), 2.01-2.06 (m, 4H), 2.14 (d, J=11.5 Hz, 2H), 2.40 (b, 2H), 4.26 (s, 2H), 4.39 (b, 1H), 4.86 (1H), 7.42-7.49 (m, 3H), 7.50 (m, 2H).

### Example 81

to a solution of phenethylamine 2.55 g (21.1 mmol) was added to bicyclo[3.3.1]nonane-3,7-dione (3.00 g, 19.7 mmol) in THF 100 ml, refluxed for 30 min, and then LiAlH-43.00 g (79.0 mmol) in diethyl ether (80 ml) was added, stirred for 6 h. The solution was precipitated and purified to give title product; IR: 2934, 2855, 2721, 2674, 2617, 2419, 1604, 1467, 1455, 1324, 1209, 1192, 1093, 1018, 1001, 784, 725, 698; ¹H-NMR (300 MHz, CD₃OD): δ 1.74 (d, J=14.0 Hz, 2H), 1.87 (d, J=13.0 Hz, J=2.5 Hz, 1H), 1.95 (overlapped d, 1H), 1.96-2.03 (m, 4H), 2.06 (d, J=11.0 Hz, 2H), 2.38 (b, 2H), 2.99 (m, 2H), 3.28 (m, 2H), 4.33 (b, 1H), 4.86 (b, active-H), 7.27 (t, J=7.5 Hz, 1H), 7.29 (d, J=7.5 Hz, 2H), 7.35 (t, J=7.5 Hz, 2H).

### Example 82

to a mixture of acetonitrile 20 ml, formaldehyde solution 2.36 ml (30 mmol, 37%) and sodium cyanoborohydride 595 mg (9.00 mmol) was added 1-benzyl-2-oxoadamantane hydrochloride 838 mg (3.00 mmol), stirred for 30 min, glacial acetic acid 0.6 ml was added and the reaction mixture was concentrated. The title compound was purified by recrysterlization; IR: 2929, 2897, 2838, 1456, 1442, 1381, 1323, 1190, 994, 972, 957, 856, 747; ¹H-NMR (300 MHz, CD₃OD): δ 1.55 (d, J=13.5 Hz, 2H), 1.67 (b, J=12.0 Hz, 2H), 1.78 (d, J=13.5 Hz, 1H), 1.82 (d, J=13.5 Hz, 1H), 1.90 (d, J=13.5 Hz, 2H), 2.16 (d, J=12.0 Hz, 2H), 2.26 (b, 2H), 2.29 (s, 3H), 3.81 (s, 2H), 4.17 (br, 1H), 4.86 (br, 1H), 7.19 (t, J=7.5 Hz 1H), 7.28 (t, J=7.5 Hz, 2H), 7.32 (d, J=7.5 Hz, 2H).

### Example 83

to a mixture of formaldehyde 0.78 ml (10 mmol, 37%) and sodium cyanoborohydride 188 mg (3.00 mmol) was added 1-ethyl-2-oxoadamantane 257 mg (1.00 mmol) in acetonitrile 10 ml, stirred for 30 min and glacial acetic acid 0.3 ml was added. The solution was concentrated under reduced pressure to give a white title product; IR: 2956, 25961481, 1467, 1411, 1210; ¹H-NMR (300 MHz, CD₃OD): (free base) δ 1.54 (d, J=13.0 Hz, 2H), 1.59 (d, J=12.0 Hz, 2H), 1.74 (d, J=13.0 Hz, J=2.0 Hz, 2H), 1.80 (d, J=13.0 Hz, J=2.0 Hz, 2H), 1.88 (d, J=13.0 Hz, 2H), 2.07 (d, J=12.0 Hz, 2H), 2.23 (br, 2H), 2.47 (s, 3H), 2.79 (m, 2H), 2.89 (m, 2H), 4.14 (br, 1H), 4.86 (br, mobile H), 7.18 (t, J=7.5 Hz, 1H), 7.20 (d, J=7.5 Hz, 2H), 7.27 (t, J=7.5 Hz, 2H).

### Example 84

the title compound was synthesized by general method C with starting meterial, benzylmethyl oxoadamantyl: IR: 2928, 2856, 2750, 2694, 2416, 2372, 1467, 1209, 1157, 1097, 1078; ¹H-NMR (300 MHz, CD₃OD): δ 1.75 (d, J=12.5 Hz, 2H), 1.88 (d, J=13.0 Hz, J=2.5 Hz, 1H), 1.95 (m, 1H), 1.97 (m, 4H), 1.99 (m, 2H), 2.39 (b, 2H), 2.64 (s, 3H), 4.33 (b, 1H), 4.86 (br, active-H).

### Example 85

the title compound was synthesized by general method B with starting meterial, benzyl-2-oxo-adamantane hydrochloride; IR: 3034, 2945, 2851, 2789, 2744, 2697, 2631, 2563, 1578, 1502, 1384, 1359, 1329, 1304, 1211, 1156, 1016, 996; ¹H-NMR (300 MHz, CD₃OD): δ 1.74 (d, J=13.0 Hz, 2H), 1.86 (d, J=13.5 Hz, J=2.5 Hz, 1H), 1.95 (m, 1H), 1.96 (s, 4H), 1.98 (m, 2H), 2.35 (br, 2H), 4.28 (br, 1H), 4.86 (br, active-H).

### Example 86

to a mixture of potassium carbonate 690 mg (5.00 mmol), benzyl chloride 0.14 ml (1.25 mmol), sodium iodide 50 mg (0.33 mmol) and acetonitrile 10 ml was added benzyl-2-oxoadamantane hydrochloride 280 mg (1.00 mmol), refluxed for 18 h, The title compound was separated by column chromatography; IR: 2932, 2922, 2851, 1600, 1493, 1449, 1382, 1321, 1198, 1158, 1122, 986; ¹H-NMR (300 MHz, CD₃OD): δ 1.54 (d, J=12.5 Hz, 2H), 1.59 (d, J=12.0 Hz, 2H), 1.72 (d, J=12.5 Hz, 1H), 1.76 (br, J=12.5 Hz, 1H), 1.90 (d, J=12.5 Hz, 2H), 2.14 (d, J=12.0 Hz, 2H), 2.18 (br, 2H), 4.01 (s, 4H), 4.21 (br, 1H), 4.86 (br, active-H), 7.12 (t, J=7.5 Hz, 2H), 7.20 (t, J=7.5 Hz, 4H), 7.30 (d, J=7.5 Hz, 4H).

### Example 87

to a mixture of anhydrous hydrazine 68.5 ml (98%, 1.38 mol), concentrated hydrochloric acid 2.2 ml was added the hydroxyl-oxo-adamantane 10.5 g (62.5 mmol), refluxed for 18 h. The reaction mixture was precipitated and filtrated. Title compound was obtained by crystallization; IR: 3180, 2923, 2681, 1690, 1611, 1528, 1509, 1497, 1383, 1106, 1077, 943, 839; ¹H-NMR (300 MHz, CD₃OD): δ 1.16 (s, 3H), 1.60 (d, J=13.5 Hz, 2H), 1.63 (m, 2H), 1.66 (m, J=12.5 Hz, 2H),1.74 (d, J=12.5 Hz, 2H), 1.79 (m, 2H), 2.31 (m, 2H), 4.86 (s, active-H).

### Example 88

the title compound was synthesized by general method C with starting meterial, oxoadamantane hydrochloride; IR: 2966, 2924, 2852, 1582, 1516, 1379, 1235, 1060, 1038; ¹H-NMR (300 MHz, CD₃OD): δ 1.18 (s, 3H), 1.66 (d, J=14.0 Hz, 2H), 1.70 (d, J=14.0 Hz, 2H), 1.81 (m, 2H), 1.85 (d, J=11.5 Hz, 2H), 1.90 (dd, J=11.5 Hz, J=2.5 Hz, 2H), 2.38 (b, 2H), 4.86 (s, active-H).

### Example 89

to a mixture of formaldehyde 4.85 ml (37%, 61 mmol) and formic acid 3.8 ml (98 mmol was added methyl oxoamantadine 410 mg (2.45 mmol) in diethyl ether 8 ml, stirred for 10 h. at 80° C. The sodium hydroxide solution 5 ml, 5 N was added dropwise and the organic phase was concentrated under reduced pressure to obtain the title product; IR: 2963, 2856, 2556, 2458, 1488, 1450, 1410; ¹H-NMR (300 MHz, CD₃OD): δ 1.22 (s, 3H), 1.69 (m, 2H), 1.71 (m, 2H), 1.82 (m, 2H), 1.85 (d, J=11.0 Hz, 2H), 2.05 (dd, J=11.0 Hz, J=2.0 Hz, 2H), 2.46 (m, 2H), 2.83 (s, 6H).

### Example 90

to a mixture of sodium cyanoborohydride 200 mg (95%, 3.20 mmol), glacial acetic acid 0.6 ml and acetaldehyde 0.56 ml (9.6 mmol) was added oxo-adamantane hydrochloride (350 mg, 1.60 mmol) in methanol 20 ml, stirred for 16 h, The title compound was separated by column chromatography; IR: 2972, 2933, 2855, 2645, 2579, 2484, 1458, 1446, 1377, 1033, 1014, 975, 949; ¹H-NMR (300 MHz, CD₃OD): δ 0.93 (t, J=7.5 Hz, 3H), 1.38 (t, J=7.5 Hz, 6H), 1.53 (q, J=7.5 Hz, 2H), 1.64 (d, J=13.0 Hz, 2H), 1.75 (d, J=13.0 Hz, 2H), 1.82 (m, 1H), 1.85 (m, 1H), 1.94 (d, J=12.5 Hz, 2H), 2.09 (d, J=12.5 Hz, 2H), 2.47 (t, J=2.5 Hz, 2H), 3.06 (b, 2H), 3.59 (b, 2H).

### Example 91

to a mixture of sodium cyanoborohydride 393 mg (5.93 mmol, 95%), acetic acid 0.3 ml and benzaldehyde 0.42 ml, (4.12 mmol) was added oxoadamantane hydrochloride 400 mg (1.84 mmol) in methanol 10 ml, stirred for 16 h. The title compound was separated by column chromatography; IR: 2922, 2851, 2725, 2656, 2619, 2414, 1566, 1463, 1056, 1042, 1007, 988, 749, 690; ¹H-NMR (300 MHz, CD₃OD): δ 0.96 (t, J=7.5 Hz, 3H), 1.56 (q, J=7.5 Hz, 2H), 1.67 (d, J=12.5 Hz, 2H), 1.77 (d, J=12.5 Hz, 2H), 1.87 (b, 2H), 1.98 (d, J=11.5 Hz, 2H), 2.04 (d, J=11.5 Hz, 2H), 2.46 (m, 2H), 4.25 (s, 2H), 4.86 (s, active-H), 7.42-7.50 (m, 5H).

### Example 92

to a mixture of formaldehyde solution 0.23 ml (0.29 mmol, 37%) and sodium cyanoborohydride 55 mg (0.83 mmol 95%), glacial acetic acid 0.2 ml was added benzyl-oxoamantadine 90 mg (0.29 mmol) in acetonitrile 10 ml. The mixture was stirred for 16 h The title compound was separated by column chromatography; IR: 2969, 2921, 2853, 2472, 2353, 1458, 1033, 1024, 972, 938, 750, 702; ¹H-NMR (300 MHz, CD₃OD): δ 0.99 (t, J=7.5 Hz, 3H), 1.60 (q, J=7.5 Hz, 2H), 1.69 (d, J=12.5 Hz, 2H), 1.77-1.84 (b, 2H), 1.87 (m, 1H), 1.89 (m, 1H), 1.94-2.08 (b, 2H), 2.14-2.25 (b, 2H), 2.53 (b, 2H), 2.71 (s, 3H), 3.93 (b, 1H, J=8.0 Hz), 4.85 (m, 1H), 4.86 (s, active-H), 7.50 (m, 5H).

### Example 93

to a mixture of Pd/C 10 mg (10%) and ethanol 80 ml was added benzyl methyl oxoamantadine 390 mg (1.21 mmol) at 100° C. for 24 h. The mixture was filtered and the solvent was removed under reduced to obtain title compound; IR: 2968, 2931, 2848, 2706, 2592, 1561, 1474, 1118, 1068, 1057, 1028, 991, 972; ¹H-NMR (300 MHz, CD₃OD): δ 0.92 (t, J=7.5 Hz, 3H), 1.50 (q, J=7.5 Hz, 2H), 1.63 (d, J=12.5 Hz, 2H), 1.72 (d, J=12.5 Hz, 2H), 1.84 (s, 2H), 1.87 (d, J=13.0 Hz, 2H), 1.91 (d, J=13.0 Hz, 2H), 2.43 (br, 2H), 2.63 (s, 3H).

### Example 94

to a solution of hydrochloric acid 14 ml 0.5 was added N2-(3,5-bis[(tert-dimethylsilyl)oxy)]cyclohexyl)acetaldehyde, stirred for 2.5 h. The reaction solvent was evaporated under reduced pressure and title compound was obtained by silica gel column chromatography; IR: 3020, 2900; ¹H-NMR (300 MHz CDCl₃): δ1.40-2.70 (m, 9H), 4.19 (m, 2H), 5.11 (m, 1H).

### Example 95

to a mixture of inositol 10.80 g (60 mmol) and ethyl orthoformate 15 ml was added p-toluenesulfonic acid 1 g, 100° C., stirred for 1 h. To reaction mixture was added pyridine 60 ml and benzyl chloride 18.8 g (133 mmol), stirred for 18 h. The title compound was separated by column chromatography; IR: 3318, 2921, 2841, 1642, 1586, 1498, 1450, 1265, 1216, 1154.

### Example 96

to a mixture of DMAP (0.050 g), (1S)—(K)-camphanic chloride 0.566 g (2.614 mmol) and pyridine 10 ml was added racemic trioxoadamantane 1.000 g (2.008 mmol) at 80° C. for 10 h. The reaction mixture removed by evaporation under reduced pressure and the title product A and B were obtained by column chromatography; IR: 1788, 1768; ¹H-NMR (300 MHz, CDCl₃): δ 7.83 (d, J=10.0 Hz, 2H), 7.78 (d, J=10.0 Hz, 2H), 7.43 (d, J=10.0 Hz, 2H), 7.39 (d, J=10.0 Hz, 2H), 5.50 (s, 1H), 5.48-5.45 (m, 1H), 5.10-5.05 (m, 1H), 4.74 (d, J=2.0 Hz, 1H), 4.52-4.48 (m, 1H), 4.23-4.20 (m, 1H), 4.07-4.03 (m, 1H), 2.48 (s, 3H), 2.45 (s, 3H), 2.45-2.40 (m, 1H), 2.05-1.90 (m, 2H), 1.73-1.65 (m, 1H), 1.15 (s, 3H), 1.07 (s, 3H), 0.95 (s, 3H). Compound B, IR: 1776; ¹H-NMR (300 MHz, CDCl₃): δ 7.83 (d, J=10.0 Hz, 2H), 7.73 (d, J=10.0 Hz, 2H), 7.50-7.40 (m, 4H), 5.65-5.55 (m, 1H), 5.48 (d, J=5.0 Hz, 1H), 5.00-4.93 (m, 1H), 4.90-4.85 (m, 1H), 4.40-4.30 (m, 2H), 4.15-4.05 (m, 1H), 2.48 (s, 6H), 2.55-2.40 (m, 1H), 2.12-2.05 (m, 1H), 2.05-1.90 (m, 1H), 1.80-1.70 (m, 1H), 1.15 (s, 3H), 1.10 (s, 3H), 0.98 (s, 3H);

### Example 97

to a mixture of phloroglucinol 12.89 g (102.2 mmol) and ethanol 9 ml, 90% and sodium hydrogen carbonate 8.59 g (102.2 mmol) was added pyridoxal hydrochloride 20.81 g (102.2 mmol), refluxed 1 h. The title compound was separated by column chromatography for next step. Phloroglucinol pyridine 28.1 g (102.1 mmol) was suspended in M HCl 0.5 mol 100 ml), refluxed for 15 min, 170-180° C. The precipitated solid was filted and title compound was obtained by recrystlization; IR: 3350, 2900, 2825, 1590, 1520, 1430, 1395, 1270, 1210, 1110, 1065; ¹H-NMR (300 MHz, CDCl₃): δ 2.50 (s, 3H), 3.32 (s, 6H), 4.85 (m, 2H), 5.90 (m, 1H), 8.11 (s, 1H).

### Example 98

to a mixture of 4-(piperidin-1-yl)-1-p-toluenesulfonyl-1,2,3,6-tetrahydropyridine 1.53 g (5 mmol), triethylamine 0.606 g (6 mmol), and acetonitrile 20 ml was added 2-benzoyl-1,3-dichloropropane 1.085 g (5 mmol), stirred for 1 h. The solvent was removed by evaporation under reduced pressure and title compound was obtain; IR: 1725, 1685, 1360, 1170; ¹H-NMR (100 MHz, CDCl₃): δ7.73 (d, J=7.5 Hz, 2H), 7.43 (d, J=7.5 Hz, 2H), 7.50-7.98 (m, 5H), 3.30-3.45 (m, 5H), 2.34 (s, 3H), 1.72-2.45 (m, 6H).

### Example 99

to a mixture of lithium aluminum hydride 152 mg (4 mmol) and THF 20 ml was added 7-benzoyl-3-p-toluenesulfonyl-3-azabicyclo[3.3.1]nonan-9-one 795 mg (2 mmol), stirred for 4 h. at 50° C. The solution was filtered and concentrated under reduced pressure to obtain the title product; IR: 3500, 1360, 1160; ¹H-NMR (100 MHz, CDCl₃): δ 4.32 (J=8 Hz, 0.6H), 4.20 (J=7 Hz, 0.4H), 3.70 (b, 0.4H), 3.65 (4a, 1.2H), 3.45 (4a, 0.6H), 3.40 (4b, 0.8H).

### Example 100

to a mixture of concentrated hydrochloric acid 20 ml and glacial acetic acid 20 ml was added 7-(hydroxy(phenyl)methyl)-3-p-toluenesulfonyl-3-azabicyclo[3.3.1]nonan-9-ol 460 mg (1.14 mmol), refluxed for 5 h. The title compound was separated by column chromatography; IR: 3400, 1600; ¹H-NMR (100 MHz, CDCl₃): δ 4.10 (s, 80%, H-2 5a), 4.00 (t, 80%, H-6 5a), 3.30 (AB, H-9), 3.00 (AB, H-8); silver carbonate/Si diatomaceous earth (3 g, 5 mmol) was suspendedin 50 ml of xylene, adding the hydroxy compound 70 mg (0.3 mmol) was refluxed. The title compound was separated by column chromatography; IR (cm-1): 1700; 1H-NMR (100 MHz, CDCl3): δ7.45 (5H, C₆H₅), 4.30 (s, H-2), 3.50 (m, 3H); 2.90 (m, 1H).

### Example 101

to a solution of acetonitrile 200 ml and bromomethyl acrylate 15.4 g (0.08 mole) in ethanol 200 ml was added 4-(piperidin-1-yl)-1-p-toluenesulfonyl-1,2,3,6-tetrahydropyridine 24.5 g (0.08 mol), refluxed for 5 h. The title compound was separated by column chromatography; IR: 1710, 1720, 1160, 1340; ¹H-NMR (100 MHz, CDCl₃): δ7.74 (d, J=7.5 Hz, 2H), 7.40 (d, J=7.5 Hz, 2H), 4.13 (q, J=8 Hz, 2H), 3.45 (d, J=7 Hz, 4H), 2.63 (t, J=7 Hz, 1H), 2.34 (s, 3H), 1.95-2.48 (m, 6H), 1.29 (t, J=8 Hz, 3H).

### Example 102

to a solution of boron trifluoride diethyl ether 15 ml was added ethanol-3-tosyl-3-aza-bicyclo[3.3.1]nonano-9-one 58.4 g (0.16 mol) in ethanedithiol 20 ml and chloroform 200 ml, stirred for 1 h. The was removed by evaporation under reduced pressure to obtain the title compound; IR: 1715, 2900, 2970, 1160, 1350; ¹H-NMR (100 MHz, CDCl₃): δ7.74 (d, J=7.5 Hz, 2H), 7.40 (d, J=7.5 Hz, 2H), 4.13 (q, J=8 Hz, 2H), 4.04 (t, J=7.1 Hz, 4H), 3.58 (d, J=7 Hz, 4H), 2.27 (t, J=7 Hz, 1H), 2.34 (s, 3H), 1.65-2.35 (m, 6H), 1.29 (t, J=8 Hz, 3H).

### Example 103

to a mixture of Swiss mud N±22 ml, ethanol 200 ml was added ethanol-3-tosyl-3-aza-spiro[bicyclo[3.3.1]nonano-9,2′-[1,3]dithiole-yl]-7-acetate 2.207 g (5 mmol), refluxed for 18 h. The solvent was evaporated under reduced pressure to obtain the title product; IR: 1720, 2870, 2929, 1160, 1340; ¹H-NMR (100 MHz, CDCl₃): δ7.72 (d, J=7.5 Hz, 2H), 7.38 (d, J=7.5 Hz, 2H), 4.13 (q, J=8 Hz, 2H), 3.56 (d, J=7 Hz, 4H), 2.27 (t, J=7 Hz, 1H), 2.34 (s, 3H), 1.35-1.80 (m, 8H), 1.29 (t, J=8 Hz, 3H).

### Example 104

to a mixture of lithium aluminum hydride 2.21 g (68.4 mmol) in THF 20 ml was added 3-p-toluenesulfonyl-3-azabicyclo[3,3,1]nonane-7-carboxylate 12.01 g (34.2 mmol) in THF 100 ml, stirred for 3 h. The solvent was evaporated under reduced to obtain the title product; IR: 3600, 2880, 2950, 1160, 1340; ¹H-NMR (100 MHz, CDCl₃): δ7.74 (d, J=7.5 Hz, 2H), 7.40 (d, J=7.5 Hz, 2H), 3.46-3.58 (m, 6H), 1.52 (m, J=7 Hz, 1H), 2.34 (s, 3H), 1.24-2.67 (m, 8H).

### Example 105

to a mixture of zinc chloride 19.49 g (143.0 mmol) in dichloromethane 300 ml was add thioglycolate 53.25 g (699.6 mmol), refluxed for 24 h. The mixture was poured into ice water and title compound was precipitated; IR: 2966.4, 2910.2, 2846.9, 2713.3, 1433.6, 1363.3, 1342.2, 1089.1. ¹H-NMR (100 MHz, CDCl₃): δ 2.18 (s).

### Example 106

to a mixture of triphenylphosphine bromide cobalt 15 g, boron trifluoride ether solution 4 ml was added dicycloheptadiene 453 g (2.18 M) in benzene 600 ml, refluxed for 12 h. The solution was extracted with methylene chloride to give the compound A for use in the next step synthesis. The compound A 219.0 g (0.73 M) was dissolved in glacial acetic acid 800 ml (containing 8.7 ml of concentrated hydrochloric acid) and of platinum oxide 15 g and was hydrogenated, 70° C., for 3 h. the crude product was purified by distillation to gove product B; ¹H-NMR (100 MHz, CDCl₃): δ 3.68 (s, 12H), 3.54 (m, 2H), 2.67 (m, 2H), 1.41-1.75 (m, 10H).

### Example 107

to a mixture of aluminum bromide 28 g (0.1 M) and cyclohexane 100 ml was added the compound B 159 g (0.53 M), refluxed for 3 h and the crude was recrystallized to give title product; ¹H-NMR (100 MHz, CDCl₃): δ 3.68 (s, 6H), 2.26 (s, 6H), 2.22-2.58 (m, 6H), 1.35-1.59 (m, 7H).

TABLE 1
Embodiment 1-249

| Ex- | | | |
|---|---|---|---|
| am- | | | |
| ple | Chemical Structure | M. Weight | Structure name |
| 1 | 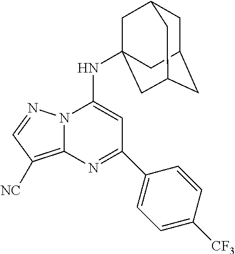 [SMILES: N#Cc1cnn2c(NC34CC5CC(CC(C5)C3)C4)cc(-c3ccc(C(F)(F)F)cc3)nc12] | 437.18 | 7-(adamantan-1-ylamino)-5-(4- (trifluoromethyl)phenyl)pyrazolo[1,5- a]pyrimidine-3-carbonitrile |
| 2 | 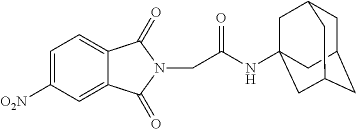 [SMILES: O=C(CN1C(=O)c2ccc([N+](=O)[O-])cc2C1=O)NC12CC3CC(CC(C3)C1)C2] | 383.15 | N-((3s,5s,7s)-adamantan-1-yl)-2-(5-nitro- 1,3-dioxoisoindolin-2-yl)acetamide |
| 3 | 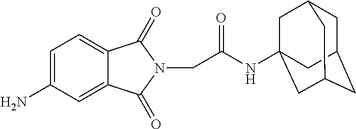 [SMILES: Nc1ccc2c(c1)C(=O)N(CC(=O)NC13CC4CC(CC(C4)C1)C3)C2=O] | 353.17 | N-((3s,5s,7s)-adamantan-1-yl)-2-(5-amino- 1,3-dioxoisoindolin-2-yl)acetamide |
| 4 | 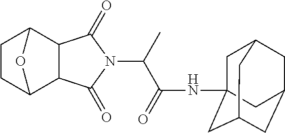 [SMILES: CC(C(=O)NC12CC3CC(CC(C3)C1)C2)N1C(=O)C2C3CCC(O3)C2C1=O] | 372.20 | N-((3s,5s,7s)-adamantan-1-yl)-2-(1,3- dioxohexahydro-1H-4,7-epoxyisoindol- 2(3H)-yl)propanamide |
| 5 | 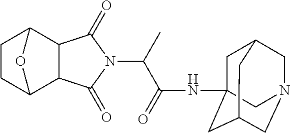 [SMILES: CC(C(=O)NC12CC3CC(CN(C3)C1)C2)N1C(=O)C2C3CCC(O3)C2C1=O] | 373.20 | N-((1r,3r,5R,7S)-1-azaadamantan-3-yl)- 2-(1,3-dioxohexahydro-1H-4,7- epoxyisoindol-2(3H)-yl)propanamide |
| 6 | 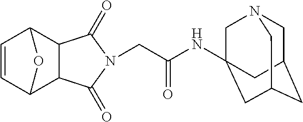 [SMILES: O=C(CN1C(=O)C2C3C=CC(O3)C2C1=O)NC12CC3CC(CN(C3)C1)C2] | 357.17 | N-((1s,3s)-1-azaadamantan-3-yl)-2-(1,3- dioxo-3a,4,7,7a-tetrahydro-1H-4,7- epoxyisoindol-2(3H)-yl)acetamid |
| 7 | 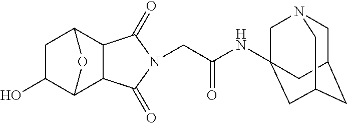 [SMILES: O=C(CN1C(=O)C2C3CC(O)C(O3)C2C1=O)NC12CC3CC(CN(C3)C1)C2] | 375.18 | N-((1r,3r)-1-azaadamantan-3-yl)-2- (5-hydroxy-1,3-dioxohexahydro-1H- 4,7-epoxyisoindol-2(3H)-yl)acetamide |
| 8 | 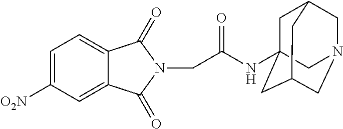 [SMILES: O=C(CN1C(=O)c2ccc([N+](=O)[O-])cc2C1=O)NC12CC3CC(CN(C3)C1)C2] | 384.14 | N-((1r,3r,5R,7S)-1-azaadamantan- 3-yl)-2-(5-nitro-1,3-dioxoisoindolin- 2-yl)acetamide |
| 9 | 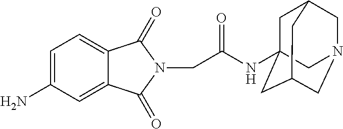 [SMILES: Nc1ccc2c(c1)C(=O)N(CC(=O)NC13CC4CC(CN(C4)C1)C3)C2=O] | 354.17 | N-((1r,3r,5R,7S)-1-azaadamantan- 3-yl)-2-(5-amino-1,3-dioxoisoindolin- 2-yl)acetamide |
| 10 | 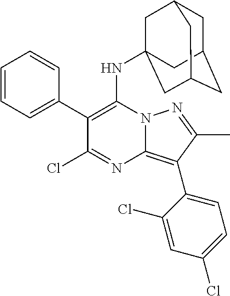 [SMILES: Cc1nn2c(NC34CC5CC(CC(C5)C3)C4)c(-c3ccccc3)c(Cl)nc2c1-c1ccc(Cl)cc1Cl] | 536.13 | (3S)-N-(adamantan-1-yl)-5-chloro-3- (2,4-dichlorophenyl)-2-methyl-6- phenylpyrazolo[1,5-a]pyrimidin-7- amine |
| 11 | 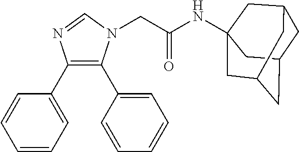 [SMILES: O=C(Cn1cnc(-c2ccccc2)c1-c1ccccc1)NC12CC3CC(CC(C3)C1)C2] | 411.23 | N-((1s,3s)-adamantan-1-yl)-2-(4,5- diphenyl-1H-imidazol-1-yl)acetamide |
| 12 | 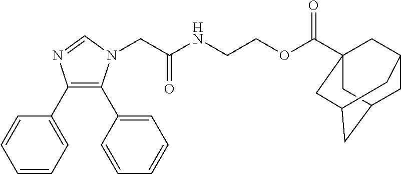 [SMILES: O=C(Cn1cnc(-c2ccccc2)c1-c1ccccc1)NCCOC(=O)C12CC3CC(CC(C3)C1)C2] | 483.25 | (1s,3s)-2-(2-(4,5-diphenyl-1H-imidazol- 1-yl)acetamido)ethyl adamantane-1- carboxylate |
| 13 | 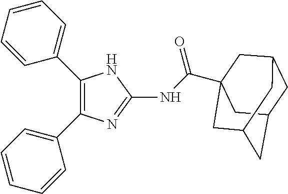 [SMILES: O=C(Nc1nc(-c2ccccc2)c(-c2ccccc2)[nH]1)C12CC3CC(CC(C3)C1)C2] | 397.22 | (1s,3s)-N-(4,5-diphenyl-1H-imidazol-2- yl)adamantane-1-carboxamide |
| 14 | 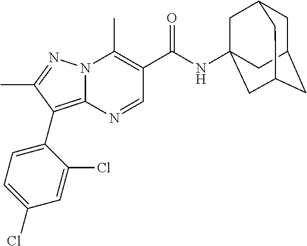 [SMILES: Cc1nn2c(C)c(C(=O)NC34CC5CC(CC(C5)C3)C4)cnc2c1-c1ccc(Cl)cc1Cl] | 468.15 | N-((3s,5s,7s)-adamantan-1-yl)-3-(2,4- dichlorophenyl)-2,7-dimethylpyrazolo[1,5- a]pyrimidine-6-carboxamide |
| 15 | 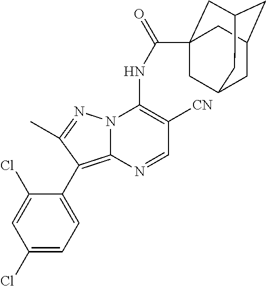 [SMILES: Cc1nn2c(NC(=O)C34CC5CC(CC(C5)C3)C4)c(C#N)cnc2c1-c1ccc(Cl)cc1Cl] | 479.13 | (3r,5r,7r)-N-(6-cyano-3-(2,4- dichlorophenyl)-2-methylpyrazolo [1,5-a]pyrimidin-7-yl)adamantane- 1-carboxamide |
| 16 | 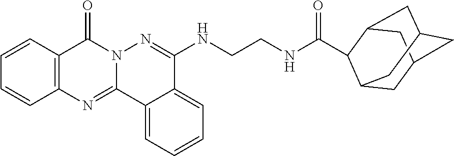 [SMILES: O=C(NCCNc1nn2c(=O)c3ccccc3nc2c2ccccc12)C1C2CC3CC(C2)CC1C3] | 467.23 | N-(2-((8-oxo-8H-phthalazino[1,2-b] quinazolin-5-yl)amino)ethyl)adamantane- 1-carboxamide |
| 17 | 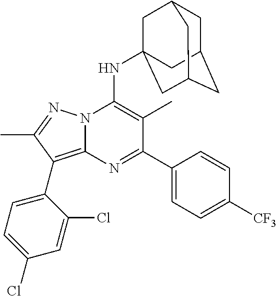 [SMILES: Cc1nn2c(NC34CC5CC(CC(C5)C3)C4)c(C)c(-c3ccc(C(F)(F)F)cc3)nc2c1-c1ccc(Cl)cc1Cl] | 584.17 | N-((3s,5s,7s)-adamantan-1-yl)-3-(2,4- dichlorophenyl)-2,6-dimethyl-5-(4- (trifluoromethyl)phenyl)pyrazolo[1,5- a]pyrimidin-7-amine |
| 18 | 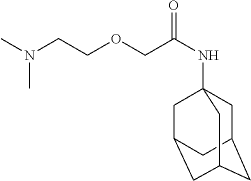 [SMILES: CN(C)CCOCC(=O)NC12CC3CC(CC(C3)C1)C2] | 280.22 | N-(adamantan-1-yl)-2-(2- (dimethylamino)ethoxy)acetamide |
| 19 | 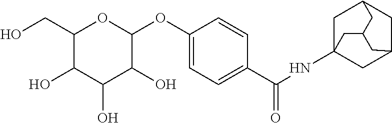 [SMILES: O=C(NC12CC3CC(CC(C3)C1)C2)c1ccc(OC2OC(CO)C(O)C(O)C2O)cc1] | 433.21 | N-(adamantan-1-yl)-4-((3,4,5-trihydroxy-6- (hydroxymethyl)tetrahydro-2H-pyran-2- yl)oxy)benzamide |
| 20 | 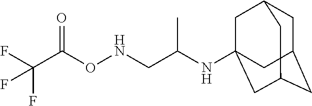 [SMILES: CC(CNOC(=O)C(F)(F)F)NC12CC3CC(CC(C3)C1)C2] | 320.17 | (3s,5s,7s)-N-(1-((2,2,2- trifluoroacetoxy)amino)propan-2- yl)adamantan-1-amine |
| 21 | 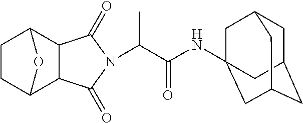 [SMILES: CC(C(=O)NC12CC3CC(CC(C3)C1)C2)N1C(=O)C2C3CCC(O3)C2C1=O] | 372.20 | N-(adamantan-1-yl)-2-(1,3- dioxohexahydro-1H-4,7- epoxyisoindol-2(3H)-yl)propanamide |
| 22 | 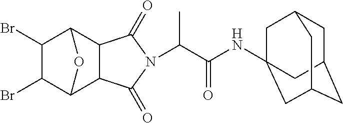 [SMILES: CC(C(=O)NC12CC3CC(CC(C3)C1)C2)N1C(=O)C2C3OC(C(Br)C3Br)C2C1=O] | 528.03 | N-((1S,3s)-adamantan-1-yl)-2- (5,6-dibromo-1,3-dioxohexahydro- 1H-4,7-epoxyisoindol-2(3H)- yl)propanamide |
| 23 | 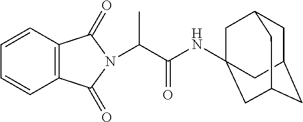 [SMILES: CC(C(=O)NC12CC3CC(CC(C3)C1)C2)N1C(=O)c2ccccc2C1=O] | 352.18 | N-(adamantan-1-yl)-2-(1,3- dioxoisoindolin-2-yl)propanamide |
| 24 | 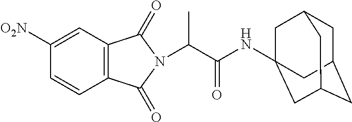 [SMILES: CC(C(=O)NC12CC3CC(CC(C3)C1)C2)N1C(=O)c2ccc([N+](=O)[O-])cc2C1=O] | 397.16 | N-(adamantan-1-yl)-2-(5-nitro-1,3- dioxoisoindolin-2-yl)propanamide |
| 25 | 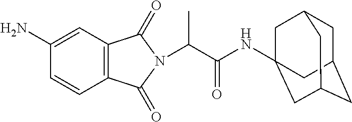 [SMILES: CC(C(=O)NC12CC3CC(CC(C3)C1)C2)N1C(=O)c2ccc(N)cc2C1=O] | 367.19 | N-(adamantan-1-yl)-2-(6-amino-1,3- dioxoisoindolin-2-yl)propanamide |
| 26 | 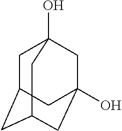 [SMILES: OC12CC3CC(C1)CC(O)(C3)C2] | 168.12 | (1s,3s,5s,7s)-adamantane-1,3-diol |
| 27 | 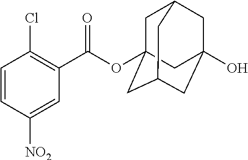 [SMILES: O=C(OC12CC3CC(CC(O)(C3)C1)C2)c1cc([N+](=O)[O-])ccc1Cl] | 351.09 | (1s,3r,5R,7S)-3-hydroxyadamantan- 1-yl 2-chloro-5-nitrobenzoate |
| 28 | 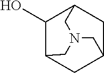 [SMILES: OC1C2CC3CC1CN(C3)C2] | 153.12 | 1-azaadamantan-4-ol |
| 29 | 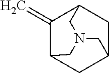 | 149.12 | 4-methylene-1-azaadamantane |
| 30 | 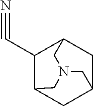 [SMILES: N#CC1C2CC3CC1CN(C3)C2] | 162.12 | 1-azaadamantane-4-carbonitrile |
| 31 | 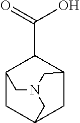 [SMILES: O=C(O)C1C2CC3CC1CN(C3)C2] | 181.11 | 1-azaadamantane-4-carboxylic acid |
| 32 | 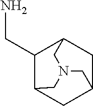 [SMILES: NCC1C2CC3CC1CN(C3)C2] | 166.15 | 1-azaadamantan-4-ylmethanamine |
| 33 | 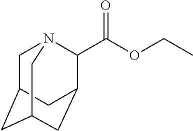 | 209.14 | (1r,3s,5R,7S)-ethyl 1-azaadamantane-2- carboxylate |
| 34 | 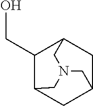 [SMILES: OCC1C2CC3CC1CN(C3)C2] | 167.13 | 1-azaadamantan-4-ylmethanol |
| 35 | 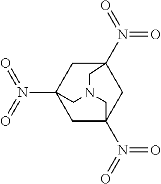 [SMILES: O=[N+]([O-])C12CN3CC([N+](=O)[O-])(C1)CC([N+](=O)[O-])(C3)C2] | 272.08 | 3,5,7-trinitro-1-azaadamantane |
| 36 | 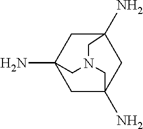 [SMILES: NC12CN3CC(N)(C1)CC(N)(C3)C2] | 182.15 | 1-azaadamantane-3,5,7-triamine |
| 37 | 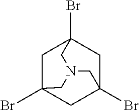 [SMILES: BrC12CN3CC(Br)(C1)CC(Br)(C3)C2] | 370.85 | 3,5,7-tribromo-1-azaadamantane |
| 38 | 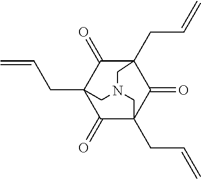 [SMILES: C=CCC12CN3CC(CC=C)(C1=O)C(=O)C(CC=C)(C3)C2=O] | 299.15 | 3,5,7-triallyl-1-azaadamantane-4,6,10-trione |
| 39 | 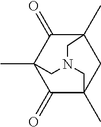 | 207.13 | 3,5,7-trimethyl-1-azaadamantane-4,6-dione |
| 40 | 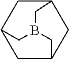 | 134.13 | 1-boraadamantane |
| 41 | 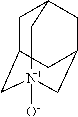 [SMILES: [O-][N+]12CC3CC(CC(C3)C1)C2] | 182.15 | 1-aza-adamantane-1-oxide |
| 42 | 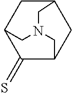 [SMILES: S=C1C2CC3CC1CN(C3)C2] | 167.08 | 1-azaadamantane-4-thione |
| 43 | 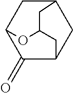 [SMILES: O=C1C2CC3CC(C2)OC1C3] | 152.08 | 2-oxaadamantan-4-one |
| 44 | 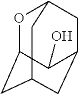 [SMILES: OC1C2CC3CC(C2)OC1C3] | 154.10 | (1S,3R,4S,5R,7S)-2-oxaadamantan-4-ol |
| 45 | 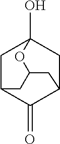 [SMILES: O=C1C2CC3CC1CC(O)(C2)O3] | 168.08 | 1-hydroxy-2-oxaadamantan-6-one |
| 46 | 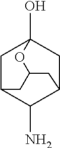 [SMILES: NC1C2CC3CC1CC(O)(C2)O3] | 169.11 | 6-amino-2-oxaadamantan-1-ol |
| 47 | 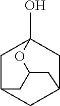 [SMILES: OC12CC3CC(CC(C3)O1)C2] | 154.10 | 2-oxaadamantan-1-ol |
| 48 | 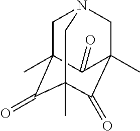 [SMILES: CC12CN3CC(C)(C1=O)C(=O)C(C)(C3)C2=O] | 221.11 | (3s,5s,7s)-3,5,7-trimethyl-1-azaadamantane- 4,6,10-trione |
| 49 | 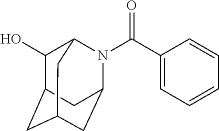 [SMILES: O=C(c1ccccc1)N1C2CC3CC(C2)C(O)C1C3] | 257.14 | ((1S,3R,4R,5R,7S)-4-hydroxy-2- azaadamantan-2-yl)(phenyl)methanone |
| 50 | 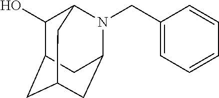 [SMILES: OC1C2CC3CC(C2)N(Cc2ccccc2)C1C3] | 243.16 | (1S,3R,4R,5R,7S)-2-benzyl-2- azaadamantan-4-ol |
| 51 | 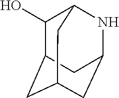 [SMILES: OC1C2CC3CC(C2)NC1C3] | 153.12 | (1S,3R,4R,5R,7S)-2-azaadamantan-4-ol |
| 52 | 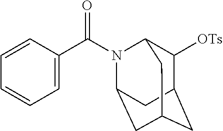 [SMILES: Cc1ccc(S(=O)(=O)OC2C3CC4CC(C3)N(C(=O)c3ccccc3)C2C4)cc1] | 411.15 | (1R,3S)-2-benzoyl-2-azaadamantan-4-yl 4- methylbenzenesulfonate |
| 53 | 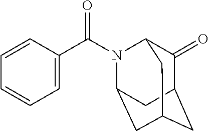 [SMILES: O=C1C2CC3CC(C2)N(C(=O)c2ccccc2)C1C3] | 255.13 | (1R,3S)-2-benzoyl-2-azaadamantan-4-one |
| 54 | 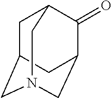 [SMILES: O=C1C2CC3CC1CN(C3)C2] | 151.10 | (1r,3R,5S,7s)-1-azaadamantan-4-one |
| 55 | 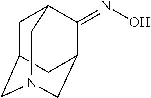 [SMILES: ON=C1C2CC3CC1CN(C3)C2] | 166.11 | (1R,3R,5S,7S,Z)-1-azaadamantan-4- one oxime |
| 56 | 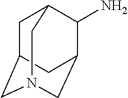 [SMILES: NC1C2CC3CC1CN(C3)C2] | 152.13 | (1r,3R,5S,7s)-1-azaadamantan-4-amine |
| 57 | 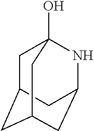 [SMILES: OC12CC3CC(CC(C3)N1)C2] | 153.12 | (1s,3s,5R,7S)-2-azaadamantan-1-ol |
| 58 | 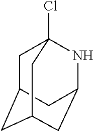 [SMILES: ClC12CC3CC(CC(C3)N1)C2] | 171.08 | (1s,3s,5R,7S)-1-chloro-2-azaadamantane |
| 59 | 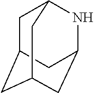 | 137.12 | (1r,3r,5r,7r)-2-azaadamantane |
| 60 | 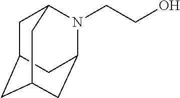 [SMILES: OCCN1C2CC3CC(C2)CC1C3] | 181.15 | 2-((1r,3r,5r,7r)-2-azaadamantan- 2-yl)ethanol |
| 61 | 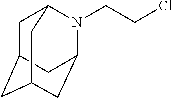 [SMILES: ClCCN1C2CC3CC(C2)CC1C3] | 199.11 | (1r,3r,5r,7r)-2-(2-chloroethyl)- 2-azaadamantane |
| 62 | 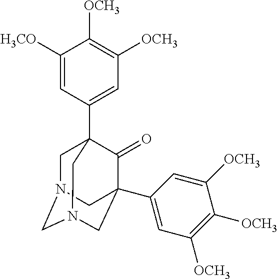 [SMILES: COc1cc(C23CN4CN(C2)CC(c2cc(OC)c(OC)c(OC)c2)(C4)C3=O)cc(OC)c1OC] | 484.22 | (1r,3r,5r,7r)-5,7-bis(3,4,5- trimethoxyphenyl)-1,3- diazaadamantan-6-one |
| 63 | 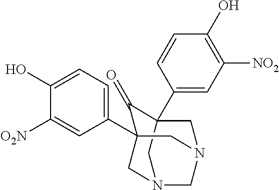 [SMILES: O=C1C2(c3ccc(O)c([N+](=O)[O-])c3)CN3CN(C2)CC1(c1ccc(O)c([N+](=O)[O-])c1)C3] | 426.12 | (1r,3r,5r,7r)-5,7-bis(4-hydroxy-3- nitrophenyl)-1,3-diazaadamantan- 6-one |
| 64 | 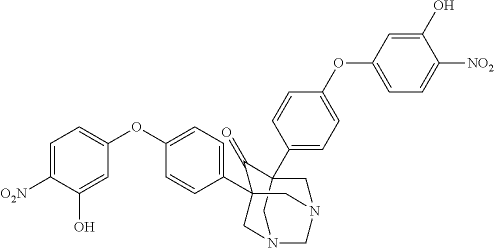 [SMILES: O=C1C2(c3ccc(Oc4ccc([N+](=O)[O-])c(O)c4)cc3)CN3CN(C2)CC1(c1ccc(Oc2ccc([N+](=O)[O-])c(O)c2)cc1)C3] | 610.17 | (1r,3r,5r,7r)-5,7-bis(4-(3-hydroxy-4- nitrophenoxy)phenyl)-1,3- diazaadamantan-6-one |
| 65 | 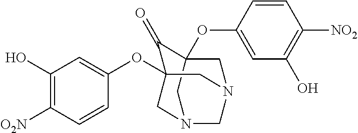 [SMILES: O=C1C2(Oc3ccc([N+](=O)[O-])c(O)c3)CN3CN(C2)CC1(Oc1ccc([N+](=O)[O-])c(O)c1)C3] | 458.11 | (1s,3s,5s,7s)-5,7-bis(3-hydroxy-4- nitrophenoxy)-1,3-diazaadamantan- 6-one |
| 66 | 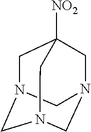 [SMILES: O=[N+]([O-])C12CN3CN(CN(C3)C1)C2] | 184.10 | (1s,3s,5s)-7-nitro-1,3,5-triazaadamantane |
| 67 | 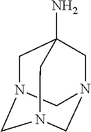 [SMILES: NC12CN3CN(CN(C3)C1)C2] | 154.12 | (1s,3s,5s)-1,3,5-triazaadamantan-7-amine |
| 68 | 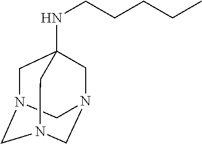 [SMILES: CCCCCNC12CN3CN(CN(C3)C1)C2] | 224.20 | (1s,3s,5s)-N-pentyl-1,3,5-triazaadamantan- 7-amine |
| 69 | 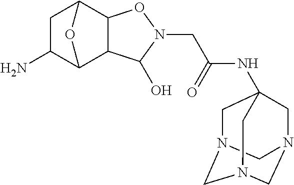 [SMILES: NC1CC2OC1C1C2ON(CC(=O)NC23CN4CN(CN(C4)C2)C3)C1O] | 366.20 | N-((1s,3s,5s)-1,3,5-triazaadamantan-7-yl)- 2-(5-amino-3-hydroxyhexahydro-4,7- epoxybenzo[d]isoxazol-2(3H)-yl)acetamide |
| 70 | 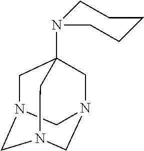 [SMILES: C1CCN(C23CN4CN(CN(C4)C2)C3)CC1] | 222.18 | (1s,3s,5s)-7-(piperidin-1-yl)-1,3,5- triazaadamantane |
| 71 | 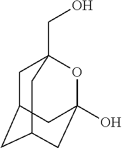 [SMILES: OCC12CC3CC(CC(O)(C3)O1)C2] | 184.11 | (1r,3s,5R,7S)-3-(hydroxymethyl)-2- oxaadamantan-1-ol |
| 72 | 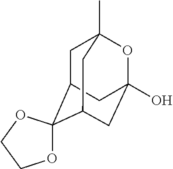 [SMILES: CC12CC3CC(O)(CC(C1)C31OCCO1)O2] | 226.12 | (1r,3s,5R,7S)-3-methyl-2-oxaspiro [adamantane-6,2′-[1,3]dioxolan]-1-ol |
| 73 | 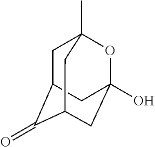 | 182.09 | (1r,3s,5R,7S)-1-hydroxy-3-methyl-2- oxaadamantan-6-one |
| 74 | 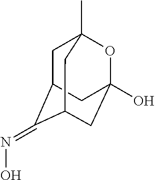 | 197.11 | (1R,3S,5R,7S,Z)-1-hydroxy-3-methyl-2- oxaadamantan-6-one oxime |
| 75 | 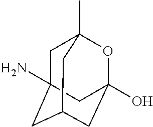 | 183.13 | (1S,3R,5R,7R)-5-amino-3-methyl-2- oxaadamantan-1-ol |
| 76 | 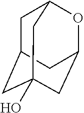 [SMILES: OC12CC3CC(C1)OC(C3)C2] | 154.10 | (1R,3S,5s,7s)-2-oxaadamantan-5-ol |
| 77 | 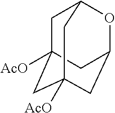 [SMILES: CC(=O)OC12CC3CC(OC(C)=O)(CC(C1)O3)C2] | 254.12 | (1r,3r,5s,7s)-2-oxaadamantane-5,7-diyl diacetate |
| 78 | 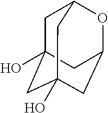 [SMILES: OC12CC3CC(O)(CC(C1)O3)C2] | 170.09 | (1r,3r,5s,7s)-2-oxaadamantane-5,7-diol |
| 79 | 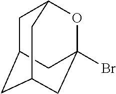 [SMILES: BrC12CC3CC(CC(C3)O1)C2] | 216.01 | (1s,3s,5R,7S)-1-bromo-2-oxaadamantane |
| 80 | 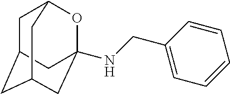 [SMILES: c1ccc(CNC23CC4CC(CC(C4)O2)C3)cc1] | 243.16 | (1r,3s,5R,7S)-N-benzyl-2-oxaadamantan-1- amine |
| 81 | 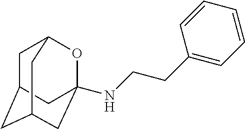 [SMILES: c1ccc(CCNC23CC4CC(CC(C4)O2)C3)cc1] | 257.18 | (1r,3s,5R,7S)-N-phenethyl-2-oxaadamantan- 1-amine |
| 82 | 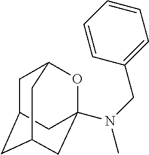 [SMILES: CN(Cc1ccccc1)C12CC3CC(CC(C3)O1)C2] | 257.18 | (1r,3s,5R,7S)-N-benzyl-N-methyl-2- oxaadamantan-1-amine |
| 83 | 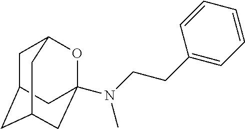 [SMILES: CN(CCc1ccccc1)C12CC3CC(CC(C3)O1)C2] | 271.19 | (1r,3s,5R,7S)-N-methyl-N-phenethyl-2- oxaadamantan-1-amine |
| 84 | 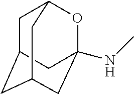 | 167.13 | (1r,3s,5R,7S)-N-methyl-2-oxaadamantan-1- amine |
| 85 | 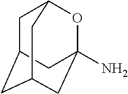 [SMILES: NC12CC3CC(CC(C3)O1)C2] | 153.12 | (1r,3s,5R,7S)-2-oxaadamantan-1-amine |
| 86 | 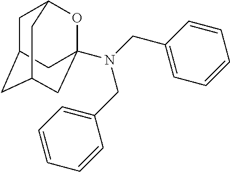 [SMILES: c1ccc(CN(Cc2ccccc2)C23CC4CC(CC(C4)O2)C3)cc1] | 333.21 | (1r,3s,5R,7S)-N,N-dibenzyl-2-oxaadamantan-1- amine |
| 87 | 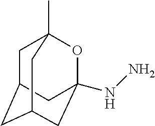 | 182.14 | ((1r,3s,5R,7S)-3-methyl-2-oxaadamantan-1- yl)hydrazine |
| 88 | 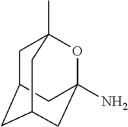 | 167.13 | (1r,3s,5R,7S)-3-methyl-2-oxaadamantan-1- amine |
| 89 | 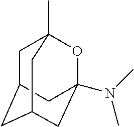 | 195.16 | (1r,3s,5R,7S)-N,N,3-trimethyl-2- oxaadamantan-1-amine |
| 90 | 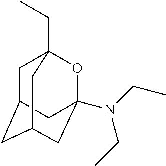 [SMILES: CCN(CC)C12CC3CC(CC(CC)(C3)O1)C2] | 237.21 | (1r,3s,5R,7S)-N,N,3-triethyl-2-oxaadamantan-1- amine |
| 91 | 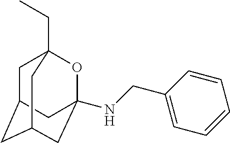 [SMILES: CCC12CC3CC(C1)CC(NCc1ccccc1)(C3)O2] | 271.19 | (1r,3s,5R,7S)-N-benzyl-3-ethyl-2- oxaadamantan-1-amine |
| 92 | 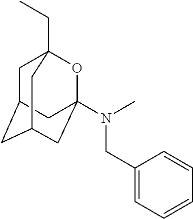 [SMILES: CCC12CC3CC(C1)CC(N(C)Cc1ccccc1)(C3)O2] | 285.21 | (1r,3s,5R,7S)-N-benzyl-3-ethyl-N-methyl-2- oxaadamantan-1-amine |
| 93 | 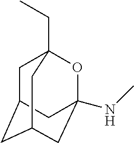 | 195.16 | (1r,3s,5R,7S)-3-ethyl-N-methyl-2- oxaadamantan-1-amine |
| 94 | 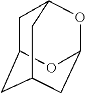 | 140.08 | (1R,3s,5S,7r)-2,4-dioxaadamantane |
| 95 | 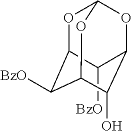 | 398.10 | (1R,3R,5S,6R,7S,8R,9S)-9-hydroxy-2,4,10- trioxaadamantane-6,8-diyl dibenzoate |
| 96 | 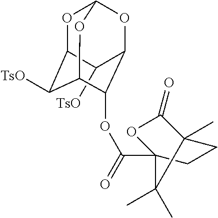 [SMILES: Cc1ccc(S(=O)(=O)OC2C3OC4OC(C3OC(=O)C35CCC(C)(C(=O)O3)C5(C)C)C(OS(=O)(=O)c3ccc(C)cc3)C2O4)cc1] | 678.14 | (1S,4S)-(1S,3R,5R,6S,7S,8R,9R)-8,9- bis(tosyloxy)-2,4,10-trioxaadamantan- 6-yl 4,7,7-trimethyl-3-oxo-2-oxabicyclo [2.2.1]heptane-1-carboxylate |
| | 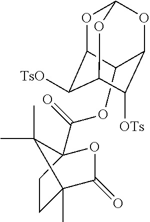 [SMILES: Cc1ccc(S(=O)(=O)OC2C3OC4OC(C3OC(=O)C35CCC(C)(C(=O)O3)C5(C)C)C(OS(=O)(=O)c3ccc(C)cc3)C2O4)cc1] | 678.14 | (1S,4S)-(1R,3S,5R,6R,7S,8S,9S)-8,9- bis(tosyloxy)-2,4,10-trioxaadamantan- 6-yl 4,7,7-trimethyl-3-oxo-2-oxabicyclo [2.2.1]heptane-1-carboxylate |
| 97.1 | 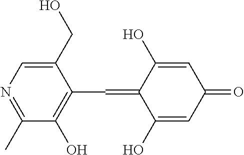 [SMILES: Cc1ncc(CO)c(C=C2C(O)=CC(=O)C=C2O)c1O] | 275.08 | 3,5-dihydroxy-4-((3-hydroxy-5- (hydroxymethyl)-2-methylpyridin-4- yl)methylene)cyclohexa-2,5-dienone |
| 97 | 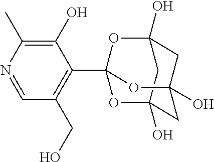 [SMILES: Cc1ncc(CO)c(C23OC4(O)CC(O)(CC(O)(C4)O2)O3)c1O] | 327.10 | 3-(3-hydroxy-5-(hydroxymethyl)-2- methylpyridin-4-yl)-2,4,10-trioxaadamantane- 1,5,7-triol |
| 98 | 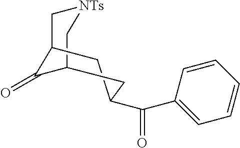 [SMILES: Cc1ccc(S(=O)(=O)N2CC3CC(C(=O)c4ccccc4)CC(C2)C3=O)cc1] | 397.13 | 7-benzoyl-3-tosyl-3-azabicyclo[3.3.1]nonan- 9-one |
| 99 | 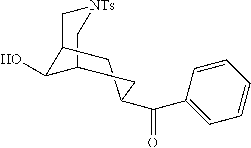 [SMILES: Cc1ccc(S(=O)(=O)N2CC3CC(C(=O)c4ccccc4)CC(C2)C3O)cc1] | 401.52 | 7-(hydroxy(phenyl)methyl)-3-tosyl-3- azabicyclo[3.3.1]nonan-9-ol |
| 100 | 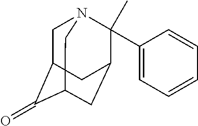 [SMILES: CC1(c2ccccc2)C2CC3CN1CC(C2)C3=O] | 241.33 | (1r,3R,5S,7s)-8-methyl-8-phenyl-1- azaadamantan-4-one |
| 101 | 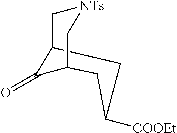 [SMILES: CCOC(=O)C1CC2CN(S(=O)(=O)c3ccc(C)cc3)CC(C1)C2=O] | 365.13 | (1R,5S,7s)-ethyl 9-oxo-3-tosyl-3- azabicyclo[3.3.1]nonane-7-carboxylate |
| 102 | 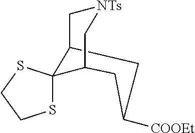 [SMILES: CCOC(=O)C1CC2CN(S(=O)(=O)c3ccc(C)cc3)CC(C1)C21SCCS1] | 441.11 | (1R,5S,7s)-ethyl 3-tosyl-3- azaspiro[bicyclo[3.3.1]nonane-9,2′- [1,3]dithiolane]-7-carboxylate |
| 103 | 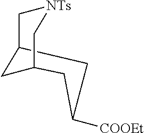 [SMILES: CCOC(=O)C1CC2CC(C1)CN(S(=O)(=O)c1ccc(C)cc1)C2] | 351.15 | (1R,5S,7s)-ethyl 3-tosyl-3- azabicyclo[3.3.1]nonane-7-carboxylate |
| 104 | 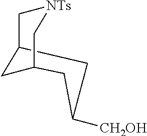 [SMILES: Cc1ccc(S(=O)(=O)N2CC3CC(CO)CC(C3)C2)cc1] | 309.14 | ((1R,5S,7s)-3-tosyl-3-azabicyclo [3.3.1]nonan-7-yl)methanol |
| 105 | 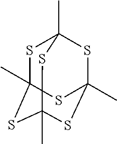 | 299.93 | 1,3,5,7-tetramethyl-2,4,6,8,9,10- hexathiaadamantane |
| 106 | 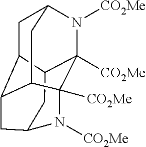 [SMILES: COC(=O)N1C2CC3C4CC5CC3C1(C(=O)OC)C(C(=O)OC)(C4C2)N5C(=O)OC] | 437.19 | Tetramethanol decahydro-2,8,4,6- (cyclobutyl[1,2,3,4]dimethyl) naphthalene- 2,4,4a,6-tetracarboxylate |
| 107 | 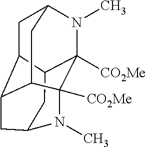 [SMILES: COC(=O)C12C3CC4CC5C3CC(CC5C1(C(=O)OC)N4C)N2C] | 334.19 | dimethanol decahydro-2,8,4,6- (cyclobutyl[1,2,3,4]dimethyl) naphthalene- 2,4,4a,6-dicarboxylate |
| 108 | 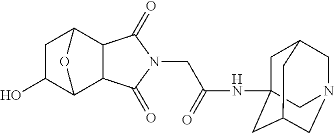 [SMILES: O=C(CN1C(=O)C2C3CC(O)C(O3)C2C1=O)NC12CC3CC(CN(C3)C1)C2] | 375.18 | N-((1r,3r,5R,7S)-1-azaadamantan-3-yl)-2-(5- hydroxy-1,3-dioxohexahydro-1H-4,7- epoxyisoindol-2(3H)-yl)acetamide |
| 109 | 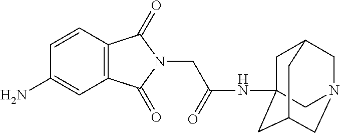 [SMILES: Nc1ccc2c(c1)C(=O)N(CC(=O)NC13CC4CC(CN(C4)C1)C3)C2=O] | 354.17 | N-((1r,3r,5R,7S)-1-azaadamantan-3-yl)-2-(5- amino-1,3-dioxoisoindolin-2-yl)acetamide |
| 110 | 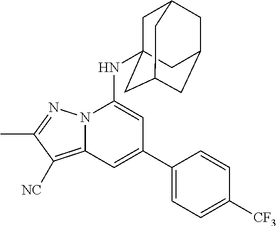 [SMILES: Cc1nn2c(NC34CC5CC(CC(C5)C3)C4)cc(-c3ccc(C(F)(F)F)cc3)cc2c1C#N] | 450.20 | 7-((3s,5s,7s)-adamantan-1-ylamino)-2- methyl-5-(4-(trifluoromethyl)phenyl)pyrazolo [1,5-a]pyridine-3-carbonitrile |
| 111 | 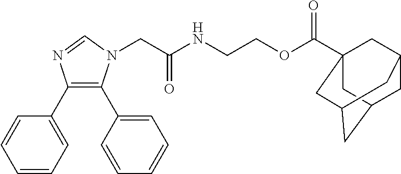 [SMILES: O=C(Cn1cnc(-c2ccccc2)c1-c1ccccc1)NCCOC(=O)C12CC3CC(CC(C3)C1)C2] | 483.25 | (1s,3s)-2-(2-(4,5-diphenyl-1H-imidazol-1- yl)acetamido)ethyl adamantane-1-carboxylate |
| 112 | 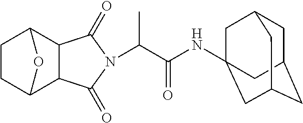 [SMILES: CC(C(=O)NC12CC3CC(CC(C3)C1)C2)N1C(=O)C2C3CCC(O3)C2C1=O] | 372.20 | N-(adamantan-1-yl)-2-(1,3-dioxohexahydro- 1H-4,7-epoxyisoindol-2(3H)-yl)propanamide |
| 113 | 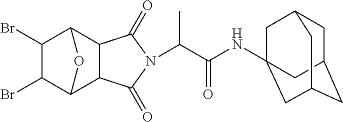 [SMILES: CC(C(=O)NC12CC3CC(CC(C3)C1)C2)N1C(=O)C2C3OC(C(Br)C3Br)C2C1=O] | 528.03 | N-(adamantan-1-yl)-2-(5,6-dibromo-1,3- dioxohexahydro-1H-4,7-epoxyisoindol-2(3H)- yl)propanamide |
| 114 | 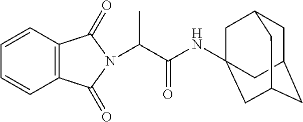 [SMILES: CC(C(=O)NC12CC3CC(CC(C3)C1)C2)N1C(=O)c2ccccc2C1=O] | 352.18 | N-(adamantan-1-yl)-2-(1,3-dioxoisoindolin-2- yl)propanamide |
| 115 | 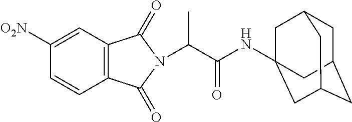 [SMILES: CC(C(=O)NC12CC3CC(CC(C3)C1)C2)N1C(=O)c2ccc([N+](=O)[O-])cc2C1=O] | 397.16 | N-(adamantan-1-yl)-2-(6-nitro-1,3- dioxoisoindolin-2-yl)propanamide |
| 116 | 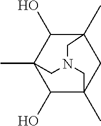 | 211.16 | 3,5,7-trimethyl-1-azaadamantane-4,6-diol |
| 117 | 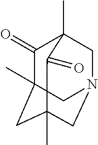 | 207.13 | (1s,3R,5r,7S)-3,5,7-trimethyl-1-azaadamantane- 4,6-dione |
| 118 | 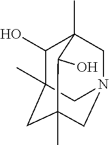 | 211.16 | (1S,3R,4R,5R,6R,7S)-3,5,7-trimethyl-1- azaadamantane-4,6-diol |
| 119 | 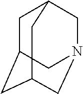 | 137.12 | (3s,5s,7s)-1-azaadamantane |
| 120 | 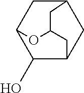 [SMILES: OC1C2CC3CC(C2)OC1C3] | 154.10 | 2-oxaadamantan-4-ol |
| 121 | 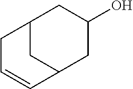 | 138.10 | bicyclo[3.3.1]non-6-en-3-ol |
| 122 | 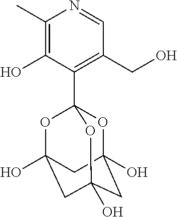 [SMILES: Cc1ncc(CO)c(C23OC4(O)CC(O)(CC(O)(C4)O2)O3)c1O] | 327.10 | (1s,5s,7s)-3-(3-hydroxy-5-(hydroxymethyl)-2- methylpyridin-4-yl)-2,4,10-trioxaadamantane- 1,5,7-triol |
| 123 | 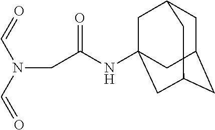 [SMILES: O=CN(C=O)CC(=O)NC12CC3CC(CC(C3)C1)C2] | 264.15 | N-((3s,5s,7s)-adamantan-1-yl)-2-(N- formylformamido)acetamide |
| 124 | 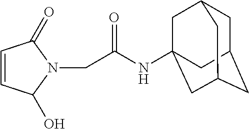 [SMILES: O=C(CN1C(=O)C=CC1O)NC12CC3CC(CC(C3)C1)C2] | 290.16 | N-((3s,5s,7s)-adamantan-1-yl)-2-(2-hydroxy-5- oxo-2,5-dihydro-1H-pyrrol-1-yl)acetamide |
| 125 | 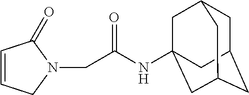 [SMILES: O=C(CN1CC=CC1=O)NC12CC3CC(CC(C3)C1)C2] | 274.17 | N-((3s,5s,7s)-adamantan-1-yl)-2-(2-oxo-2,5- dihydro-1H-pyrrol-1-yl)acetamide |
| 126 | 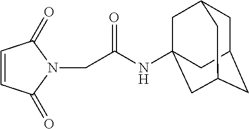 [SMILES: O=C(CN1C(=O)C=CC1=O)NC12CC3CC(CC(C3)C1)C2] | 290.16 | N-((3s,5s,7s)-adamantan-1-yl)-2-(2,5- dioxopyrrolidin-1-yl)acetamide |
| 127 | 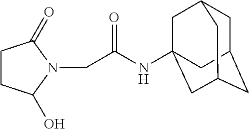 [SMILES: O=C(CN1C(=O)CCC1O)NC12CC3CC(CC(C3)C1)C2] | 292.18 | N-((3s,5s,7s)-adamantan-1-yl)-2-(2-hydroxy-5- oxopyrrolidin-1-yl)acetamide |
| 128 | 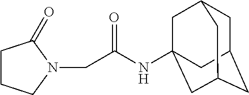 [SMILES: O=C(CN1CCCC1=O)NC12CC3CC(CC(C3)C1)C2] | 276.18 | N-((3s,5s,7s)-adamantan-1-yl)-2-(2- oxopyrrolidin-1-yl)acetamide |
| 129 | 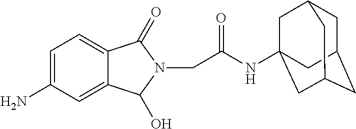 [SMILES: Nc1ccc2c(c1)C(O)N(CC(=O)NC13CC4CC(CC(C4)C1)C3)C2=O] | 355.19 | N-((3s,5s,7s)-adamantan-1-yl)-2-(5-amino-3- hydroxy-1-oxoisoindolin-2-yl)acetamide |
| 130 | 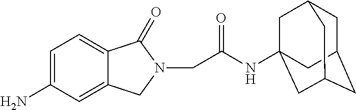 [SMILES: Nc1ccc2c(c1)CN(CC(=O)NC13CC4CC(CC(C4)C1)C3)C2=O] | 339.19 | N-((3s,5s,7s)-adamantan-1-yl)-2-(5-amino-1- oxoisoindolin-2-yl)acetamide |
| 131 | 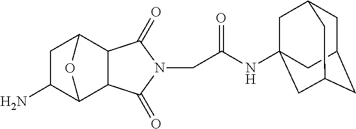 [SMILES: NC1CC2OC1C1C(=O)N(CC(=O)NC34CC5CC(CC(C5)C3)C4)C(=O)C21] | 373.20 | N-((3s,5s,7s)-adamantan-1-yl)-2-(5-amino-1,3- dioxohexahydro-1H-4,7-epoxyisoindol-2(3H)- yl)acetamide |
| 132 | 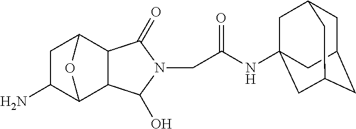 [SMILES: NC1CC2OC1C1C2C(=O)N(CC(=O)NC23CC4CC(CC(C4)C2)C3)C1O] | 375.22 | N-((3s,5s,7s)-adamantan-1-yl)-2-(5-amino-3- hydroxy-1-oxohexahydro-1H-4,7- epoxyisoindol-2(3H)-yl)acetamide |
| 133 | 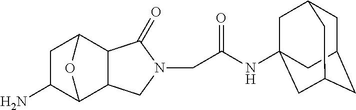 [SMILES: NC1CC2OC1C1CN(CC(=O)NC34CC5CC(CC(C5)C3)C4)C(=O)C21] | 359.22 | N-((3s,5s,7s)-adamantan-1-yl)-2-(5-amino-1- oxohexahydro-1H-4,7-epoxyisoindol-2(3H)- yl)acetamide |
| 134 | 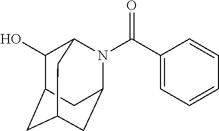 [SMILES: O=C(c1ccccc1)N1C2CC3CC(C2)C(O)C1C3] | 257.14 | ((1S,3R,4R,5R,7S)-4-hydroxy-2- azaadamantan-2-yl)(phenyl)methanone |
| 135 | 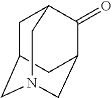 [SMILES: O=C1C2CC3CC1CN(C3)C2] | 151.10 | (1r,3R,5S,7s)-1-azaadamantan-4-one |
| 136 | 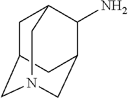 [SMILES: NC1C2CC3CC1CN(C3)C2] | 152.13 | (1r,3R,5S,7s)-1-azaadamantan-4-amine |
| 137 | 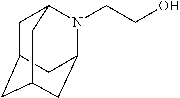 [SMILES: OCCN1C2CC3CC(C2)CC1C3] | 181.15 | 2-((1r,3r,5r,7r)-2-azaadamantan-2-yl)ethanol |
| 138 | 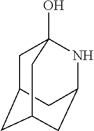 [SMILES: OC12CC3CC(CC(C3)N1)C2] | 153.12 | (1s,3s,5R,7S)-2-azaadamantan-1-ol |
| 139 | 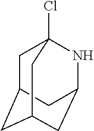 [SMILES: ClC12CC3CC(CC(C3)N1)C2] | 171.08 | (1s,3s,5R,7S)-1-chloro-2-azaadamantane |
| 140 | 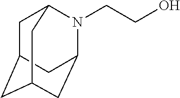 [SMILES: OCCN1C2CC3CC(C2)CC1C3] | 181.15 | 2-((1r,3r,5r,7r)-2-azaadamantan-2-yl)ethanol |
| 141 | 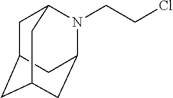 [SMILES: ClCCN1C2CC3CC(C2)CC1C3] | 199.11 | (1r,3r,5r,7r)-2-(2-chloroethyl)-2- azaadamantane |
| 142 | 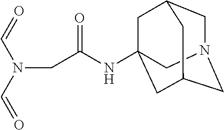 [SMILES: O=CN(C=O)CC(=O)NC12CC3CC(CN(C3)C1)C2] | 265.14 | N-((1r,3r,5R,7S)-1-azaadamantan-3-yl)-2-(N- formylformamido)acetamide |
| 143 | 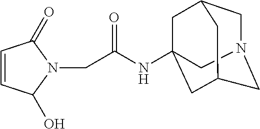 [SMILES: O=C(CN1C(=O)C=CC1O)NC12CC3CC(CN(C3)C1)C2] | 291.16 | N-((1r,3r,5R,7S)-1-azaadamantan-3-yl)-2-(2- hydroxy-5-oxo-2,5-dihydro-1H-pyrrol-1- yl)acetamide |
| 144 | 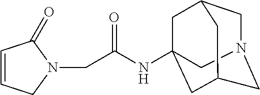 [SMILES: O=C(CN1CC=CC1=O)NC12CC3CC(CN(C3)C1)C2] | 275.16 | N-((1r,3r,5R,7S)-1-azaadamantan-3-yl)-2-(2- oxo-2,5-dihydro-1H-pyrrol-1-yl)acetamide |
| 145 | 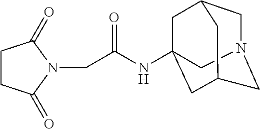 [SMILES: O=C(CN1C(=O)CCC1=O)NC12CC3CC(CN(C3)C1)C2] | 291.16 | N-((1r,3r,5R,7S)-1-azaadamantan-3-yl)-2-(2,5- dioxopyrrolidin-1-yl)acetamide |
| 146 | 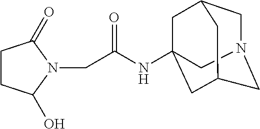 [SMILES: O=C(CN1C(=O)CCC1O)NC12CC3CC(CN(C3)C1)C2] | 293.17 | N-((1r,3r,5R,7S)-1-azaadamantan-3-yl)-2-(2- hydroxy-5-oxopyrrolidin-1-yl)acetamide |
| 147 | 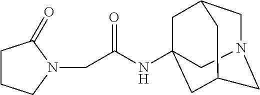 [SMILES: O=C(CN1CCCC1=O)NC12CC3CC(CN(C3)C1)C2] | 277.18 | N-((1r,3r,5R,7S)-1-azaadamantan-3-yl)-2-(2- oxopyrrolidin-1-yl)acetamide |
| 148 | 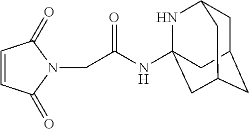 [SMILES: O=C(CN1C(=O)C=CC1=O)NC12CC3CC(CC(C3)N1)C2] | 289.14 | N-((1s,3s,5R,7S)-2-azaadamantan-1-yl)-2-(2,5- dioxo-2,5-dihydro-1H-pyrrol-1-yl)acetamide |
| 149 | 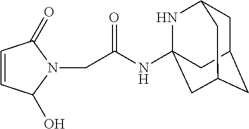 [SMILES: O=C(CN1C(=O)C=CC1O)NC12CC3CC(CC(C3)N1)C2] | 291.16 | N-((1s,3s,5R,7S)-2-azaadamantan-1-yl)-2-(2- hydroxy-5-oxo-2,5-dihydro-1H-pyrrol-1- yl)acetamide |
| 150 | 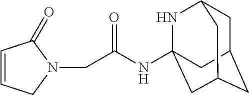 [SMILES: O=C(CN1CC=CC1=O)NC12CC3CC(CC(C3)N1)C2] | 275.16 | N-((1s,3s,5R,7S)-2-azaadamantan-1-yl)-2-(2- oxo-2,5-dihydro-1H-pyrrol-1-yl)acetamide |
| 151 | 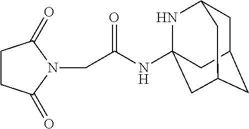 [SMILES: O=C(CN1C(=O)CCC1=O)NC12CC3CC(CC(C3)N1)C2] | 291.16 | N-((1s,3s,5R,7S)-2-azaadamantan-1-yl)-2-(2,5- dioxopyrrolidin-1-yl)acetamide |
| 152 | 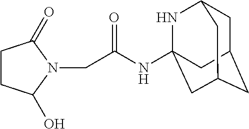 [SMILES: O=C(CN1C(=O)CCC1O)NC12CC3CC(CC(C3)N1)C2] | 293.17 | N-((1s,3s,5R,7S)-2-azaadamantan-1-yl)-2-(2- hydroxy-5-oxopyrrolidin-1-yl)acetamide |
| 153 | 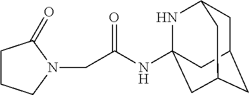 [SMILES: O=C(CN1CCCC1=O)NC12CC3CC(CC(C3)N1)C2] | 277.18 | N-((1s,3s,5R,7S)-2-azaadamantan-1-yl)-2-(2- oxopyrrolidin-1-yl)acetamide |
| 154 | 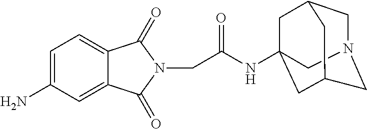 [SMILES: Nc1ccc2c(c1)C(=O)N(CC(=O)NC13CC4CC(CN(C4)C1)C3)C2=O] | 354.17 | N-((1r,3r,5R,7S)-1-azaadamantan-3-yl)-2-(5- amino-1,3-dioxoisoindolin-2-yl)acetamide |
| 155 | 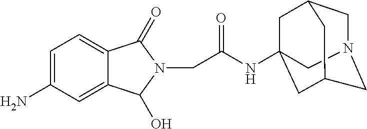 [SMILES: Nc1ccc2c(c1)C(O)N(CC(=O)NC13CC4CC(CN(C4)C1)C3)C2=O] | 356.18 | N-((1r,3r,5R,7S)-1-azaadamantan-3-yl)-2-(5- amino-3-hydroxy-1-oxoisoindolin-2- yl)acetamide |
| 156 | 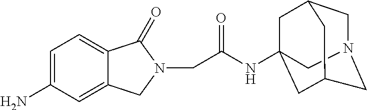 [SMILES: Nc1ccc2c(c1)CN(CC(=O)NC13CC4CC(CN(C4)C1)C3)C2=O] | 340.19 | N-((1r,3r,5R,7S)-1-azaadamantan-3-yl)-2-(5- amino-1-oxoisoindolin-2-yl)acetamide |
| 157 | 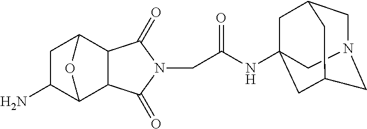 [SMILES: NC1CC2OC1C1C(=O)N(CC(=O)NC34CC5CC(CN(C5)C3)C4)C(=O)C21] | 374.20 | N-((1r,3r,5R,7S)-1-azaadamantan-3-yl)-2-(5- amino-1,3-dioxohexahydro-1H-4,7- epoxyisoindol-2(3H)-yl)acetamide |
| 158 | 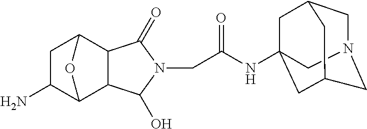 [SMILES: NC1CC2OC1C1C2C(=O)N(CC(=O)NC23CC4CC(CN(C4)C2)C3)C1O] | 376.21 | N-((1r,3r,5R,7S)-1-azaadamantan-3-yl)-2-(5- amino-3-hydroxy-1-oxohexahydro-1H-4,7- epoxyisoindol-2(3H)-yl)acetamide |
| 159 | 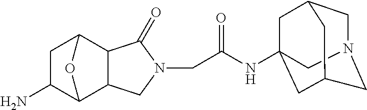 [SMILES: NC1CC2OC1C1CN(CC(=O)NC34CC5CC(CN(C5)C3)C4)C(=O)C21] | 360.22 | N-((1r,3r,5R,7S)-1-azaadamantan-3-yl)-2-(5- amino-1-oxohexahydro-1H-4,7-epoxyisoindol- 2(3H)-yl)acetamide |
| 160 | 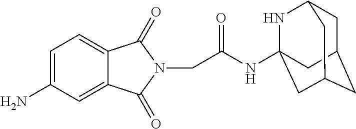 [SMILES: Nc1ccc2c(c1)C(=O)N(CC(=O)NC13CC4CC(CC(C4)N1)C3)C2=O] | 354.17 | N-((1s,3s,5R,7S)-2-azaadamantan-1-yl)-2-(5- amino-1,3-dioxoisoindolin-2-yl)acetamide |
| 161 | 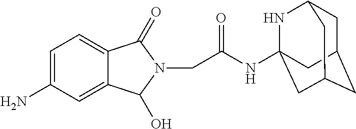 [SMILES: Nc1ccc2c(c1)C(O)N(CC(=O)NC13CC4CC(CC(C4)N1)C3)C2=O] | 356.18 | N-((1s,3s,5R,7S)-2-azaadamantan-1-yl)-2-(5- amino-3-hydroxy-1-oxoisoindolin-2- yl)acetamide |
| 162 | 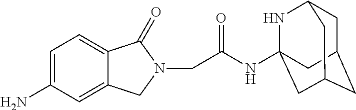 [SMILES: Nc1ccc2c(c1)CN(CC(=O)NC13CC4CC(CC(C4)N1)C3)C2=O] | 340.19 | N-((1s,3s,5R,7S)-2-azaadamantan-1-yl)-2-(5- amino-1-oxoisoindolin-2-yl)acetamide |
| 163 | 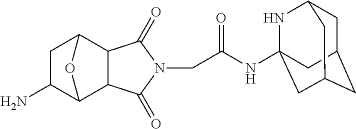 [SMILES: NC1CC2OC1C1C(=O)N(CC(=O)NC34CC5CC(CC(C5)N3)C4)C(=O)C21] | 374.20 | N-((1s,3s,5R,7S)-2-azaadamantan-1-yl)-2-(5- amino-1,3-dioxohexahydro-1H-4,7- epoxyisoindol-2(3H)-yl)acetamide |
| 164 | 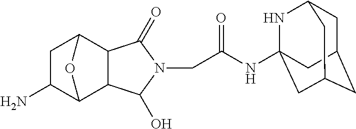 [SMILES: NC1CC2OC1C1C2C(=O)N(CC(=O)NC23CC4CC(CC(C4)N2)C3)C1O] | 376.21 | N-((1s,3s,5R,7S)-2-azaadamantan-1-yl)-2-(5- amino-3-hydroxy-1-oxohexahydro-1H-4,7- epoxyisoindol-2(3H)-yl)acetamide |
| 165 | 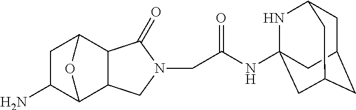 [SMILES: NC1CC2OC1C1CN(CC(=O)NC34CC5CC(CC(C5)N3)C4)C(=O)C21] | 360.22 | N-((1s,3s,5R,7S)-2-azaadamantan-1-yl)-2-(5- amino-1-oxohexahydro-1H-4,7-epoxyisoindol- 2(3H)-yl)acetamide |
| 166 | 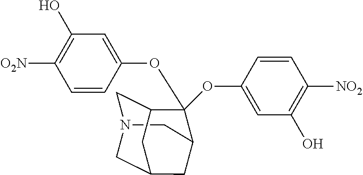 [SMILES: O=[N+]([O-])c1ccc(OC2(Oc3ccc([N+](=O)[O-])c(O)c3)C3CC4CC2CN(C4)C3)cc1O] | 443.13 | 5,5′-((1r,3R,5S,7s)-1-azaadamantane-4,4- diylbis(oxy))bis(2-nitrophenol) |
| 167 | 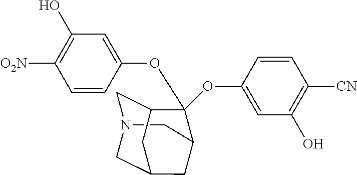 [SMILES: N#Cc1ccc(OC2(Oc3ccc([N+](=O)[O-])c(O)c3)C3CC4CC2CN(C4)C3)cc1O] | 423.14 | 2-hydroxy-4-(((1r,3R,4r,5S,7s)-4-(3-hydroxy- 4-nitrophenoxy)-1-azaadamantan-4- yl)oxy)benzonitrile |
| 168 | 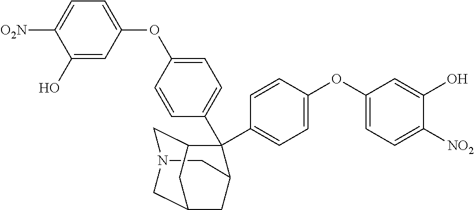 [SMILES: O=[N+]([O-])c1ccc(Oc2ccc(C3(c4ccc(Oc5ccc([N+](=O)[O-])c(O)c5)cc4)C4CC5CC3CN(C5)C4)cc2)cc1O] | 595.20 | 5,5′-(((1s,3R,5S,7r)-1-azaadamantane-4,4- diylbis(4,1-phenylene))bis(oxy))bis(2- nitrophenol) |
| 169 | 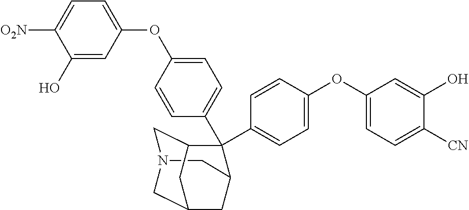 [SMILES: N#Cc1ccc(Oc2ccc(C3(c4ccc(Oc5ccc([N+](=O)[O-])c(O)c5)cc4)C4CC5CC3CN(C5)C4)cc2)cc1O] | 575.21 | 2-hydroxy-4-(4-((1s,3R,4s,5S,7r)-4-(4-(3- hydroxy-4-nitrophenoxy)phenyl)-1- azaadamantan-4-yl)phenoxy)benzonitrile |
| 170 | 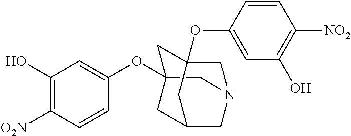 [SMILES: O=[N+]([O-])c1ccc(OC23CC4CN(C2)CC(Oc2ccc([N+](=O)[O-])c(O)c2)(C4)C3)cc1O] | 443.13 | 5,5′-((1r,3R,5S,7s)-1-azaadamantane-3,5- diylbis(oxy))bis(2-nitrophenol) |
| 171 | 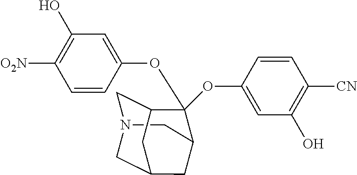 [SMILES: N#Cc1ccc(OC2(Oc3ccc([N+](=O)[O-])c(O)c3)C3CC4CC2CN(C4)C3)cc1O] | 423.14 | 2-hydroxy-4-(((1S,3R,5S,7R)-5-(3-hydroxy-4- nitrophenoxy)-1-azaadamantan-3- yl)oxy)benzonitrile |
| 172 | 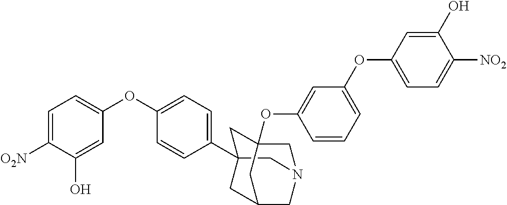 [SMILES: O=[N+]([O-])c1ccc(Oc2ccc(C34CC5CN(CC(Oc6cccc(Oc7ccc([N+](=O)[O-])c(O)c7)c6)(C5)C3)C4)cc2)cc1O] | 595.20 | 5,5′-(((1s,3R,5S,7r)-1-azaadamantane-3,5- diylbis(4,1-phenylene))bis(oxy))bis(2- nitrophenol) |
| 173 | 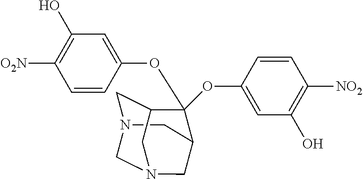 [SMILES: O=[N+]([O-])c1ccc(OC2(Oc3ccc([N+](=O)[O-])c(O)c3)C3CN4CC2CN(C3)C4)cc1O] | 444.13 | 5,5′-((1r,3r,5r,7r)-1,3-diazaadamantane-6,6- diylbis(oxy))bis(2-nitrophenol) |
| 174 | 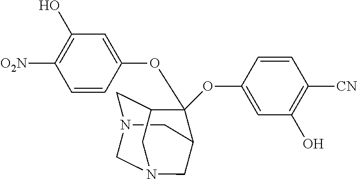 [SMILES: N#Cc1ccc(OC2(Oc3ccc([N+](=O)[O-])c(O)c3)C3CN4CC2CN(C3)C4)cc1O] | 424.14 | 2-hydroxy-4-(((1r,3r,5R,7S)-6-(3-hydroxy-4- nitrophenoxy)-1,3-diazaadamantan-6- yl)oxy)benzonitrile |
| 175 | 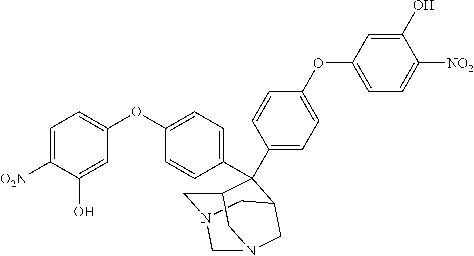 [SMILES: O=[N+]([O-])c1ccc(Oc2ccc(C3(c4ccc(Oc5ccc([N+](=O)[O-])c(O)c5)cc4)C4CN5CC3CN(C4)C5)cc2)cc1O] | 596.19 | 5,5′-(((1r,3r,5r,7r)-1,3-diazaadamantane-6,6- diylbis(4,1-phenylene))bis(oxy))bis(2- nitrophenol) |
| 176 | 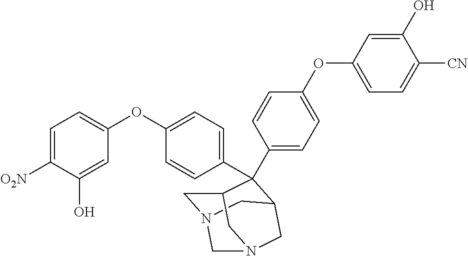 [SMILES: N#Cc1ccc(Oc2ccc(C3(c4ccc(Oc5ccc([N+](=O)[O-])c(O)c5)cc4)C4CN5CC3CN(C4)C5)cc2)cc1O] | 576.20 | 2-hydroxy-4-(4-((1r,3r,5R,7S)-6-(4-(3- hydroxy-4-nitrophenoxy)phenyl)-1,3- diazaadamantan-6-yl)phenoxy)benzonitrile |
| 177 | 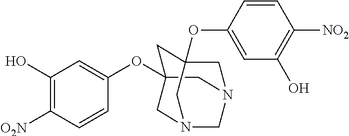 [SMILES: O=[N+]([O-])c1ccc(OC23CN4CN(C2)CC(Oc2ccc([N+](=O)[O-])c(O)c2)(C4)C3)cc1O] | 444.13 | 5,5′-((1s,3s,5s,7s)-1,3-diazaadamantane-5,7- diylbis(oxy))bis(2-nitrophenol) |
| 178 | 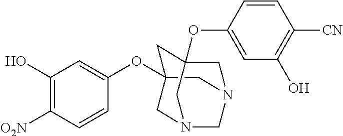 [SMILES: N#Cc1ccc(OC23CN4CN(C2)CC(Oc2ccc([N+](=O)[O-])c(O)c2)(C4)C3)cc1O] | 424.14 | 2-hydroxy-4-(((1R,3S,5r,7s)-7-(3-hydroxy-4- nitrophenoxy)-1,3-diazaadamantan-5- yl)oxy)benzonitrile |
| 179 | 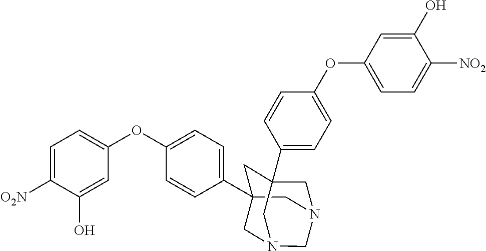 [SMILES: O=[N+]([O-])c1ccc(Oc2ccc(C34CN5CN(C3)CC(c3ccc(Oc6ccc([N+](=O)[O-])c(O)c6)cc3)(C5)C4)cc2)cc1O] | 596.19 | 5,5′-(((1s,3s,5s,7s)-1,3-diazaadamantane-5,7- diylbis(4,1-phenylene))bis(oxy))bis(2- nitrophenol) |
| 180 | 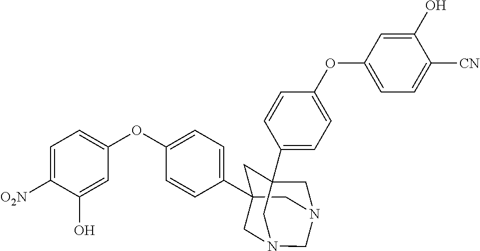 [SMILES: N#Cc1ccc(Oc2ccc(C34CN5CN(C3)CC(c3ccc(Oc6ccc([N+](=O)[O-])c(O)c6)cc3)(C5)C4)cc2)cc1O] | 576.20 | 2-hydroxy-4-(4-((1R,3S,5r,7s)-7-(4-(3- hydroxy-4-nitrophenoxy)phenyl)-1,3- diazaadamantan-5-yl)phenoxy)benzonitrile |
| 181 | 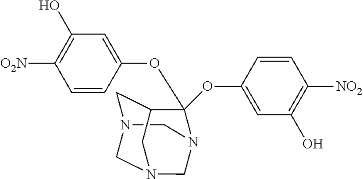 [SMILES: O=[N+]([O-])c1ccc(OC2(Oc3ccc([N+](=O)[O-])c(O)c3)C3CN4CN(C3)CN2C4)cc1O] | 445.12 | 5,5′-((1R,3S,5s,7r)-1,3,5-triazaadamantane- 6,6-diylbis(oxy))bis(2-nitrophenol) |
| 182 | 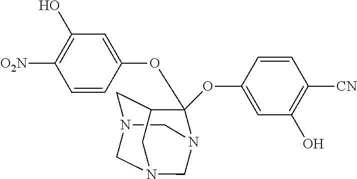 [SMILES: N#Cc1ccc(OC2(Oc3ccc([N+](=O)[O-])c(O)c3)C3CN4CN(C3)CN2C4)cc1O] | 425.13 | 2-hydroxy-4-(((1R,3S,5S,6R,7R)-6-(3- hydroxy-4-nitrophenoxy)-1,3,5- triazaadamantan-6-yl)oxy)benzonitrile |
| 183 | 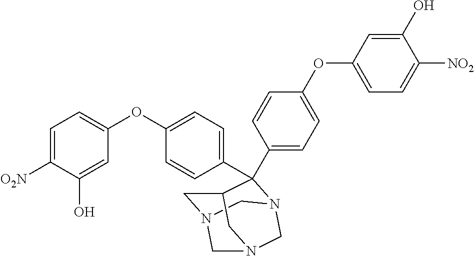 [SMILES: O=[N+]([O-])c1ccc(Oc2ccc(C3(c4ccc(Oc5ccc([N+](=O)[O-])c(O)c5)cc4)C4CN5CN(C4)CN3C5)cc2)cc1O] | 597.19 | 5,5′-(((1R,3S,5s,7r)-1,3,5-triazaadamantane- 6,6-diylbis(4,1-phenylene))bis(oxy))bis(2- nitrophenol) |
| 184 | 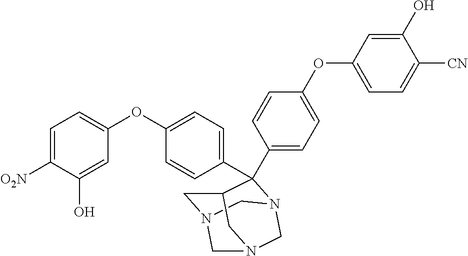 [SMILES: N#Cc1ccc(Oc2ccc(C3(c4ccc(Oc5ccc([N+](=O)[O-])c(O)c5)cc4)C4CN5CN(C4)CN3C5)cc2)cc1O] | 577.20 | 2-hydroxy-4-(4-((1R,3S,5S,6R,7R)-6-(4-(3- hydroxy-4-nitrophenoxy)phenyl)-1,3,5- triazaadamantan-6-yl)phenoxy)benzonitrile |
| 185 | 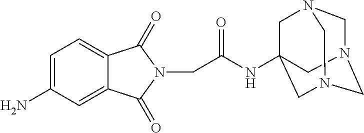 [SMILES: Nc1ccc2c(c1)C(=O)N(CC(=O)NC13CN4CN(CN(C4)C1)C3)C2=O] | 356.16 | N-((1s,3s,5s)-1,3,5-triazaadamantan-7-yl)-2- (5-amino-1,3-dioxoisoindolin-2-yl)acetamide |
| 186 | 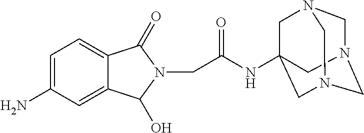 [SMILES: Nc1ccc2c(c1)C(O)N(CC(=O)NC13CN4CN(CN(C4)C1)C3)C2=O] | 358.18 | N-((1s,3s,5s)-1,3,5-triazaadamantan-7-yl)-2-(5- amino-3-hydroxy-1-oxoisoindolin-2- yl)acetamide |
| 187 | 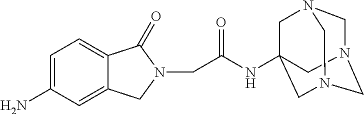 [SMILES: Nc1ccc2c(c1)CN(CC(=O)NC13CN4CN(CN(C4)C1)C3)C2=O] | 342.18 | N-((1s,3s,5s)-1,3,5-triazaadamantan-7-yl)-2-(5- amino-1-oxoisoindolin-2-yl)acetamide |
| 188 | 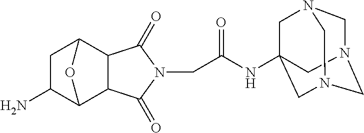 [SMILES: NC1CC2OC1C1C(=O)N(CC(=O)NC34CN5CN(CN(C5)C3)C4)C(=O)C21] | 376.19 | N-((1s,3s,5s)-1,3,5-triazaadamantan-7-yl)-2-(5- amino-1,3-dioxohexahydro-1H-4,7- epoxyisoindol-2(3H)-yl)acetamide |
| 189 | 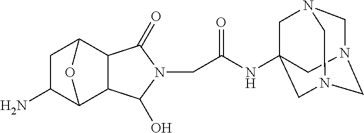 [SMILES: NC1CC2OC1C1C2C(=O)N(CC(=O)NC23CN4CN(CN(C4)C2)C3)C1O] | 378.20 | N-((1s,3s,5s)-1,3,5-triazaadamantan-7-yl)-2-(5- amino-3-hydroxy-1-oxohexahydro-1H-4,7- epoxyisoindol-2(3H)-yl)acetamide |
| 190 | 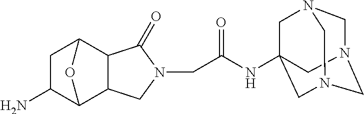 [SMILES: NC1CC2OC1C1CN(CC(=O)NC34CN5CN(CN(C5)C3)C4)C(=O)C21] | 362.21 | N-((1s,3s,5s)-1,3,5-triazaadamantan-7-yl)-2-(5- amino-1-oxohexahydro-1H-4,7-epoxyisoindol- 2(3H)-yl)acetamide |
| 191 | 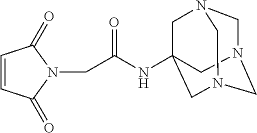 [SMILES: O=C(CN1C(=O)C=CC1=O)NC12CN3CN(CN(C3)C1)C2] | 291.13 | N-((1s,3s,5s)-1,3,5-triazaadamantan-7-yl)-2- (2,5-dioxo-2,5-dihydro-1H-pyrrol-1- yl)acetamide |
| 192 | 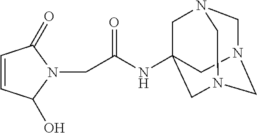 [SMILES: O=C(CN1C(=O)C=CC1O)NC12CN3CN(CN(C3)C1)C2] | 293.15 | N-((1s,3s,5s)-1,3,5-triazaadamantan-7-yl)- 2-(2-hydroxy-5-oxo-2,5-dihydro-1H-pyrrol-1- yl)acetamide |
| 193 | 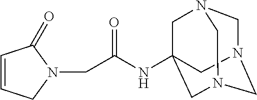 [SMILES: O=C(CN1CC=CC1=O)NC12CN3CN(CN(C3)C1)C2] | 277.15 | N-((1s,3s,5s)-1,3,5-triazaadamantan-7-yl)-2-(2- oxo-2,5-dihydro-1H-pyrrol-1-yl)acetamide |
| 194 | 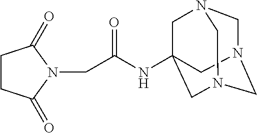 [SMILES: O=C(CN1C(=O)CCC1=O)NC12CN3CN(CN(C3)C1)C2] | 293.15 | N-((1s,3s,5s)-1,3,5-triazaadamantan-7-yl)- 2-(2,5-dioxopyrrolidin-1-yl)acetamide |
| 195 | 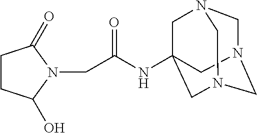 [SMILES: O=C(CN1C(=O)CCC1O)NC12CN3CN(CN(C3)C1)C2] | 295.16 | N-((1s,3s,5s)-1,3,5-triazaadamantan-7-yl)- 2-(2-hydroxy-5-oxopyrrolidin-1-yl)acetamide |
| 196 | 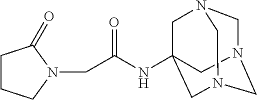 [SMILES: O=C(CN1CCCC1=O)NC12CN3CN(CN(C3)C1)C2] | 279.17 | N-((1s,3s,5s)-1,3,5-triazaadamantan-7-yl)-2-(2- oxopyrrolidin-1-yl)acetamide |
| 197 | 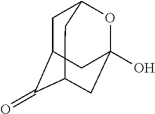 [SMILES: O=C1C2CC3CC1CC(O)(C2)O3] | 168.08 | (1r,3s,5R,7S)-1-hydroxy-2-oxaadamantan- 6-one |
| 198 | 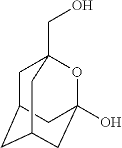 [SMILES: OCC12CC3CC(CC(O)(C3)O1)C2] | 184.11 | (1r,3s,5R,7S)-3-(hydroxymethyl)-2- oxaadamantan-1-ol |
| 199 | 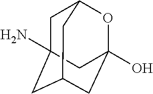 [SMILES: NC12CC3CC(C1)OC(O)(C3)C2] | 169.11 | (1S,3R,5R,7R)-5-amino-2-oxaadamantan- 1-ol |
| 200 | 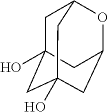 [SMILES: OC12CC3CC(O)(CC(C1)O3)C2] | 170.09 | (1r,3r,5s,7s)-2-oxaadamantane-5,7-diol |
| 201 | 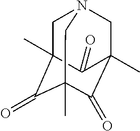 [SMILES: CC12CN3CC(C)(C1=O)C(=O)C(C)(C3)C2=O] | 221.11 | (3s,5s,7s)-3,5,7-trimethyl-1-azaadamantane- 4,6,10-trione |
| 202 | 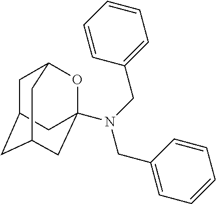 [SMILES: c1ccc(CN(Cc2ccccc2)C23CC4CC(CC(C4)O2)C3)cc1] | 333.21 | (1r,3s,5R,7S)-N,N-dibenzyl-2-oxaadamantan- 1-amine |
| 203 | 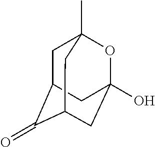 | 182.09 | (1r,3s,5R,7S)-1-hydroxy-3-methyl-2- oxaadamantan-6-one |
| 204 | 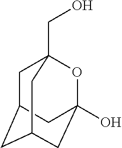 [SMILES: OCC12CC3CC(CC(O)(C3)O1)C2] | 184.11 | (1r,3s,5R,7S)-3-(hydroxymethyl)-2- oxaadamantan-1-ol |
| 205 | 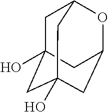 [SMILES: OC12CC3CC(O)(CC(C1)O3)C2] | 170.09 | (1r,3r,5s,7s)-2-oxaadamantane-5,7-diol |
| 206 | 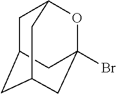 [SMILES: BrC12CC3CC(CC(C3)O1)C2] | 216.01 | (1s,3s,5R,7S)-1-bromo-2-oxaadamantane |
| 207 | 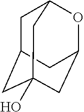 [SMILES: OC12CC3CC(C1)OC(C3)C2] | 154.10 | (1R,3S,5s,7s)-2-oxaadamantan-5-ol |
| 208 | 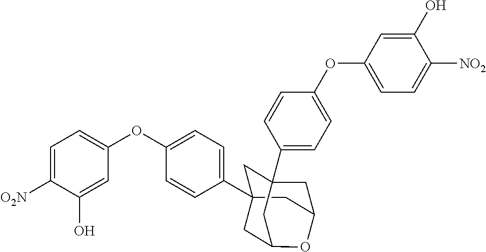 [SMILES: O=[N+]([O-])c1ccc(Oc2ccc(C34CC5CC(c6ccc(Oc7ccc([N+](=O)[O-])c(O)c7)cc6)(CC(C3)O5)C4)cc2)cc1O] | 596.18 | 5,5′-(((1s,3s,5s,7s)-2-oxaadamantane-5,7- diylbis(4,1-phenylene))bis(oxy))bis(2- nitrophenol) |
| 209 | 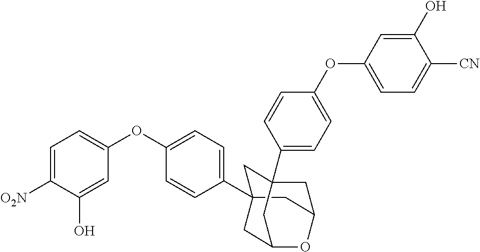 [SMILES: N#Cc1ccc(Oc2ccc(C34CC5CC(c6ccc(Oc7ccc([N+](=O)[O-])c(O)c7)cc6)(CC(C3)O5)C4)cc2)cc1O] | 576.19 | 2-hydroxy-4-(4-((1R,3S,5r,7s)-7-(4-(3- hydroxy-4-nitrophenoxy)phenyl)-2- oxaadamantan-5-yl)phenoxy)benzonitrile |
| 210 | 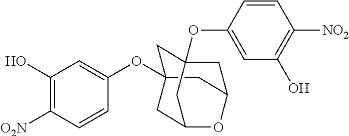 [SMILES: O=[N+]([O-])c1ccc(OC23CC4CC(Oc5ccc([N+](=O)[O-])c(O)c5)(CC(C2)O4)C3)cc1O] | 444.12 | 5,5′-((1r,3r,5s,7s)-2-oxaadamantane-5,7- diylbis(oxy))bis(2-nitrophenol) |
| 211 | 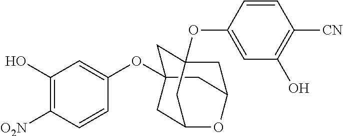 [SMILES: N#Cc1ccc(OC23CC4CC(Oc5ccc([N+](=O)[O-])c(O)c5)(CC(C2)O4)C3)cc1O] | 424.13 | 2-hydroxy-4-(((1R,3S,5r,7s)-7-(3-hydroxy-4- nitrophenoxy)-2-oxaadamantan-5- yl)oxy)benzonitrile |
| 212 | 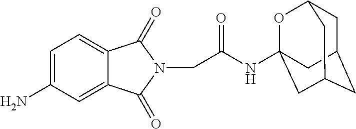 [SMILES: Nc1ccc2c(c1)C(=O)N(CC(=O)NC13CC4CC(CC(C4)O1)C3)C2=O] | 355.15 | N-((1r,3s,5R,7S)-2-oxaadamantan-1-yl)-2-(5- amino-1,3-dioxoisoindolin-2-yl)acetamide |
| 213 | 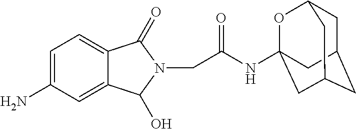 [SMILES: Nc1ccc2c(c1)C(O)N(CC(=O)NC13CC4CC(CC(C4)O1)C3)C2=O] | 357.17 | N-((1r,3s,5R,7S)-2-oxaadamantan-1-yl)-2-(5- amino-3-hydroxy-1-oxoisoindolin-2- yl)acetamide |
| 214 | 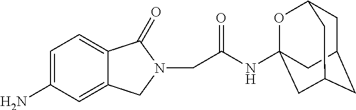 [SMILES: Nc1ccc2c(c1)CN(CC(=O)NC13CC4CC(CC(C4)O1)C3)C2=O] | 341.17 | N-((1r,3s,5R,7S)-2-oxaadamantan-1-yl)-2-(5- amino-1-oxoisoindolin-2-yl)acetamide |
| 215 | 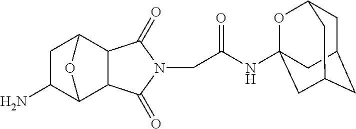 [SMILES: NC1CC2OC1C1C(=O)N(CC(=O)NC34CC5CC(CC(C5)O3)C4)C(=O)C21] | 357.18 | N-((1r,3s,5R,7S)-2-oxaadamantan-1-yl)-2-(5- amino-1,3-dioxohexahydro-1H-4,7- epoxyisoindol-2(3H)-yl)acetamide |
| 216 | 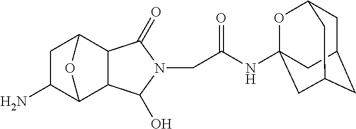 [SMILES: NC1CC2OC1C1C2C(=O)N(CC(=O)NC23CC4CC(CC(C4)O2)C3)C1O] | 377.20 | N-((1r,3s,5R,7S)-2-oxaadamantan-1-yl)-2-(5- amino-3-hydroxy-1-oxohexahydro-1H-4,7- epoxyisoindol-2(3H)-yl)acetamide |
| 217 | 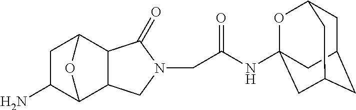 [SMILES: NC1CC2OC1C1CN(CC(=O)NC34CC5CC(CC(C5)O3)C4)C(=O)C21] | 361.20 | N-((1r,3s,5R,7S)-2-oxaadamantan-1-yl)-2-(5- amino-1-oxohexahydro-1H-4,7-epoxyisoindol- 2(3H)-yl)acetamide |
| 218 | 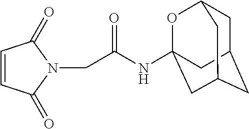 [SMILES: O=C(CN1C(=O)C=CC1=O)NC12CC3CC(CC(C3)O1)C2] | 290.13 | N-((1r,3s,5R,7S)-2-oxaadamantan-1-yl)- 2-(2,5-dioxo-2,5-dihydro-1H-pyrrol-1- yl)acetamide |
| 219 | 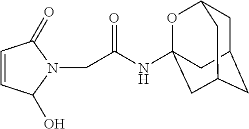 [SMILES: O=C(CN1C(=O)C=CC1O)NC12CC3CC(CC(C3)O1)C2] | 292.14 | N-((1r,3s,5R,7S)-2-oxaadamantan-1-yl)-2-(2- hydroxy-5-oxo-2,5-dihydro-1H-pyrrol-1- yl)acetamide |
| 220 | 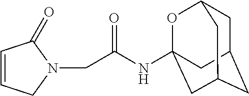 [SMILES: O=C(CN1CC=CC1=O)NC12CC3CC(CC(C3)O1)C2] | 276.15 | N-((1r,3s,5R,7S)-2-oxaadamantan-1-yl)-2-(2- oxo-2,5-dihydro-1H-pyrrol-1-yl)acetamide |
| 221 | 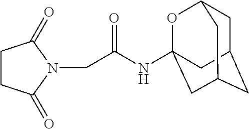 [SMILES: O=C(CN1C(=O)CCC1=O)NC12CC3CC(CC(C3)O1)C2] | 292.14 | N-((1r,3s,5R,7S)-2-oxaadamantan-1-yl)-2-(2,5- dioxopyrrolidin-1-yl)acetamide |
| 222 | 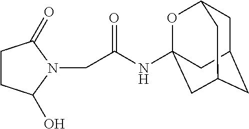 [SMILES: O=C(CN1C(=O)CCC1O)NC12CC3CC(CC(C3)O1)C2] | 394.16 | N-((1r,3s,5R,7S)-2-oxaadamantan-1-yl)-2-(2- hydroxy-5-oxopyrrolidin-1-yl)acetamide |
| 223 | 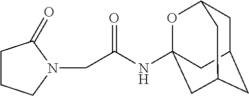 [SMILES: O=C(CN1CCCC1=O)NC12CC3CC(CC(C3)O1)C2] | 278.16 | N-((1r,3s,5R,7S)-2-oxaadamantan-1-yl)-2-(2- oxopyrrolidin-1-yl)acetamide |
| 224 | 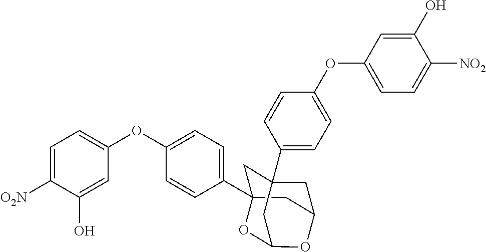 [SMILES: O=[N+]([O-])c1ccc(Oc2ccc(C34CC5CC(c6ccc(Oc7ccc([N+](=O)[O-])c(O)c7)cc6)(C3)OC(C4)O5)cc2)cc1O] | 598.16 | 5,5′-(((1R,3S,5S,7S)-2,4-dioxaadamantane-1,7- diylbis(4,1-phenylene))bis(oxy))bis(2- nitrophenol) |
| 225 | 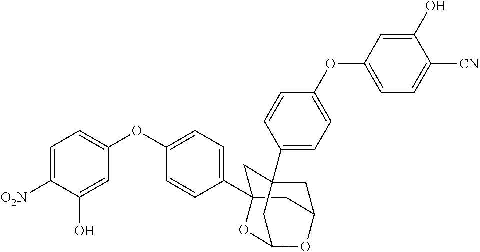 [SMILES: N#Cc1ccc(Oc2ccc(C34CC5CC(c6ccc(Oc7ccc([N+](=O)[O-])c(O)c7)cc6)(C3)OC(C4)O5)cc2)cc1O] | 578.17 | 2-hydroxy-4-(4-((1R,3S,5S,7S)-1-(4-(3- hydroxy-4-nitrophenoxy)phenyl)-2,4- dioxaadamantan-7-yl)phenoxy)benzonitrile |
| 226 | 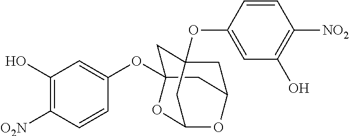 [SMILES: O=[N+]([O-])c1ccc(OC23CC4CC(Oc5ccc([N+](=O)[O-])c(O)c5)(C2)OC(C3)O4)cc1O] | 446.10 | 5,5′-((1S,3S,5S,7R)-2,4-dioxaadamantane-1,7- diylbis(oxy))bis(2-nitrophenol) |
| 227 | 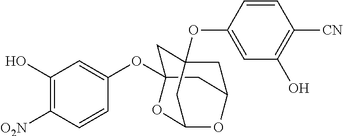 [SMILES: N#Cc1ccc(OC23CC4CC(Oc5ccc([N+](=O)[O-])c(O)c5)(C2)OC(C3)O4)cc1O] | 426.11 | 2-hydroxy-4-(((1S,3S,5S,7R)-1-(3-hydroxy-4- nitrophenoxy)-2,4-dioxaadamantan-7- yl)oxy)benzonitrile |
| 228 | 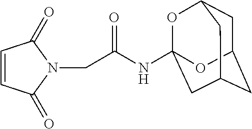 [SMILES: O=C(CN1C(=O)C=CC1=O)NC12CC3CC(CC(C3)O1)O2] | 292.11 | N-((1R,3r,5S,7r)-2,4-dioxaadamantan-3-yl)-2- (2,5-dioxo-2,5-dihydro-1H-pyrrol-1- yl)acetamide |
| 229 | 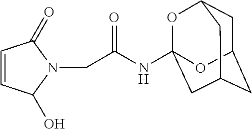 [SMILES: O=C(CN1C(=O)C=CC1O)NC12CC3CC(CC(C3)O1)O2] | 294.12 | N-((1R,3r,5S,7r)-2,4-dioxaadamantan-3-yl)- 2-(2-hydroxy-5-oxo-2,5-dihydro-1H-pyrrol-1- yl)acetamide |
| 230 | 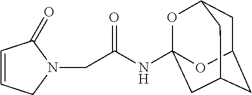 [SMILES: O=C(CN1CC=CC1=O)NC12CC3CC(CC(C3)O1)O2] | 278.13 | N-((1R,3r,5S,7r)-2,4-dioxaadamantan-3-yl)- 2-(2-oxo-2,5-dihydro-1H-pyrrol-1-yl)acetamide |
| 231 | 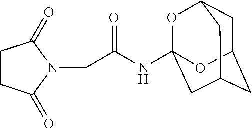 [SMILES: O=C(CN1C(=O)CCC1=O)NC12CC3CC(CC(C3)O1)O2] | 294.12 | N-((1R,3r,5S,7r)-2,4-dioxaadamantan-3-yl)-2- (2,5-dioxopyrrolidin-1-yl)acetamide |
| 232 | 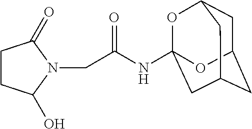 [SMILES: O=C(CN1C(=O)CCC1O)NC12CC3CC(CC(C3)O1)O2] | 296.14 | N-((1R,3r,5S,7r)-2,4-dioxaadamantan-3-yl)- 2-(2-hydroxy-5-oxopyrrolidin-1-yl)acetamide |
| 233 | 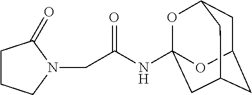 [SMILES: O=C(CN1CCCC1=O)NC12CC3CC(CC(C3)O1)O2] | 280.14 | N-((1R,3r,5S,7r)-2,4-dioxaadamantan-3-yl)- 2-(2-oxopyrrolidin-1-yl)acetamide |
| 234 | 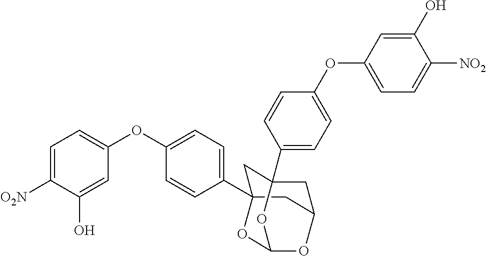 [SMILES: O=[N+]([O-])c1ccc(Oc2ccc(C34CC5CC(c6ccc(Oc7ccc([N+](=O)[O-])c(O)c7)cc6)(C3)OC(O5)O4)cc2)cc1O] | 600.14 | 5,5′-(((1R,3s,5S,7r)-2,4,10-trioxaadamantane- 1,5-diylbis(4,1-phenylene))bis(oxy))bis(2- nitrophenol) |
| 235 | 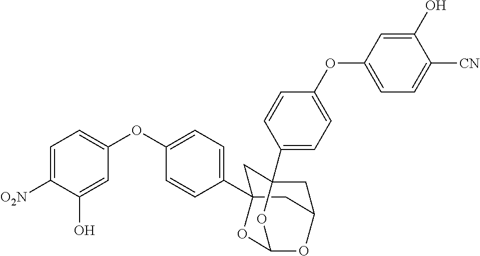 [SMILES: N#Cc1ccc(Oc2ccc(C34CC5CC(c6ccc(Oc7ccc([N+](=O)[O-])c(O)c7)cc6)(C3)OC(O5)O4)cc2)cc1O] | 580.15 | 2-hydroxy-4-(4-((1R,3R,5S,7S)-5-(4-(3- hydroxy-4-nitrophenoxy)phenyl)-2,4,10- trioxaadamantan-1-yl)phenoxy)benzonitrile |
| 236 | 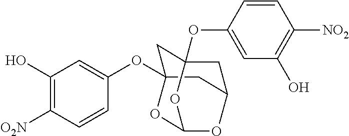 [SMILES: O=[N+]([O-])c1ccc(OC23CC4CC(Oc5ccc([N+](=O)[O-])c(O)c5)(C2)OC(O4)O3)cc1O] | 448.08 | 5,5′-((1R,3s,5S,7r)-2,4,10-trioxaadamantane- 1,5-diylbis(oxy))bis(2-nitrophenol) |
| 237 | 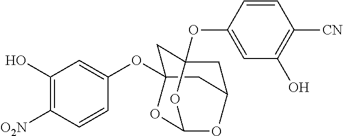 [SMILES: N#Cc1ccc(OC23CC4CC(Oc5ccc([N+](=O)[O-])c(O)c5)(C2)OC(O4)O3)cc1O] | 428.09 | 2-hydroxy-4-(((1R,3R,5S,7S)-5-(3-hydroxy- 4-nitrophenoxy)-2,4,10-trioxaadamantan-1- yl)oxy)benzonitrile |
| 238 | 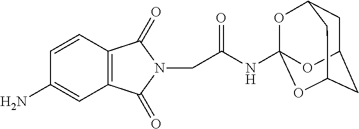 [SMILES: Nc1ccc2c(c1)C(=O)N(CC(=O)NC13OC4CC(CC(C4)O1)O3)C2=O] | 359.11 | N-((1s,5s,7s)-2,4,10-trioxaadamantan-3-yl)- 2-(5-amino-1,3-dioxoisoindolin-2-yl)acetamide |
| 239 | 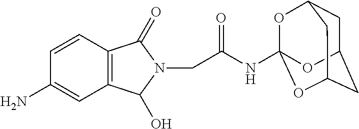 [SMILES: Nc1ccc2c(c1)C(O)N(CC(=O)NC13OC4CC(CC(C4)O1)O3)C2=O] | 361.13 | N-((1s,5s,7s)-2,4,10-trioxaadamantan-3-yl)- 2-(5-amino-3-hydroxy-1-oxoisoindolin-2- yl)acetamide |
| 240 | 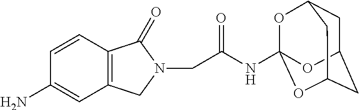 [SMILES: Nc1ccc2c(c1)CN(CC(=O)NC13OC4CC(CC(C4)O1)O3)C2=O] | 345.13 | N-((1s,5s,7s)-2,4,10-trioxaadamantan-3-yl)- 2-(5-amino-1-oxoisoindolin-2-yl)acetamide |
| 241 | 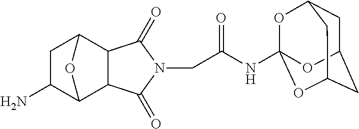 [SMILES: NC1CC2OC1C1C(=O)N(CC(=O)NC34OC5CC(CC(C5)O3)O4)C(=O)C21] | 379.14 | N-((1s,5s,7s)-2,4,10-trioxaadamantan-3-yl)- 2-(5-amino-1,3-dioxohexahydro-1H-4,7- epoxyisoindol-2(3H)-yl)acetamide |
| 242 | 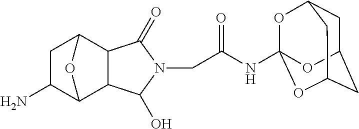 [SMILES: NC1CC2OC1C1C2C(=O)N(CC(=O)NC23OC4CC(CC(C4)O2)O3)C1O] | 381.15 | N-((1s,5s,7s)-2,4,10-trioxaadamantan-3-yl)- 2-(5-amino-3-hydroxy-1-oxohexahydro-1H- 4,7-epoxyisoindol-2(3H)-yl)acetamide |
| 243 | 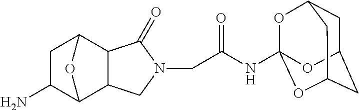 [SMILES: NC1CC2OC1C1CN(CC(=O)NC34OC5CC(CC(C5)O3)O4)C(=O)C21] | 365.16 | N-((1s,5s,7s)-2,4,10-trioxaadamantan-3-yl)- 2-(5-amino-1-oxohexahydro-1H-4,7- epoxyisoindol-2(3H)-yl)acetamide |
| 244 | 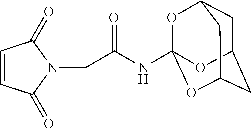 [SMILES: O=C(CN1C(=O)C=CC1=O)NC12OC3CC(CC(C3)O1)O2] | 294.09 | N-((1s,5s,7s)-2,4,10-trioxaadamantan-3-yl)- 2-(2,5-dioxo-2,5-dihydro-1H-pyrrol-1- yl)acetamide |
| 245 | 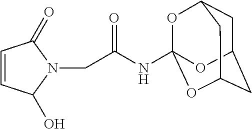 [SMILES: O=C(CN1C(=O)C=CC1O)NC12OC3CC(CC(C3)O1)O2] | 296.10 | N-((1s,5s,7s)-2,4,10-trioxaadamantan-3-yl)- 2-(2-hydroxy-5-oxo-2,5-dihydro-1H-pyrrol-1- yl)acetamide |
| 246 | 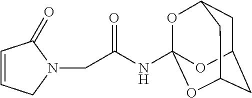 [SMILES: O=C(CN1CC=CC1=O)NC12OC3CC(CC(C3)O1)O2] | 280.11 | N-((1s,5s,7s)-2,4,10-trioxaadamantan-3-yl)- 2-(2-oxo-2,5-dihydro-1H-pyrrol-1-yl)acetamide |
| 247 | 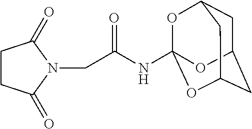 [SMILES: O=C(CN1C(=O)CCC1=O)NC12OC3CC(CC(C3)O1)O2] | 296.10 | N-((1s,5s,7s)-2,4,10-trioxaadamantan-3-yl)-2- (2,5-dioxopyrrolidin-1-yl)acetamide |
| 248 | 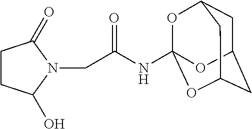 [SMILES: O=C(CN1C(=O)CCC1O)NC12OC3CC(CC(C3)O1)O2] | 298.12 | N-((1s,5s,7s)-2,4,10-trioxaadamantan-3-yl)- 2-(2-hydroxy-5-oxopyrrolidin-1-yl)acetamide |
| 249 | 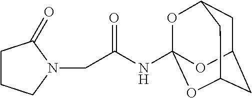 [SMILES: O=C(CN1CCCC1=O)NC12OC3CC(CC(C3)O1)O2] | 282.12 | N-((1s,5s,7s)-2,4,10-trioxaadamantan-3-yl)- 2-(2-oxopyrrolidin-1-yl)acetamide |

### Preparation of Injection

### Example 250

### Preparation of Injection 1

Compound 2 (example 2) 8.0 g, DMSO 50 ml, 1,2-propanediol 500 ml and Tween 80 100 ml were dissolved and the injection water was added up to total volume of 5000 ml. The solution was filtered with 0.22 μm membrane filter and sterilized for 30 min at 100° C. to obtain 1000 preparation of injection 8 mg/5 ml.

### BIOLOGICAL ACTIVITY

### Example 251

### In Vitro Anti-Cancer Cell Experiment

Methods:

a. Cell lines: Human pancreatic cancer cell line Panc-1, human colorectal cancer cell line HT₂₉ and human lung cancer cell line NCI-H₄₆₀; the medium: s DMEM (Gibco BRL), containing 10% fetal calf serum (Gibco BRL) and L-glutamine (Gibco BRL) 2 mmol. b. Test samples: example compounds 37, 43, 47, 62 and 68. The samples were dissolved in dimethyl sulfoxide (DMS O, Sigma, United States) and medium was added to the final concentration of 0.5%. Cisplatin was as positive control of (CDDP, purity 96%, from Kunming Institute of Precious Metals). c. Method: cells were digested with trypsin and dispersed into single cells in the medium containing penicillin (25 U/ml) and streptomycin (25 μg/ml). The cells were seeded in 96-well culture plates (Corning Incorporated), at 37° C., in a humidifield atmosphere with 5% CO₂ present for 24 hours. The culture medium was removed, 1-100 μm test compounds were added, cultured for 48 hours. Culture medium was removed and thiophene Wow blue (MTT, USA Sigma products) was added. The result was assayed by 5K601-based microplate reader (Japan Seikagaku company's products), 570 nm/630 nm optical density (OD). Calculation of cell viability: (Experimental group OD/control OD)×100%; Positive control CDDP was treated in the same way.

Results:

Inhibition of colorectal cancer: as shown in table 2 five example compounds of 68 and 62 showed significant effect of anti-proliferate on HT₂₉ at low IC₅₀ values, respectively, 1.03 μg/ml (P<0.05) and 3.62 μg/ml (P<0.05) than conventional 5-FU and Cisplatin. The rest example compounds of 37, 47 and 43 showed certain effect of anti-proliferate on HT₂₉ at low IC₅₀ values, respectively, 35.62 μg/ml, 38.33 μg/ml and 54.12 μg/ml than CDDP and Cisplatin.

Inhibition of Breast Cancer Cells: five example compounds of 68, 62, 37, 47 and 43 showed certain effect of anti-proliferate on MCF7 cells at low IC₅₀ values, respectively, 2.28 μg/ml, 6.94 μg/ml, 19.26 μg/ml, 56.32 μg/ml and 44.23 μg/ml than CDDP and Cisplatin. The example compound 68 showed more sensitive to NCI H-460 cells IC₅₀ (P<0.05).

Inhibition of pancreatic cancer: as shown in table 2 five example compounds 68, 62, 37, 47 and 43 showed anti-proliferative effect on Panc-1 cells at low IC₅₀ values, respectively, 3.4 μg/ml, 3.26 μg/ml, 5.23 μg/ml, 17.6 μg/ml and 26.8 μg/ml than CDDP and Cisplatin. The activity of example compound 62 is close to conventional 5-FU as good data as 5-FU.

TABLE 2
Growth Inhibition of cell line IC₅₀ (nM)

| Example | HT₂₉ | MCF7 | Panc-1 | NCI-H460 |
|---|---|---|---|---|
| 37 | 35.62 | 19.26 | 5.23 | 7.73 |
| 43 | 54.12 | 44.23 | 26.8 | 13.65 |
| 47 | 38.33 | 56.32 | 17.6 | 17.25 |
| 62 | 3.62 | 6.94 | 3.26 | 4.89 |
| 68 | 1.03 | 2.28 | 3.4 | 3.38 |
| CDDP | 3.69 | 3.92 | 2.17 | 5.40 |
| 5-FU | 14.36 | | 3.33 | |

Inhibition of lung cancer: as shown in table 2 five example compounds 68, 62, 37, 47 and 43 showed anti-proliferative effect on NCI-H₄₆₀ cells at low IC₅₀ values, respectively, 3.38 μg/ml, 4.89 μg/ml, 7.73 μg/ml, 17.25 μg/ml and 13.65 μg/ml.

### Example 252

### Efficacy Studies of Anti-Tumor In Vivo

Test Samples:

example compounds 1, 7, 8, 9, 10, 11, 15, 16, 27, 29, 30, 32, 34, 35, 36, 37, 38, 41, 42, 43, 44, 47, 49, 50, 51, 53, 54, 55, 56, 57, 58, 59, 60, 61, 62, 64, 65, 66, 67, 68, 69, 70, 71, 73, 74, 75, 76, 78, 87, 88, 91, 93, 94, 97, 98, 100, 105 and 107 (see Table 1).

Test Animals:

Kunming healthy mice, weighing 19˜21 g, male and female groups, each 10 by the Beijing Military Academy of Medical Sciences Animal Center. Tumor strain: mouse ascites sarcoma S180 is passaged from the Beijing Military Academy of Medical Sciences.

Methods:

Xenografts cultured S₁₈₀ tumor cells were implanted subcutaneously into the flank region of mice and tumors were allowed to grow to the desired average size of 100 mg. The mice were randomized into control and treatment groups with 10 mice per group. The control group was injected with the vehicle used to dissolve the drug. Other groups received the test compounds example compound and positive group, cyclophosphamide (CTX) and 5-fluorouracil (5-FU)) at the dose and schedule as indicated in Table 3. Tumor measurements were taken every other day 20% tumor growth inhibition which was not statistically significant.

Results:

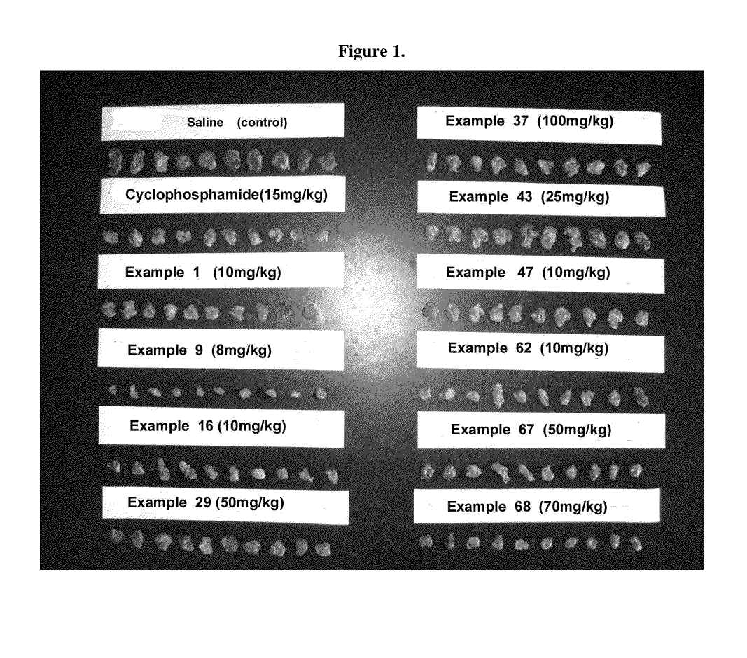

the in vivo experimental data showed the compound 7, 8, 9, 11, 16, 68 and 107 was a significant difference by comparation with the control group (p<0.05) in the test results shown in Table 3 and FIG. 1.

TABLE 3
Growth Inhibition of S₁₈₀ sarcoma ( X̅ ± SD, n = 16)

| | | | | |
|---|---|---|---|---|
| Control | | — | 2.02 ± 0.37 | — |
| CTX | 15 | iv | 1.21 ± 0.88 | 44.5 |
| 1 | 10 | ip | 1.24 ± 0.31 | 41.34 |
| 7 | 10 | iv | 0.75 ± 0.51 | 53.33** |
| 8 | 20 | iv | 0.59 ± 0.35 | 52.98** |
| 9 | 8 | ip | 0.96 ± 0.60 | 58** |
| 10 | 50 | iv | 1.53 ± 0.34 | 42.11 |
| 11 | 80 | ip | 1.11 ± 0.33 | 54** |
| 15 | 25 | iv | 0.86 ± 0.47 | 32.08 |
| 16 | 10 | iv | 0.55 ± 0.44 | 52.9** |
| 27 | 16 | ip | 1.43 ± 0.72 | 10.75 |
| 29 | 50 | ip | 1.66 ± 0.13 | 32 |
| 30 | 100 | ip | 1.76 ± 1.11 | 37.61 |
| 32 | 100 | iv | 1.18 ± 0.30 | 33.03 |
| 34 | 12.5 | iv | 1.54 ± 0.47 | 41.69 |
| 35 | 100 | ip | 1.47 ± 0.26 | 40.56 |
| 36 | 50 | ip | 1.63 ± 0.86 | 32 |
| 37 | 100 | iv | 1.54 ± 0.35 | 44.75 |
| 38 | 100 | ip | 1.63 ± 0.69 | 32 |
| 41 | 400 | ip | 1.98 ± 0.11 | 26.1 |
| 42 | 50 | iv | 1.56 ± 0.47 | 32.12 |
| 43 | 25 | iv | 1.96 ± 0.40 | 24.19 |
| 44 | 2 | iv | 1.48 ± 0.77 | 35 |
| 47 | 10 | iv | 1.51 ± 0.32 | 34.72 |
| 49 | 50 | iv | 1.54 ± .033 | 34.35 |
| 50 | 150 | ip | 1.54 ± 0.42 | 47 |
| 51 | 100 | ip | 1.44 ± 0.39 | 44.81* |
| 53 | 40 | iv | 1.46 ± 0.50 | 40.46 |
| 54 | 100 | iv | 1.95 ± 0.33 | 24.87 |
| 55 | 100 | ip | 1.57 ± 0.49 | 32 |
| 56 | 10 | ip | 1.43 ± 0.76 | 41 |
| 57 | 40 | ip | 1.84 ± 0.45 | 30.46 |
| 58 | 400 | ip | 2.55 ± 0.37 | 28.2 |
| 59 | 60 | ip | 1.99 ± 0.70 | 23 |
| 60 | 60 | ip | 2.74 ± 0.27 | 25 |
| 61 | 100 | ip | 1.83 ± 0.58 | 34 |
| 62 | 10 | ip | 0.32 ± 0.24 | 49.71* |
| 64 | 8 | ip | 0.42 ± 0.24 | 42.92 |
| 65 | 45 | iv | 0.43 ± 0.24 | 41.61 |
| 66 | 120 | ip | 2.65 ± 0.59 | 36 |
| 67 | 50 | ip | 1.31 ± 0.31 | 51.67* |
| 68 | 70 | iv | 1.24 ± 0.73 | 54** |
| 69 | 200 | ip | 1.69 ± 0.63 | 28 |
| 70 | 10 | iv | 1.36 ± 0.75 | 40 |
| 71 | 200 | iv | 2.48 ± 0.37 | 11.47 |
| 73 | 25 | ip | 0.69 ± 0.60 | 33.28 |
| 74 | 100 | ip | 2.08 ± 0.63 | 20 |
| 75 | 200 | ip | 1.74 ± 0.59 | 30 |
| 76 | 30 | iv | 0.86 ± 0.48 | 34.35 |
| 78 | 60 | ip | 1.25 ± 0.33 | 38.68 |
| 87 | 120 | ip | 1.07 ± 0.71 | 35.15 |
| 88 | 20 | iv | 1.04 ± 0.70 | 38.79 |
| 91 | 80 | ip | 0.97 ± 0.51 | 41.12 |
| 93 | 80 | ip | 0.70 ± 0.32 | 35.19 |
| 94 | 15 | iv | 1.34 ± 0.48 | 25.56 |
| 97 | 15 | iv | 0.95 ± 0.25 | 32.02 |
| 98 | 100 | ip | 0.64 ± 0.19 | 40.74 |
| 100 | 30 | iv | 1.26 ± 0.56 | 38.11 |
| 105 | 8 | iv | 0.82 ± 0.32 | 40.25 |
| 107 | 60 | ip | 0.45 ± 0.19 | 49.33* |

*P < 0.01: compared with the control group significantly difference;
**p < 0.001: compared with the control group was very significant difference. Inhibition rate more than 40% of the sample was statistically significant better than control group.

1. A compound of the formula, 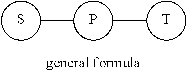
or stereoisomers, tautomers, prodrug, pharmaceutically acceptable salts, complex salts or solvates thereof, wherein:
S, P, T can be combined independently either in three or in two components to form SPT, STP, TSP, PT or ST structures, which is independently an optionally substituted with the carbon-carbon bond or carbon-hetero bond to form ethers, esters, amides, alcohols, aldehydes, ketones, amines, acetal, ketal, oxime and/or hydrazonyl;
where S is independently an optionally substituted cyclic group; P is an optionally substituted independently between S and T; T is independently an optionally substituted alkyl and/or adamantyl groups;
said S is independently and optionally substituted or fused, saturated or unsaturated, monocyclic, bicyclic, tricycli, teteracyclic, polycyclic, bridged cyclic group, a macrocyclic, midcyclic and/or small cyclic group to form C₃₋₃₀ arylcyclic, aliphatic cyclic, aliphatic heterocyclic group and/or aryl heterocyclic group as the structural formula I, II, III, IV, wherein: 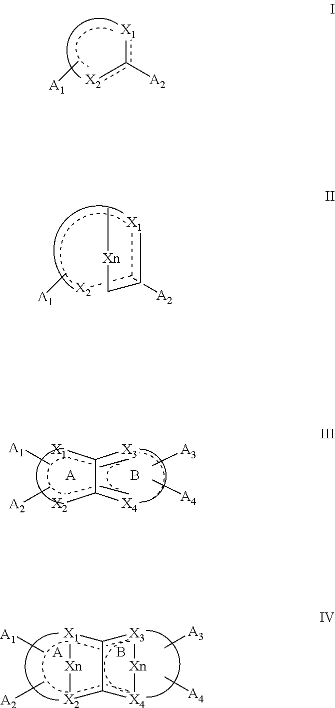
the ring A is optionally substituted independently C₃₋₁₈ alicyclic, arylcyclic, heterocyclic, aliphatic and/or heteroarylcyclic group; ring B is optionally substituted independently C₃₋₁₈ alicyclic, arylcyclic, heterocyclic, aliphatic and/or heteroarylcyclic group; licyclic, aryl-cyclic, and the aliphatic heterocyclic group or heteroarylcyclic group; A ring was fused with B ring directly or fused to form a bridged ring;
said P is an optionally substituted independently C₀₋₁₂ alkyl, C₀₋₁₈ aliphatic, C₀₋₁₈ cyclic, arylcyclic, aliphatic heterocyclic, aryl heterocyclic group between S and T to form an independent optionally substituted ether, ester, amide, alcohol, aldehyde, ketone, amine, acetal, ketal, oxime and/or hydrazone group by a formation of the carbon-carbon bond or a carbon-hetero bond with a certain interval of C₀₋₁₂ alkyl, C₀₋₁₈ linear or cyclic aliphatic, arylcyclic, aliphatic and arylheterocyclic, or a heterocyclic group between S and T;
said T is an optionally substituted independently adamantyl or adamantyl analog group which contains a C₃₋₃₀ monocyclic, bicyclic, polycycloalkyl, bridged cyclic, cage cyclic, fused cyclic or diamond group containing oxygen, sulfur, nitrogen, phosphine to form optionally substituted mono-adamantane, bi-adamantane, tri-adamantane, polyadamantane or adamantane caged analog with formula V, VI, VII, VIII, IX, X;

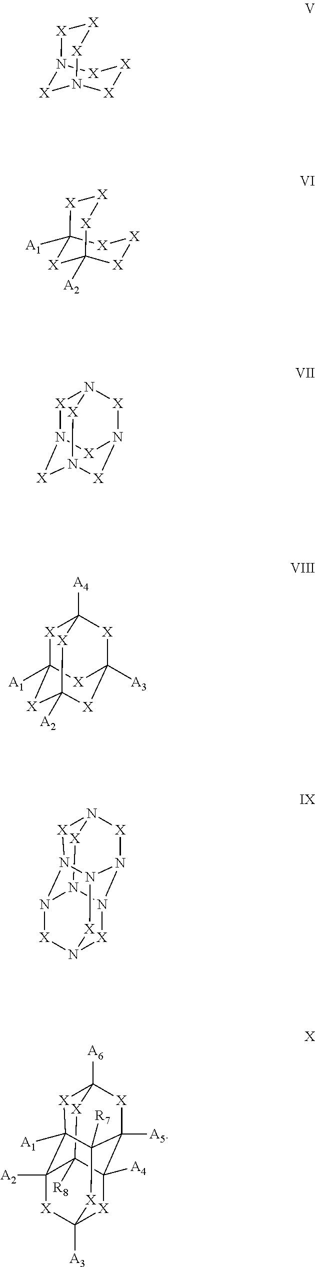

2. The compound according to the S formula of claim 1, wherein:
The dotted lines are optionally substituted single bonds, optionally substituted double bond or a optionally substituted heterocyclic group containing carbon, oxygen, sulfur or nitrogen element; said X₁, X₂, X₃, X₄ are, independently at each occurrence, C═O, C═S, C═NH, C═Rb—Ra, CHOH, CHORb, CHRb or substituent, where Rb contains, independently at each occurrence, one or combination of C, N or P element; Ra is H, H₂, optionally substituted straight-alkyl, optionally substituted branched-alkyl, C₁₋₁₀ optionally substituted saturated alkyl, optionally substituted 1-4 double bond, optionally substituted 1-4 triple bond, optionally substituted unsaturated alkyl, optionally substituted saturated or unsaturated alicyclic, optionally substituted arylcyclic, optionally substituted aryl or optionally substituted heterocyclic, optionally substituted arylheterocyclic, fused heterocyclic group where contains hydroxyl, halogen, oxygen, nitrogen, sulfur, phosphorus element or selenium element;
said A₁, A₂, A₃, A₄, A₅, A₆, A₇ or A₈ is, independently at each occurrence, optionally substituted independently cyclic, alkyl, aryl, alicyclic, heterocyclic, aliphatic, aromatic heterocyclic, heterocyclic, glycosyl, multi-hydroxyl, amino acid, phosphate, acyloxyl, phosphoric, sulfonyloxyl, alkoxy, aryloxyl, heterocyclic oxyl, aryl cyclic, aliphatic heterocyclic oxyl or aryl heterocyclic oxyl group, which containing hydrogen, halougen, oxygen, sulfur, nitrogen or phosphorus element, hydrogen bond, carbon-carbon bond, carbon-hetero bond, and one or a combination;
wherein said sugar or a glycoside bond with carbon-carbon and carbon-hetero atom linkage;
substituted oxygen-containing group, oxygen, sulfur, nitrogen or phosphorus, and substituents;
including optionally substituted 1-8 separate and independent sugar group or an optionally substituted glycosyl, the sugar group is independently an optionally substituted three-carbon sugar, tetroses, pentoses, hexoses, heptoses, monosaccharides, disaccharides, trisaccharides and polysaccharides or a group;
said substituted group is independently an optionally substituted acyloxy, 1-4 phosphono group, alkoxyl, aryloxyl or a heterocyclic; said substituent containing oxygen, sulfur, nitrogen or phosphorus atom, independently an optionally substituted unsaturated or saturated C₁₋₁₀ alkyl, 1-4 double bond or triple bond of the unsaturated aliphatic hydrocarbon group, saturated or unsaturated C₃₋₁₀ alkyl, aryl, alicyclic, heterocyclic, aryl heterocyclic, polycyclic group and or one of combination;
glycosyl, multi-hydroxyl, amino acid, phosphate, acyloxyl, phosphoric, sulfonyloxyl, alkoxy, aryloxyl, heterocyclic oxyl, aryl cyclic, aliphatic heterocyclic oxyl or aryl heterocyclic oxyl group, non-alicyclic group, an aromatic group or a heterocyclic group and, and the introduction of oxygen, sulfur, nitrogen or phosphorus atom independently 3-10 the carbon chain optionally substituted hydrocarbon group, an aromatic ring, polycyclic, aliphatic heterocyclic ring, fused aromatic heterocyclic ring or a heterocyclic cycle and one or a combination.

3. The compound according to the formation of S-P-T of claim 1, wherein:
is 7-(adamantan-1-ylamino)-5-(4-(trifluoromethyl)phenyl)pyrazolo[1,5-a]pyrimidine-3-carbonitrile, N-((3s,5s,7s)-adamantan-1-yl)-2-(5-nitro-1,3-dioxoisoindolin-2-yl)acetamide, N-((3s,5s,7s)-adamantan-1-yl)-2-(5-amino-1,3-dioxoisoindolin-2-yl)acetamide, N-((3s,5s,7s)adamantan-1-yl)-2-(1,3-dioxohexahydro-1H-4,7-epoxyisoindol-2(3H)-yl)propanamide, N-((1r,3r,5R,7S)-1-azaadamantan-3-yl)-2-(1,3-dioxohexahydro-1H-4,7-epoxyisoindol-2(3H)-yl)propanamide, N-((1s,3s)-1-azaadamantan-3-yl)-2-(1,3-dioxo-3a,4,7,7a-tetrahydro-1H-4,7-epoxyisoindol-2(3H)-yl)acetamide, N-((1r,3r)-1-azaadamantan-3-yl)-2-(5-hydroxy-1,3-dioxohexahydro-1H-4,7-epoxyisoindol-2(3H)-yl)acetamide, N-((1r,3r,5R,7S)-1-azaadamantan-3-yl)-2-(5-nitro-1,3-dioxoisoindolin-2-yl)acetamide, N-((1r,3r,5R,7S)-1-azaadamantan-3-yl)-2-(5-amino-1,3-dioxoisoindolin-2-yl)acetamide, (3S)—N-(adamantan-1-yl)-5-chloro-3-(2,4-dichlorophenyl)-2-methyl-6-phenylpyrazolo[1,5-a]pyrimidin-7-amine, N-((1s,3 s)-adamantan-1-yl)-2-(4,5-diphenyl-1H-imidazol-1-yl)acetamide, (1s,3s)-2-(2-(4,5-diphenyl-1H-imidazol-1-yl)acetamido)ethyl adamantane-1-carboxylate, (1s,3 s)-N-(4,5-diphenyl-1H-imidazol-2-yl)adamantane-1-carboxamide, N-((3s,5s,7s)-adamantan-1-yl)-3-(2,4-dichlorophenyl)-2,7-dimethylpyrazolo[1,5-a]pyrimidine-6-carboxamide, (3r,5r,7 r)-N-(6-cyano-3-(2,4-dichlorophenyl)-2-methylpyrazolo[1,5-a]pyrimidin-7-yl)adamantane-1-carboxamide, N-(2-((8-oxo-8H-phthalazino[1,2-b]quinazolin-5-yl)amino)ethyl)adamantane-1-carboxamide, N-((3s,5s,7s)adamantan-1-yl)-3-(2,4-dichlorophenyl)-2,6-dimethyl-5-(4-(trifluoromethyl)phenyl)pyrazolo[1,5-a]pyrimidin-7-amine, N-(adamantan-1-yl)-2-(2-(dimethylamino)ethoxy)acetamide, N-(adamantan-1-yl)-4-((3,4,5-trihydroxy-6-(hydroxymethyl)tetrahydro-2H-pyran-2-yl)oxy)benzamide, (3s,5s,7s)-N-(1-((2,2,2-trifluoro acetoxy)amino)propan-2-yl)adamantan-1-amine, N-(adamantan-1-yl)-2-(1,3-dioxohexahydro-1H-4,7-epoxyisoindol-2(3H)-yl)propanamide, N-((1S,3s)-adamantan-1-yl)-2-(5,6-dibromo-1,3-dioxohexahydro-1H-4,7-epoxyisoindol-2(3H)-yl)propanamide, N-(adamantan-1-yl)-2-(1,3-dioxoisoindolin-2-yl)propanamide, N-(adamantan-1-yl)-2-(5-nitro-1,3-dioxoisoindolin-2-yl)propanamide, N-(adamantan-1-yl)-2-(6-amino-1,3-dioxoisoindolin-2-yl)propanamide, (1s,3s,5s,7s)-adamantane-1,3-diol, (1s,3r,5R,7 S)-3-hydroxyadamantan-1-yl 2-chloro-5-nitrobenzoate, 1-azaadamantan-4-ol, 4-methylene-1-azaadamantane, 1-azaadamantane-4-carbonitrile, 1-azaadamantane-4-carboxylic acid, 1-azaadamantan-4-ylmethanamine, (1r,3s,5R,7S)-ethyl 1-azaadamantane-2-carboxylate, 1-azaadamantan-4-ylmethanol, 3,5,7-trinitro-1-azaadamantane, 1-azaadamantane-3,5,7-triamine, 3,5,7-tribromo-1-azaadamantane, 3,5,7-triallyl-1-azaadamantane-4,6,10-trione, 3,5,7-trimethyl-1-azaadamantane-4,6-dione, 1-boraadamantane, 1-aza-adamantane-1-oxide, 1-azaadamantane-4-thione, 2-oxaadamantan-4-one, (1S,3R,4S,5R,7S)-2-oxaadamantan-4-ol, 1-hydroxy-2-oxaadamantan-6-one, 6-amino-2-oxaadamantan-1-ol, 2-oxaadamantan-1-ol, (3s,5s,7s)-3,5,7-trimethyl-1-azaadamantane-4,6,10-trione, ((1S,3R,4R,5R,7 S)-4-hydroxy-2-azaadamantan-2-yl)(phenyl)methanone, (1S,3R,4R,5R,7S)-2-benzyl-2-azaadamantan-4-ol, (1s,3R,4R,5R,7 S)-2-azaadamantan-4-ol, (1R,3S)-2-benzo yl-2-azaadamantan-4-yl 4-methylbenzenesulfonate, (1R,3S)-2-benzoyl-2-azaadamantan-4-one, (1r,3R,5S,7s)-1-azaadamantan-4-one, (1R,3R,5S,7S,Z)-1-azaadamantan-4-one oxime, (1r,3R,5S,7s)-1-azaadamantan-4-amine, (1s,3s,5R,7S)-2-azaadamantan-1-ol, (1s,3s,5R,7S)-1-chloro-2-azaadamantane, (1r,3r,5r,7r)-2-azaadamantane, 2-((1r,3r,5r,7r)-2-azaadamantan-2-yl)ethanol, (1r,3r,5r,7r)-2-(2-chloroethyl)-2-azaadamantane, (1r,3r,5r,7r)-5,7-bis(3,4,5-trimethoxyphenyl)-1,3-diazaadamantan-6-one, (1r,3r,5r,7r)-5,7-bis(4-hydroxy-3-nitrophenyl)-1,3-diazaadamantan-6-one, (1r,3r,5r,7r)-5,7-bis(4-(3-hydroxy-4-nitrophenoxy)phenyl)-1,3-diazaadamantan-6-one, (1s,3s,5s,7s)-5,7-bis(3-hydroxy-4-nitrophenoxy)-1,3-diazaadamantan-6-one, (1s,3s,5s)-7-nitro-1,3,5-triazaadamantane, (1s,3s,5s)-1,3,5-triazaadamantan-7-amine, (1s,3s,5s)-N-pentyl-1,3,5-triazaadamantan-7-amine, N-((1s,3s,5s)-1,3,5-triazaadamantan-7-yl)-2-(5-amino-3-hydroxyhexahydro-4,7-epoxybenzo[d]isoxazol-2(3H)-yl)acetamide, (1s,3s,5s)-7-(piperidin-1-yl)-1,3,5-triazaadamantane, (1r,3s,5R,7S)-3-(hydroxymethyl)-2-oxaadamantan-1-ol, (1r,3s,5R,7S)-3-methyl-2-oxaspiro[adamantane-6,2′-[1,3]-dioxolan]-1-ol, (1r,3s,5R,7S)-1-hydroxy-3-methyl-2-oxaadamantan-6-one, (1R,3S,5R,7S,Z)-1-hydroxy-3-methyl-2-oxaadamantan-6-one oxime, (1S,3R,5R,7R)-5-amino-3-methyl-2-oxaadamantan-1-ol, (1R,3S,5s,7s)-2-oxaadamantan-5-ol, (1r,3r,5s,7s)-2-oxaadamantane-5,7-diyl diacetate, (1r,3r,5s,7s)-2-oxaadamantane-5,7-diol, (1s,3s,5R,7S)-1-bromo-2-oxaadamantane, (1r,3s,5R,7S)—N-benzyl-2-oxaadamantan-1-amine, (1r,3s,5R,7S)—N-phenethyl-2-oxaadamantan-1-amine, (1r,3s,5R,7S)—N-benzyl-N-methyl-2-oxaadamantan-1-amine, (1r,3s,5R,7S)—N-methyl-N-phenethyl-2-oxaadamantan-1-amine, (1r,3s,5R,7S)—N-methyl-2-oxaadamantan-1-amine, (1r,3s,5R,7S)-2-oxaadamantan-1-amine, (1r,3s,5R,7S)—N,N-dibenzyl-2-oxaadamantan-1-amine, ((1r,3s,5R,7S)-3-methyl-2-oxaadamantan-1-yl)hydrazine, (1r,3s,5R,7S)-3-methyl-2-oxaadamantan-1-amine, (1r,3s,5R,7S)—N,N,3-trimethyl-2-oxaadamantan-1-amine, (1r,3s,5R,7S)—N,N,3-triethyl-2-oxaadamantan-1-amine, (1r,3s,5R,7S)—N-benzyl-3-ethyl-2-oxaadamantan-1-amine, (1r,3s,5R,7S)—N-benzyl-3-ethyl-N-methyl-2-oxaadamantan-1-amine, (1r,3s,5R,7S)-3-ethyl-N-methyl-2-oxaadamantan-1-amine, (1R,3s,5S,7r)-2,4-dioxaadamantane, (1R,3R,5S,6R,7S,8R,9S)-9-hydroxy-2,4,10-trioxaadamantane-6,8-diyl dibenzoate, (1S,4S)-(1S,3R,5R,6S,7S,8R,9R)-8,9-bis(tosyloxy)-2,4,10-trioxaadamantan-6-yl 4,7,7-trimethyl-3-oxo-2-oxabicyclo[2.2.1]heptane-1-carboxylate, (1S,4S)-(1R,3S,5R,6R,7S,8S,9S)-8,9-bis(tosyloxy)-2,4,10-trioxaadamantan-6-yl 4,7,7-trimethyl-3-oxo-2-oxabicyclo[2.2.1]heptane-1-carboxylate, 3,5-dihydroxy-4-((3-hydroxy-5-(hydroxymethyl)-2-methylpyridin-4-yl)methylene)cyclohexa-2,5-dienone, 3-(3-hydroxy-5-(hydroxymethyl)-2-methylpyridin-4-yl)-2,4,10-trioxaadamantane-1,5,7-triol, 7-benzoyl-3-tosyl-3-azabicyclo[3.3.1]nonan-9-one, 7-(hydroxy(phenyl)methyl)-3-tosyl-3-azabicyclo[3.3.1]nonan-9-ol, (1r,3R,5S,7s)-8-methyl-8-phenyl-1-azaadamantan-4-one, (1R,5S,7s)-ethyl 9-oxo-3-tosyl-3-azabicyclo[3.3.1]nonane-7-carboxylate, (1R,5S,7s)-ethyl 3-tosyl-3-azaspiro[bicyclo[3.3.1]nonane-9,2′-[1,3]dithiolane]-7-carboxylate, (1R,5S,7s)-ethyl 3-tosyl-3-azabicyclo[3.3.1]nonane-7-carboxylate, ((1R,5S,7s)-3-tosyl-3-azabicyclo[3.3.1]nonan-7-yl)methanol, 1,3,5,7-tetramethyl-2,4,6,8,9,10-hexathiaadamantane, tetramethanol decahydro-2,8,4,6-(cyclobutyl[1,2,3,4]dimethyl)naphthalene-2,4,4a,6-tetracarboxylate, dimethanol decahydro-2,8,4,6-(cyclobutyl[1,2,3,4]dimethyl)naphthalene-2,4,4a,6-dicarboxylate, N-((1r,3r,5R,7S)-1-azaadamantan-3-yl)-2-(5-hydroxy-1,3-dioxohexahydro-1H-4,7-epoxyisoindol-2(3H)-yl)acetamide, N-((1r,3r,5R,7S)-1-azaadamantan-3-yl)-2-(5-amino-1,3-dioxoisoindolin-2-yl)acetamide, 7-((3s,5s,7s)-adamantan-1-ylamino)-2-methyl-5-(4-(trifluoromethyl)phenyl)pyrazolo[1,5-a]pyridine-3-carbonitrile, (1s,3 s)-2-(2-(4,5-diphenyl-1H-imidazol-1-yl)acetamido)ethyl adamantane-1-carboxylate, N-(adamantan-1-yl)-2-(1,3-dioxohexahydro-1H-4,7-epoxyisoindol-2(3H)-yl)propanamide, N-(adamantan-1-yl)-2-(5,6-dibromo-1,3-dioxohexahydro-1H-4,7-epoxyisoindol-2(3H)-yl)propanamide, N-(adamantan-1-yl)-2-(1,3-dioxoisoindolin-2-yl)propanamide, N-(adamantan-1-yl)-2-(6-nitro-1,3-dioxoisoindolin-2-yl)propanamide, 3,5,7-trimethyl-1-azaadamantane-4,6-diol, (1s,3R,5r,7S)-3,5,7-trimethyl-1-azaadamantane-4,6-dione, (1S,3R,4R,5R,6R,7S)-3,5,7-trimethyl-1-azaadamantane-4,6-diol, (3s,5s,7s)-1-azaadamantane, 2-oxaadamantan-4-ol, bicyclo[3.3.1]non-6-en-3-ol, (1s,5s,7s)-3-(3-hydroxy-5-(hydroxymethyl)-2-methylpyridin-4-yl)-2,4,10-trioxaadamantane-1,5,7-triol, N-((3s,5s,7s)-adamantan-1-yl)-2-(N-formylformamido)acetamide, N-((3s,5s,7s)-adamantan-1-yl)-2-(2-hydroxy-5-oxo-2,5-dihydro-1H-pyrrol-1-yl)acetamide, N-((3s,5s,7s)-adamantan-1-yl)-2-(2-oxo-2,5-dihydro-1H-pyrrol-1-yl)acetamide, N-((3s,5s,7s)-adamantan-1-yl)-2-(2,5-dioxopyrrolidin-1-yl)acetamide, N-((3s,5s,7s)-adamantan-1-yl)-2-(2-hydroxy-5-oxopyrrolidin-1-yl)acetamide, N-((3s,5s,7s)-adamantan-1-yl)-2-(2-oxopyrrolidin-1-yl)acetamide, N-((3s,5s,7s)-adamantan-1-yl)-2-(5-amino-3-hydroxy-1-oxoisoindolin-2-yl)acetamide, N-((3s,5s,7s)-adamantan-1-yl)-2-(5-amino-1-oxoisoindolin-2-yl)acetamide, N-((3s,5s,7s)-adamantan-1-yl)-2-(5-amino-1,3-dioxohexahydro-1H-4,7-epoxyisoindol-2(3H)-yl)acetamide, N-((3s,5s,7s)-adamantan-1-yl)-2-(5-amino-3-hydroxy-1-oxohexahydro-1H-4,7-epoxyisoindol-2(3H)-yl)acetamide, N-((3s,5s,7s)-adamantan-1-yl)-2-(5-amino-1-oxohexahydro-1H-4,7-epoxyisoindol-2(3H)-yl)acetamide, ((1S,3R,4R,5R,7S)-4-hydroxy-2-azaadamantan-2-yl)(phenyl)methanone, (1r,3R,5S,7s)-1-azaadamantan-4-one, (1r,3R,5S,7s)-1-azaadamantan-4-amine, 2-((1r,3r,5r,7r)-2-azaadamantan-2-yl)ethanol, (1s,3s,5R,7S)-2-azaadamantan-1-ol, (1s,3s,5R,7S)-1-chloro-2-azaadamantane, 2-((1r,3r,5r,7r)-2-azaadamantan-2-yl)ethanol, (1r,3r,5r,7r)-2-(2-chloroethyl)-2-azaadamantane, N-((1r,3r,5R,7S)-1-azaadamantan-3-yl)-2-(N-formylformamido)acetamide, N-((1r,3r,5R,7S)-1-azaadamantan-3-yl)-2-(2-hydroxy-5-oxo-2,5-dihydro-1H-pyrrol-1-yl)acetamide, N-((1r,3r,5R,7S)-1-azaadamantan-3-yl)-2-(2-oxo-2,5-dihydro-1H-pyrrol-1-yl)acetamide, N-((1r,3r,5R,7S)-1-azaadamantan-3-yl)-2-(2,5-dioxopyrrolidin-1-yl)acetamide, N-((1r,3r,5R,7S)-1-azaadamantan-3-yl)-2-(2-hydroxy-5-oxopyrrolidin-1-yl)acetamide, N-((1r,3r,5R,7S)-1-azaadamantan-3-yl)-2-(2-oxopyrrolidin-1-yl)acetamide, N-((1s,3s,5R,7S)-2-azaadamantan-1-yl)-2-(2,5-dioxo-2,5-dihydro-1H-pyrrol-1-yl)acetamide, N-((1s,3s,5R,7S)-2-azaadamantan-1-yl)-2-(2-hydroxy-5-oxo-2,5-dihydro-1H-pyrrol-1-yl)acetamide, N-((1s,3s,5R,7S)-2-azaadamantan-1-yl)-2-(2-oxo-2,5-dihydro-1H-pyrrol-1-yl)acetamide, N-((1s,3s,5R,7S)-2-azaadamantan-1-yl)-2-(2,5-dioxopyrrolidin-1-yl)acetamide, N-((1s,3s,5R,7S)-2-azaadamantan-1-yl)-2-(2-hydroxy-5-oxopyrrolidin-1-yl)acetamide, N-((1s,3s,5R,7S)-2-azaadamantan-1-yl)-2-(2-oxopyrrolidin-1-yl)acetamide, N-((1r,3r,5R,7S)-1-azaadamantan-3-yl)-2-(5-amino-1,3-dioxoisoindolin-2-yl)acetamide, N-((1r,3r,5R,7S)-1-azaadamantan-3-yl)-2-(5-amino-3-hydroxy-1-oxoisoindolin-2-yl)acetamide, N-((1r,3r,5R,7S)-1-azaadamantan-3-yl)-2-(5-amino-1-oxoisoindolin-2-yl)acetamide, N-((1r,3r,5R,7S)-1-azaadamantan-3-yl)-2-(5-amino-1,3-dioxohexahydro-1H-4,7-epoxyisoindol-2(3H)-yl)acetamide, N-((1r,3r,5R,7S)-1-azaadamantan-3-yl)-2-(5-amino-3-hydroxy-1-oxohexahydro-1H-4,7-epoxyisoindol-2(3H)-yl)acetamide, N-((1r,3r,5R,7S)-1-azaadamantan-3-yl)-2-(5-amino-1-oxohexahydro-1H-4,7-epoxyisoindol-2(3H)-yl)acetamide, N-((1s,3s,5R,7S)-2-azaadamantan-1-yl)-2-(5-amino-1,3-dioxoisoindolin-2-yl)acetamide, N-((1s,3s,5R,7S)-2-azaadamantan-1-yl)-2-(5-amino-3-hydroxy-1-oxoisoindolin-2-yl)acetamide, N-((1s,3s,5R,7S)-2-azaadamantan-1-yl)-2-(5-amino-1-oxoisoindolin-2-yl)acetamide, N-((1s,3s,5R,7S)-2-azaadamantan-1-yl)-2-(5-amino-1,3-dioxohexahydro-1H-4,7-epoxyisoindol-2(3H)-yl)acetamide, N-((1s,3s,5R,7S)-2-azaadamantan-1-yl)-2-(5-amino-3-hydroxy-1-oxohexahydro-1H-4,7-epoxyisoindol-2(3H)-yl)acetamide, N-((1s,3s,5R,7S)-2-azaadamantan-1-yl)-2-(5-amino-1-oxohexahydro-1H-4,7-epoxyisoindol-2(3H)-yl)acetamide, 5,5′-((1r,3R,5S,7s)-1-azaadamantane-4,4-diylbis(oxy))bis(2-nitrophenol), 2-hydroxy-4-(((1r,3R,4r,5S,7s)-4-(3-hydroxy-4-nitrophenoxy)-1-azaadamantan-4-yl)oxy)benzonitrile, 5,5′-(((1s,3R,5S,7r)-1-azaadamantane-4,4-diylbis(4,1-phenylene))bis(oxy))bis(2-nitrophenol), 2-hydroxy-4-(4-((1s,3R,4s,5S,7r)-4-(4-(3-hydroxy-4-nitrophenoxy)phenyl)-1-azaadamantan-4-yl)phenoxy)benzonitrile, 5,5′-((1r,3R,5S,7s)-1-azaadamantane-3,5-diylbis(oxy))bis(2-nitrophenol), 2-hydroxy-4-(((1S,3R,5S,7R)-5-(3-hydroxy-4-nitrophenoxy)-1-azaadamantan-3-yl)oxy)benzonitrile, 5,5′-(((1s,3R,5S,7r)-1-azaadamantane-3,5-diylbis(4,1-phenylene))bis(oxy))bis(2-nitrophenol), 5,5′-((1r,3r,5r,7r)-1,3-diazaadamantane-6,6-diylbis(oxy))bis(2-nitrophenol), 2-hydroxy-4-(((1r,3r,5R,7S)-6-(3-hydroxy-4-nitrophenoxy)-1,3-diazaadamantan-6-yl)oxy)benzonitrile, 5,5′-(((1r,3r,5r,7r)-1,3-diazaadamantane-6,6-diylbis(4,1-phenylene))bis(oxy))bis(2-nitrophenol), 2-hydroxy-4-(4-(1r,3r,5R,7S)-6-(4-(3-hydroxy-4-nitrophenoxy)phenyl)-1,3-diazaadamantan-6-yl)phenoxy)benzonitrile, 5,5′-((1s,3s,5s,7s)-1,3-diazaadamantane-5,7-diylbis(oxy))bis(2-nitrophenol), 2-hydroxy-4-(((1R,3S,5r,7s)-7-(3-hydroxy-4-nitrophenoxy)-1,3-diazaadamantan-5-yl)oxy)benzonitrile, 5,5′-(((1s,3s,5s,7s)-1,3-diazaadamantane-5,7-diylbis(4,1-phenylene))bis(oxy))bis(2-nitrophenol), 2-hydroxy-4-(4-((1R,3S,5r,7s)-7-(4-(3-hydroxy-4-nitrophenoxy)phenyl)-1,3-diazaadamantan-5-yl)phenoxy)benzonitrile, 5,5′-((1R,3S,5s,7r)-1,3,5-triazaadamantane-6,6-diylbis(oxy))bis(2-nitrophenol), 2-hydroxy-4-(((1R,3S,5S,6R,7R)-6-(3-hydroxy-4-nitrophenoxy)-1,3,5-triazaadamantan-6-yl)oxy)benzonitrile, 5,5′-(((1R,3S,5s,7r)-1,3,5-triazaadamantane-6,6-diylbis(4,1-phenylene))bis(oxy))bis(2-nitrophenol), 2-hydroxy-4-(4-((1R,3S,5S,6R,7R)-6-(4-(3-hydroxy-4-nitrophenoxy)phenyl)-1,3,5-triazaadamantan-6-yl)phenoxy)benzonitrile, N-((1s,3s,5s)-1,3,5-triazaadamantan-7-yl)-2-(5-amino-1,3-dioxoisoindolin-2-yl)acetamide, N-((1s,3s,5s)-1,3,5-triazaadamantan-7-yl)-2-(5-amino-3-hydroxy-1-oxoisoindolin-2-yl)acetamide, N-((1s,3s,5s)-1,3,5-triazaadamantan-7-yl)-2-(5-amino-1-oxoisoindolin-2-yl)acetamide, N-((1s,3s,5s)-1,3,5-triazaadamantan-7-yl)-2-(5-amino-1,3-dioxohexahydro-1H-4,7-epoxyisoindol-2(3H)-yl)acetamide, N-((1s,3s,5s)-1,3,5-triazaadamantan-7-yl)-2-(5-amino-3-hydroxy-1-oxohexahydro-1H-4,7-epoxyisoindol-2(3H)-yl)acetamide, N-((1s,3s,5s)-1,3,5-triazaadamantan-7-yl)-2-(5-amino-1-oxohexahydro-1H-4,7-epoxyisoindol-2(3H)-yl)acetamide, N-((1s,3s,5s)-1,3,5-triazaadamantan-7-yl)-2-(2,5-dioxo-2,5-dihydro-1H-pyrrol-1-yl)acetamide, N-((1s,3s,5s)-1,3,5-triazaadamantan-7-yl)-2-(2-hydroxy-5-oxo-2,5-dihydro-1H-pyrrol-1-yl)acetamide, N-((1s,3s,5s)-1,3,5-triazaadamantan-7-yl)-2-(2-oxo-2,5-dihydro-1H-pyrrol-1-yl)acetamide, N-((1s,3s,5s)-1,3,5-triazaadamantan-7-yl)-2-(2,5-dioxopyrrolidin-1-yl)acetamide, N-((1s,3s,5s)-1,3,5-triazaadamantan-7-yl)-2-(2-hydroxy-5-oxopyrrolidin-1-yl)acetamide, N-((1s,3s,5s)-1,3,5-triazaadamantan-7-yl)-2-(2-oxopyrrolidin-1-yl)acetamide, (1r,3s,5R,7S)-1-hydroxy-2-oxaadamantan-6-one, (1r,3s,5R,7S)-3-(hydroxymethyl)-2-oxaadamantan-1-ol, (1S,3R,5R,7R)-5-amino-2-oxaadamantan-1-ol, (1r,3r,5s,7s)-2-oxaadamantane-5,7-diol, (3s,5s,7s)-3,5,7-trimethyl-1-azaadamantane-4,6,10-trione, (1r,3s,5R,7S)—N,N-dibenzyl-2-oxaadamantan-1-amine, (1r,3s,5R,7S)-1-hydroxy-3-methyl-2-oxaadamantan-6-one, (1r,3s,5R,7S)-3-(hydroxymethyl)-2-oxaadamantan-1-ol, (1r,3r,5s,7s)-2-oxaadamantane-5,7-diol, (1s,3s,5R,7S)-1-bromo-2-oxaadamantane, (1R,3S,5s,7s)-2-oxaadamantan-5-ol, 5,5′-(((1s,3s,5s,7s)-2-oxaadamantane-5,7-diylbis(4,1-phenylene))bis(oxy))bis(2-nitrophenol), 2-hydroxy-4-(4-((1R,3S,5r,7s)-7-(4-(3-hydroxy-4-nitrophenoxy)phenyl)-2-oxaadamantan-5-yl)phenoxy)benzonitrile, 5,5′-((1r,3r,5s,7s)-2-oxaadamantane-5,7-diylbis(oxy))bis(2-nitrophenol), 2-hydroxy-4-(((1R,3S,5r,7s)-7-(3-hydroxy-4-nitrophenoxy)-2-oxaadamantan-5-yl)oxy)benzonitrile, N-((1r,3s,5R,7S)-2-oxaadamantan-1-yl)-2-(5-amino-1,3-dioxoisoindolin-2-yl)acetamide, N-((1r,3s,5R,7S)-2-oxaadamantan-1-yl)-2-(5-amino-3-hydroxy-1-oxoisoindolin-2-yl)acetamide, N-((1r,3s,5R,7S)-2-oxaadamantan-1-yl)-2-(5-amino-1-oxoisoindolin-2-yl)acetamide, N-((1r,3s,5R,7S)-2-oxaadamantan-1-yl)-2-(5-amino-1,3-dioxohexahydro-1H-4,7-epoxyisoindol-2(3H)-yl)acetamide, N-((1r,3s,5R,7S)-2-oxaadamantan-1-yl)-2-(5-amino-3-hydroxy-1-oxohexahydro-1H-4,7-epoxyisoindol-2(3H)-yl)acetamide, N-((1r,3s,5R,7S)-2-oxaadamantan-1-yl)-2-(5-amino-1-oxohexahydro-1H-4,7-epoxyisoindol-2(3H)-yl)acetamide, N-((1r,3s,5R,7S)-2-oxaadamantan-1-yl)-2-(2,5-dioxo-2,5-dihydro-1H-pyrrol-1-yl)acetamide, N-((1r,3s,5R,7S)-2-oxaadamantan-1-yl)-2-(2-hydroxy-5-oxo-2,5-dihydro-1H-pyrrol-1-yl)acetamide, N-((1r,3s,5R,7S)-2-oxaadamantan-1-yl)-2-(2-oxo-2,5-dihydro-1H-pyrrol-1-yl)acetamide, N-((1r,3s,5R,7S)-2-oxaadamantan-1-yl)-2-(2,5-dioxopyrrolidin-1-yl)acetamide, N-((1r,3s,5R,7S)-2-oxaadamantan-1-yl)-2-(2-hydroxy-5-oxopyrrolidin-1-yl)acetamide, N-((1r,3s,5R,7S)-2-oxaadamantan-1-yl)-2-(2-oxopyrrolidin-1-yl)acetamide, 5,5′-(((1R,3S,5S,7S)-2,4-dioxaadamantane-1,7-diylbis(4,1-phenylene))bis(oxy))bis(2-nitrophenol), 2-hydroxy-4-(4-((1R,3S,5S,7S)-1-(4-(3-hydroxy-4-nitrophenoxy)phenyl)-2,4-dioxaadamantan-7-yl)phenoxy)benzonitrile, 5,5′-((1S,3S,5S,7R)-2,4-dioxaadamantane-1,7-diylbis(oxy))bis(2-nitrophenol), 2-hydroxy-4-(((1S,3S,5S,7R)-1-(3-hydroxy-4-nitrophenoxy)-2,4-dioxaadamantan-7-yl)oxy)benzonitrile, N-((1R,3r,5S,7r)-2,4-dioxaadamantan-3-yl)-2-(2,5-dioxo-2,5-dihydro-1H-pyrrol-1-yl)acetamide, N-((1R,3r,5S,7r)-2,4-dioxaadamantan-3-yl)-2-(2-hydroxy-5-oxo-2,5-dihydro-1H-pyrrol-1-yl)acetamide, N-((1R,3r,5S,7r)-2,4-dioxaadamantan-3-yl)-2-(2-oxo-2,5-dihydro-1H-pyrrol-1-yl)acetamide, N-((1R,3r,5S,7r)-2,4-dioxaadamantan-3-yl)-2-(2,5-dioxopyrrolidin-1-yl)acetamide, N-((1R,3r,5S,7r)-2,4-dioxaadamantan-3-yl)-2-(2-hydroxy-5-oxopyrrolidin-1-yl)acetamide, N-((1R,3r,5S,7r)-2,4-dioxaadamantan-3-yl)-2-(2-oxopyrrolidin-1-yl)acetamide, 5,5′-(((1R,3s,5S,7r)-2,4,10-trioxaadamantane-1,5-diylbis(4,1-phenylene))bis(oxy))bis(2-nitrophenol), 2-hydroxy-4-(4-((1R,3R,5S,7S)-5-(4-(3-hydroxy-4-nitrophenoxy)phenyl)-2,4,10-trioxaadamantan-1-yl)phenoxy)benzonitrile, 5,5′-((1R,3s,5S,7r)-2,4,10-trioxaadamantane-1,5-diylbis(oxy))bis(2-nitrophenol), 2-hydroxy-4-(((1R,3R,5S,7S)-5-(3-hydroxy-4-nitrophenoxy)-2,4,10-trioxaadamantan-1-yl)oxy)benzonitrile, N-((1s,5s,7s)-2,4,10-trioxaadamantan-3-yl)-2-(5-amino-1,3-dioxoisoindolin-2-yl)acetamide, N-((1s,5s,7s)-2,4,10-trioxaadamantan-3-yl)-2-(5-amino-3-hydroxy-1-oxoisoindolin-2-yl)acetamide, N-((1s,5s,7s)-2,4,10-trioxaadamantan-3-yl)-2-(5-amino-1-oxoisoindolin-2-yl)acetamide, N-((1s,5s,7s)-2,4,10-trioxaadamantan-3-yl)-2-(5-amino-1,3-dioxohexahydro-1H-4,7-epoxyisoindol-2(3H)-yl)acetamide, N-((1s,5s,7s)-2,4,10-trioxaadamantan-3-yl)-2-(5-amino-3-hydroxy-1-oxohexahydro-1H-4,7-epoxyisoindol-2(3H)-yl)acetamide, N-((1s,5s,7s)-2,4,10-trioxaadamantan-3-yl)-2-(5-amino-1-oxohexahydro-1H-4,7-epoxyisoindol-2(3H)-yl)acetamide, N-((1s,5s,7s)-2,4,10-trioxaadamantan-3-yl)-2-(2,5-dioxo-2,5-dihydro-1H-pyrrol-1-yl)acetamide, N-((1s,5s,7s)-2,4,10-trioxaadamantan-3-yl)-2-(2-hydroxy-5-oxo-2,5-dihydro-1H-pyrrol-1-yl)acetamide, N-((1s,5s,7s)-2,4,10-trioxaadamantan-3-yl)-2-(2-oxo-2,5-dihydro-1H-pyrrol-1-yl)acetamide, N-((1s,5s,7s)-2,4,10-trioxaadamantan-3-yl)-2-(2,5-dioxopyrrolidin-1-yl)acetamide, N-((1s,5s,7s)-2,4,10-trioxaadamantan-3-yl)-2-(2-hydroxy-5-oxopyrrolidin-1-yl)acetamide, N-((1s,5s,7s)-2,4,10-trioxaadamantan-3-yl)-2-(2-oxopyrrolidin-1-yl)acetamide.

4. The compound according to claim 1, wherein:
The compound is selected from the exemplified examples or stereoisomers, tautomers, pharmaceutically acceptable salts, inorganic acid salt, organic acid salt, organic basic salt, organic basic salt, complex salt, prodrug or solvates thereof in association with a pharmaceutically acceptable excipient or carrier.

5. A method according to the claim 1, wherein:
the compound for treating, preventing or slowing the progression of neoplasia and cancer, and infection diseases comprises the examples, isomers, stereoisomers, prodrugs, pharmaceutically acceptable salts, complex salts, solvates of the compounds or pharmaceutical formulations and carriers.

6. A method according to the claim 1, wherein:
The compound for treating, preventing or slowing the progression of neoplasia and cancer, and infection diseases by virus, bacterial or fungi, including bacterial infections and fungal infections of the drug application, which comprises administration together with at least one known chemotherapeutic agent selected from the group consisting of antibacterial and antifungal drugs to a patient in need of such treatment.

7. A method according to the claim 1, wherein:
the method for treating cancer, comprising: administration to a compound of the claim 1, in the range of 0.001 mg/kg-250 mg/kg, a pharmaceutically acceptable salt or prodrug from thereof; a cancer is selected from the lung cancer, stomach cancer, colon cancer, small cell lung cancer, thyroid cancer, esophageal cancer, pancreatic cancer, endometrial cancer, adrenal cortical carcinoma, head and neck cancer, Osteogenic sarcoma, breast cancer, ovarian cancer, Vail Williams tumors, cervical tumors, testicular cancer, genitourinary cancer, skin cancer, renal cell carcinoma, bladder cancer, primary brain cancer, prostate cancer, soft tissue sarcoma, neuroblastoma, rhabdomyosarcoma, Kaposi sarcoma, malignant melanoma, malignant pancreatic islet tumors, nonHodgkin's lymphoma, malignant melanoma, multiple myeloma, neuroblastoma, malignant carcinoid cancer, choriocarcinoma, acute and chronic lymphocytic leukemia, primary macroglobulinemia, chronic myeloid leukemia, chronic lymphocytic leukemia, acute myeloid leukemia, hairy cell leukemia, mycosis fungoides, malignant hypercalcemia, cervical hyperplasia, or Hodgkin's disease.

8. The method according to claim 7, wherein:
said compounds is administered together with at least one known cancer, chemotherapeutic and immune agent selected from cyclophosphamide, vincristine, busulfan, vinblastine, cisplatin, carboplatin, mitomycin C, doxorubicin, colchicine, etoposide, paclitaxel, docetaxel, camptothecin, topotecan, arsenic trioxide, 5-azacytidine, 5-fluorouracil, methotrexate, 5-fluoro-2-deoxyuridine, hydroxyurea, thioguanine, melphalan, chlorambucil, ifosfamide, mitoguazone, epirubicin, aclarubicin, bleomycin, mitoxantrone, elliptinium acetate, fludarabine, octreotide, retinoic acid, tamoxifen, doxazosin, terazosin tamsulosin, tamsulosin, fluorine pyridinoline, lovastatin, simvastatin, pravastatin, fluvastatin, atorvastatin, atorvastatin, amprenavir, abacavir, flavonoids pyridinoline, ritonavir, saquinavir, rofecoxib, alanosine, retinal, tretinoin tocoferil, 13-cis-retinoic acid, 9-cis-retinoic acid, α-difluoro-methyl ornithine, fenretinide, N-4-carboxyphenyl retinamide, genistein, ara-C, CB-64D, CB-184, ILX23-7553, lactacystin, MG-132, PS-341, Glcevec, ZD1839 (IRessa), SH268, Herceptin, Rituxan, Gamcitabine, ABT-378, AG1776, BMS-232, 632, CEP2563, SU6668, EMD121974, R115777, SCH66336, L-778, 123, BAL9611, TAN-1813, UCN-01, Roscovitine, Olonoucine, Valecoxib.

9. The compound according to the claim 8, wherein:
the administration may be by oral route, parenteral, subcutaneous, intravenous, intramuscular, intra-peritoneal, transdermal, buccal, intrathecal, intracranial, intranasal or topical routes.
